SEQUENCE LISTING (1 sequence)

SEQ ID NO: 1 - protein, 9 aa, synthetic construct
SVYDFFVWL

(19) United States
(12) Patent Application Publication
(10) Pub. No.: US 2026/0224680 A1
(43) Pub. Date: Aug. 6, 2026

(54) COMPOSITIONS FOR INDUCING OR RESCUING GLYCOLYSIS AND METHODS OF USE THEREOF

(71) Applicant: Arizona Board of Regents on behalf of Arizona State University, Scottsdale, AZ (US)

(72) Inventor: Abhinav Acharya, Tempe, AZ (US)

(21) Appl. No.: 19/147,102

(22) PCT Filed: Jan. 10, 2024
(86) PCT No.: PCT/US24/11014

§ 371 Date: Jul. 10, 2025

Related U.S. Application Data
(60) Provisional application No. 63/479,280, filed on Jan. 10, 2023.

(51) Int. Cl. A61K 39/00; A61K 9/50; A61K 31/4709; A61K 40/11; A61K 45/06; A61K 47/54; A61P 35/00

(52) U.S. Cl. CPC A61K 39/001156; A61K 9/5015; A61K 31/4709; A61K 40/11; A61K 45/06; A61K 47/549; A61P 35/00; A61K 2039/53; A61K 2039/55555; A61K 2039/55561; A61K 2039/572

(57) ABSTRACT

The present invention provides polymers, particles (e.g., microparticles or particles), and compositions thereof for inducing an immune response, preventing or treating a metabolic inhibition in a subject, preventing or treating a disease or disorder, such as cancer, and/or adoptive T cell therapy. The present invention additionally provides kits that find use in the practice of the methods of the invention.

### CROSS-REFERENCE TO RELATED APPLICATIONS

This application claims priority to and the benefit of U.S. Provisional Application No. 63/479,280 filed Jan. 10, 2023, the disclosure of which is incorporated herein by reference in its entirety.

### STATEMENT OF GOVERNMENT FUNDING

This invention was made with government support under NIH R01AI155907 awarded by the National Institutes of Health. The government has certain rights in the invention.

### REFERENCE TO A “SEQUENCE LISTING” SUBMITTED AS AN XML FILE

The present application hereby incorporates by reference the entire contents of the XML file named “206339-0085-00WO_SequenceListing.xml” which was created on Jan. 10, 2024 and is 2,038 bytes in size.

### BACKGROUND OF THE INVENTION

Glycolysis inhibition in cancer cells has been long recognized as a viable strategy to prevent cancer cell growth. Cancer cells are known to have a high glucose consumption rate as a metabolism adaptation mechanism called Warburg effect (or aerobic glycolysis), where these cells generate adenosine triphosphate (ATP) from glycolysis rather than oxidative phosphorylation (OXPHOS) even in the presence of high oxygen supply. Thus, several pharmacological approaches have been tested to prevent glycolysis in cancer cells that include drugs, such as trametinib, vemurafenib, and dacarbazine as well as targeting various glycolytic enzymes and transporters. Activated immune cells also rely on glycolysis for their energy needs. For example, glycolysis is essential for dendritic cells (DCs) to get activated and generate tumor necrosis factor alpha (TNFα), which helps prime T cells against melanoma tumors for recognition and their eventual elimination. However, blocking glycolysis in cancer cells while simultaneously allowing glycolysis to occur in DCs is a major challenge in immunometabolism and cancer immunotherapy.

Thus, there is a need in the art for novel compositions, which selectively block glycolysis in cancer cells and simultaneously enable glycolysis in non-cancer cells (e.g., DCs, macrophages, T cells, B cells, natural killer cells, neutrophils, eosinophils, basophils, or any other immune cells) as well as methods of use thereof. The present invention satisfies this unmet need.

### BRIEF SUMMARY OF THE INVENTION

In one aspect, the present invention relates, in part, to a composition comprising a particle, wherein the particle comprises a) a nucleic acid molecule or a fragment or variant thereof comprising a nucleotide sequence that encodes at least one antigen or a fragment or variant thereof, and b) a compound comprising the structure of Formula (I)

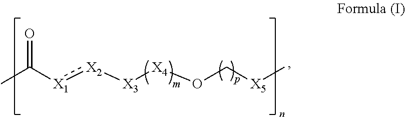

or a derivative or a pharmaceutical acceptable salt thereof.

In some embodiments, the compound comprising the structure of Formula (I) is a compound comprising the structure of Formula (II)

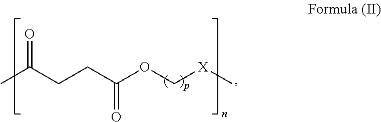

or a derivative or a pharmaceutical acceptable salt thereof.

In some embodiments, each occurrence of X is independently O, S, C═R₁, CR₃R₄, NR₂, PR₂, or P(═R₁)(R₂).

In some embodiments, each occurrence of X₁ and X₂ is independently C═R₁, CR₂, or CR₃R₄.

In some embodiments, each occurrence of X₃ and X₄ is independently C═R₁ or CR₃R₄.

In some embodiments, each occurrence of X₅ is independently O, S, C═R₁, CR₃R₄, NR₂, PR₂, or P(═R₁)(R₂).

In some embodiments, the bond between X₁ and X₂ is a single bond or a double bond. In some embodiments, when the bond between X₁ and X₂ is a single bond, X₁ and X₂ are each independently C═R₁ or CR₃R₄. In some embodiments, when the bond between X₁ and X₂ is a double bond X₁ and X² are each CR₂.

In some embodiments, each occurrence of R₁ is independently selected from the group consisting of O, NH and S.

In some embodiments, each occurrence of R₂, R₃, and R₄ is independently selected from the group consisting of hydrogen, hydroxyl, carboxyl, alkyl, cycloalkyl, heterocycloalkyl, aryl, and heteroaryl.

In some embodiments, each occurrence of m is independently an integer represented by 0, 1, 2, or 5.

In some embodiments, each occurrence of p is independently an integer from 1 to 50.

In some embodiments, each occurrence of p is independently an integer from 1 to 15.

In some embodiments, each occurrence of n is independently an integer from 1 to 1000.

In some embodiments, the composition further comprises an adjuvant. In some embodiments, the adjuvant is operably linked to the compound comprising the structure of Formula (I), or a derivative or a pharmaceutical acceptable salt thereof, the nucleic acid molecule, or both.

In another aspect, the present invention relates, in part, to a composition comprising a particle, wherein the particle comprises a) a nucleic acid molecule or a fragment or variant thereof comprising a nucleotide sequence that encodes at least one antigen or a fragment or variant thereof, and b) a metabolite or a derivative or a polymer thereof.

In some embodiments, the metabolite comprises at least one selected from the group consisting of phosphoenolpyruvate, phosphoenolpyruvic acid, glucono-lactone-6-phosphate, gluconate-6-phosphase, sedoheptulose-7-phosphate, ribulose, ribulose-5-phosphate (R5P), xylulose, xylulose-5-phosphate, fructose-1,6-biphosphate (F16BP), fructose 6 phosphate (F6P), fructose-2,6-biphosphate, glycerate-2-phosphate, glucerate-3-phosphate, malate, fumarate, succinate, isocitrate, citrate, cis-aconitate, malonyl-CoA, acetyl CoA, 3-methylbutyryl CoA, 2-methylbutyryl CoA, 3-ketoacyl CoA, 3-hydroxyacyl CoA, enoyl CoA, 3-ketoacyl functionalized metabolite, 3-hydroxyacyl functionalized metabolite, enoyl functionalized metabolite, fatty acids, caprylic acid, capric acid, lauric acid, myristic acid, palmitic acid, stearic acid, arachidic acid, behenic acid, lignoceric acid, cerotic acid, myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid, docosahexaenoic acid, and any combination thereof.

In some embodiments, the composition further comprises an adjuvant. In embodiments, the adjuvant is operably linked to the metabolite or the derivative or the polymer thereof, the nucleic acid molecule, or both. Thus, in some embodiments, the particle comprises a compound comprising the structure of

or a derivative or a pharmaceutical acceptable salt thereof,
or a derivative or a pharmaceutical acceptable salt thereof,
or a derivative or a pharmaceutical acceptable salt thereof, or
or a derivative or a pharmaceutical acceptable salt thereof.

In some embodiments, each occurrence of M is independently selected from the group consisting of: Ca, Mg, Na, K, Sr, Zn, Fe, Co, and Cu.

In some embodiments, each occurrence of metabolite is independently a metabolite or derivative thereof.

In some embodiments, each occurrence of n is independently an integer from 1 to 1000.

In some embodiments, each occurrence of p is independently an integer represented by 0 or 1.

In some embodiments, the adjuvant comprises at least one selected from the group consisting of polyinosinic:polycytidylic acid (poly(I:C)) or analog thereof, muramyl dipeptide derivatives (MDP) or analog thereof, Alum and Emulsions, complete Freund's adjuvant (CFA), incomplete Freund's adjuvant (IFA), pattern recognition receptor (PRR) ligands, cyclic guanosine monophosphate-adenosine monophosphate (2′3′-cGAMP), bis-(3′-5′)-cyclic dimeric adenosine monophosphate (c-di-AMP), Rp,Rp-isomer of the 2′3′-bisphosphorothioate analog of 3′3′-cyclic adenosine monophosphate (2′3′-c-di-AM(PS)2 (Rp,Rp)), cyclic diguanylate monophosphate-stimulator of interferon genes (c-di-GMP STING)-based vaccine adjuvant, CL401, CL413, CL429, Flagellin, Imiquimod, lipopolysaccharide (LPS) from the gram-negative bacteria E. coli 0111:B4 (LPS-EB), monophosphoryl lipid A from Salmonella minnesota R595 lipopolysaccharide (MPLA-SM), synthetic monophosphoryl lipid A (MPLA), oligodeoxynucleotides (ODN) 1585, ODN 1826, ODN 2006, ODN 2395, Pam3CSK4, Resiquimod (R848), trehalose-6,6-dibehenate (TDB), and any combination thereof.

In some embodiments, the nucleic acid molecule comprises a nucleic acid molecule derived from a cancer cell, a synthetic nucleic acid molecule, or a combination thereof. In some embodiments, the nucleic acid molecule comprises an isolated mRNA or a fragment or variant thereof derived from a B16F10 cell, YUMM cell, and/or 4T1 cell.

In one embodiment, the cancer cell is a mammal cancer cell. In some embodiments, the cancer cell is a human cancer cell or canine cancer cell. In one embodiment, the cancer cell is a melanoma cell.

In some embodiments, the antigen comprises a tumor antigen or a fragment or variant thereof.

In some embodiments, the particle induces glycolysis in at least one cell of interest. In some embodiments, the cell of interest is an immune cell. In some embodiments, the immune cell is selected from the group consisting of a dendritic cell (DC), macrophage, T cell, natural killer cell, B cell, neutrophil, eosinophil, basophil, or any other immune cell, and any combination thereof.

In some embodiments, the particle is a nanoparticle or microparticle. In some embodiments, the particle has an average diameter of between about 1 μm to about 100 μm.

In some embodiments, the composition comprises calcium and phosphorous in a ratio of between about 2:about 1 to about 3:about 1.

In some embodiments, the composition further comprises a therapeutic agent. In some embodiments, the therapeutic agent comprises at least one metabolic inhibitor, metabolic pathway accelerator, peptide therapeutic, protein therapeutic, and/or enzyme to accelerate metabolism. In some embodiments, the at least one metabolic inhibitor is a glycolytic inhibitor, pentose phosphate pathway inhibitor, oxidative phosphorylation (OXPHOS) inhibitor, electron transport chain (ETC) inhibitor, and/or polyamine pathway inhibitor. In some embodiments, the at least one metabolic inhibitor comprises PFK15.

In some embodiments, the composition inhibits glycolysis in at least one cancer cell.

In some embodiments, the composition is a vaccine.

In another aspect, the present invention relates, in part, to a method of preventing or treating a metabolic inhibition of at least one cell in a subject in need thereof, the method comprising administering a therapeutically effective amount of the composition comprising at least one particle.

In another aspect, the present invention relates, in part, to a method of preventing or treating a disease or disorder in a subject in need thereof, the method comprising administering a therapeutically effective amount of the composition comprising a metabolic inhibitor, metabolic pathway accelerator, peptide therapeutic, protein therapeutic, and/or enzyme to accelerate metabolism and at least one particle. In some embodiments, the disease or disorder is cancer. For example, in some embodiments, the method comprises administering a therapeutically effective amount of the composition comprising a metabolic inhibitor and at least one particle. In some embodiments, the disease or disorder is cancer.

In some embodiments, the particle is any particle described herein.

In some embodiments, the particle comprises a) a compound comprising at least one selected from the group consisting of: i) a metabolite or a derivative or a polymer thereof; and ii) the structure of Formula (I), or a derivative or a pharmaceutical acceptable salt thereof; and b) at least one selected from the group consisting of i) a nucleic acid molecule or a fragment or variant thereof comprising a nucleotide sequence that encodes at least one antigen or a fragment or variant thereof, and ii) an antigen or a fragment or variant thereof. In some embodiments, the metabolite is any metabolite described herein.

In some embodiments, the composition induces or rescues at least one selected from the group consisting of: a glycolysis, tricarboxylic acid (TCA) cycle, polyamine pathway, pentose phosphate pathway (PPP), activation of the at least one cell, extracellular acidification rate (ECAR), oxygen consumption rate (OCR), mitochondrial respiration, release of a metabolite, pro-inflammatory response, BRAF inhibitor, tyrosine kinase inhibitor, extracellular signal-regulated kinase (ERK) inhibitor, MEK inhibitor, and cancer cell suppression.

In some embodiments, the composition a) decreases the level of at least one immune suppressive cell; b) increases the level of at least one selected from the group consisting of: a T cell, type 1 CD8+ T cell (Tc1), type 2 CD8+ T cell (Tc2), IL-17-producing CD8+ T cell (Tc17), T helper cell (Th), Th1, Th17, and effector T cell (Teff); or c) both a) and b).

In some embodiments, the composition induces or rescues glycolysis in at least one cell of interest and inhibits glycolysis in a cancer cell. In some embodiments, the at least one cell of interest is an immune cell.

In some embodiments, the composition reduces or inhibits a tumor growth; reduces a cancer cell proliferation; or a combination thereof.

In one aspect, the present invention relates, in part, to a method of inducing an immune response in a subject, comprising administering to the subject a therapeutically effective amount of at least one composition of the present invention.

In another aspect, the present invention relates, in part, to a method of inhibiting glycolysis in at least one cancer cell of interest and simultaneously inducing or rescuing glycolysis in at least one non-cancer cell of interest, comprising administering to the subject a therapeutically effective amount of at least one composition of the present invention.

In another aspect, the present invention relates, in part, to a method comprising administering at least one composition of the present invention to a cell.

In some embodiments, the cell phagocytes the particle.

In some embodiments, the cell is an immune cell. In some embodiments, the immune cell is selected from the group consisting of a dendritic cell (DC), macrophage, T cell, natural killer cell, B cell, neutrophil, eosinophil, basophil, or any other immune cell, and any combination thereof.

In another aspect, the present invention relates, in part, to a method comprising administering at least one composition of the present invention to a subject in need thereof.

In some embodiments, the subject has cancer.

In some embodiments, the composition thereof is administered directly to the subject.

In some embodiments, the method comprises the steps of: a) administering the composition to a cell, wherein the cell phagocytes the particle; and b) administering the cell comprising the phagocyted particle to the subject.

In some embodiments, the method further comprises administering a glycolytic inhibitor to the subject.

In some embodiments, the method is an adoptive T cell therapy.

### BRIEF DESCRIPTION OF THE DRAWINGS

The following detailed description of various embodiments of the present invention will be better understood when read in conjunction with the appended drawings. For the purpose of illustrating the present invention, there are shown in the drawings illustrative embodiments. It should be understood, however, that the present invention is not limited to the precise arrangements and instrumentalities of the embodiments shown in the drawings.

FIG. 1, comprising FIG. 1A through FIG. 1D, depicts a schematic representation of the formulation of the glycolic metabolite fructose 1,6 biphosphate (F16BP) into microparticles (MPs). FIG. 1A depicts a schematic representation of F16BP MPs rescuing dendritic cells (DCs) from glycolytic inhibition. FIG. 1B depicts representative dynamic light scattering (DLS) results indicating a size of 2200±300 nm of F16BP MPs. FIG. 1C depicts representative scanning electron microscopy-energy dispersive X-ray analysis (SEM-EDX) mapping demonstrating the presence of Ca and P in the microparticles. Scanning electron microscopy image indicates spherical morphology. FIG. 1D depicts representative diameter size of F16BP MPs.

FIG. 2, comprising FIG. 2A through FIG. 2J, depicts representative results demonstrating the rescue of DC glycolysis and function by F16BP MPs. FIG. 2A through FIG. 2D depict representative results demonstrating DCs treated with F16BP MPs rescued glycolysis and glycolytic capacity from glycolytic inhibition (PFK15), in vitro (n=6; One-way ANOVA Tukey's test), FIG. 2E through FIG. 2H depict representative results demonstrating DCs treated with F16BP MPs accelerate basal and maximal respiration even under glycolytic inhibition (PFK15), in vitro (n=6; One-way ANOVA Tukey's test). FIG. 2I depicts representative results demonstrating that vaccine particles induced significantly higher frequency of MHCII+CD86+ in CD11c+DCs as compared to the individual component controls of the MPs (n=6; One-way ANOVA Tukey's test). FIG. 2J depicts representative results demonstrating that F16BP MPs were able to rescue the activation of DCs even in the presence of PFK15 (PFK15 conc.=25 μM) (n=6; One-way ANOVA Tukey's test). Data represented as mean±std error.

FIG. 3, comprising FIG. 3A through FIG. 3L, depicts a representative pro-inflammatory response against melanoma, in vivo promoted by F16BP(pTRP2+PolyIC) MPs. FIG. 3A depicts a schematic representation of subcutaneous injection of vacc MPs, in vivo. FIG. 3B depicts a representative Kaplan Meir curve demonstrating significantly higher survival of mice treated with vacc MPs. FIG. 3C and FIG. 3D depict representative experimental results demonstrating that mice treated with vacc MPs had significantly higher percentage of total as well as activated DCs in the draining lymph (n=5; One-way ANOVA Tukey's test). FIG. 3E and FIG. 3F depict representative experimental results demonstrating that Mice treated with vacc MPs had significantly higher number of Tc and activated and proliferating Tc as compared to other treatment groups (n=4 or 5; One-way ANOVA Tukey's test). FIG. 3G depicts representative experimental results demonstrating no significant differences in the number of Tregs observed across treatment groups (n=5; One-way ANOVA Tukey's test). FIG. 3H through FIG. 3K depict representative results demonstrating a significantly higher number of Tc, Tc1, activated and proliferating Tc1 and Tc17 cells in mice treated with vacc MPs (n=4; One-way ANOVA Tukey's test). FIG. 3L depicts representative results demonstrating a significantly higher Tc1/Treg ratio observed in mice treated with vacc MPs as compared to the control groups (n=4; One-way ANOVA Tukey's test). Data represented as mean±std error.

FIG. 4, comprising FIG. 4A through FIG. 4E, depicts representative results demonstrating F16BP MPs rescue of DCs in adoptive cellular transfer (ACT) therapy model, in vivo. FIG. 4A depicts a schematic representation of the adoptive cellular therapy model employed. FIG. 4B and FIG. 4C depict representative results demonstrating the rescue of glycolysis and glycolytic capacity from glycolytic inhibition in mice injected with adoptively transferred DCs along with F16BP MPs were able to (PFK15), in vivo (n=6; One-way ANOVA Tukey's test). FIG. 4D and FIG. 4E depict representative results demonstrating accelerated basal and maximal respiration even under glycolytic inhibition (PFK15) in mice injected with adoptively transferred DCs along with F16BP MPs in vivo (n=6; One-way ANOVA Tukey's test). Data represented as mean±std error.

FIG. 5, comprising FIG. 5A through FIG. 5G, depicts representative results demonstrating the compatibility of F16BP MPs-based vaccines with adoptively transferred DCs and improved survival in melanoma. FIG. 5A depicts a representative Kaplan Meir curve demonstrating significantly higher survival of mice treated with adoptively transferred vacc MPs. FIG. 5B depicts representative tumor images of different treatment groups on day 16. FIG. 5C depicts representative results demonstrating significantly lower tumor weights in mice treated with adoptively transferred Vacc DCs as compared to other treatment groups, in vivo (n=3; One-way ANOVA Tukey's test). FIG. 5D and FIG. 5E depict representative results demonstrating a significantly higher total as well as activated DCs were observed in mice treated with adoptively transferred Vacc DCs as compared to other treatment groups, in vivo (n=4; One-way ANOVA Tukey's test). FIG. 5F depicts representative results demonstrating significantly higher MHCI+ activated DCs were observed in mice treated with adoptively transferred Vacc DCs as compared to other treatment groups, in vivo (n=4; One-way ANOVA Tukey's test). FIG. 5G depicts representative results demonstrating significantly higher MHCII+ activated DCs were observed in mice treated with adoptively transferred Vacc DCs as compared F16BP DCs, in vivo (n=4; One-way ANOVA Tukey's test). Data represented as mean±std error.

FIG. 6A, comprising FIG. 6A through FIG. 6G, depicts representative results demonstrating robust anti-tumor adaptive immune responses observed in DCs through adoptively transferred F16BP MPs-based vaccines. FIG. 6A and FIG. 6B depict representative results demonstrating the significant modulation of total (CD8+) as well as activated and proliferating (Ki67+CD44+ in CD8+) cytotoxic T cells observed in mice treated with adoptively transferred Vacc DCs as compared to other treatment groups, in vivo (n=4; One-way ANOVA Tukey's test). FIG. 6C through FIG. 6F depict representative results demonstrating a significantly higher total Tc1 (Tbet+ in CD8+), activated and proliferating Tc1 (Tbet+Ki67+CD44+ in CD8+), total Tc17 (ROR [symbol] T+ in CD8+), activated and proliferating Tc17 (ROR [symbol] T+Ki67+CD44+ in CD8+) observed in mice treated with adoptively transferred Vacc DCs as compared to other treatment groups, in vivo (n=4; One-way ANOVA Tukey's test). FIG. 6G depicts representative results demonstrating a significantly higher ratio of cytotoxic to regulatory T cells (Tc1/Treg) was observed in mice treated with adoptively transferred Vacc DCs as compared to other treatment groups, in vivo (n=4; One-way ANOVA Tukey's test). Data represented as mean±std error.

FIG. 7 depicts representative nuclear magnetic resonance (NMR) results indicating the presence of F16BP in the F16BP MPs.

FIG. 8 depicts representative results demonstrating the release of F16BP over time and that F16BP MPs released F16BP for 6 hours in a sustained manner. Data represented as mean±std error.

FIG. 9 depicts representative results demonstrating the generation of microparticles of other control metabolites, such as ribulose 5 phosphate (R5P), phosphoenolpyruvic acid, and fructose 6 phosphate (F6P).

FIG. 10 depicts representative confocal microscopy images that indicate the phagocytosis of F16BP MPs by DCs. Scale bar=100 μm FIG. 11 depicts representative seahorse assay measurements for DCs treated with different treatment groups. (n=5; One-way ANOVA Tukey's test). Data represented as mean±std error.

FIG. 12 depicts representative results demonstrating the generation of microparticles using F16BP, poly I.C, and pTRP2.

FIG. 13 depicts representative results of a proliferation assay using MTT demonstrating that PFK15 prevented growth of YUMM1.1 cancer cells (n=6). Data represented as mean±std error.

FIG. 14 depicts representative results demonstrating individual tumor growth in mice treated with various treatment groups along with representative tumor images on day 35.

FIG. 15 depicts representative results demonstrating subcutaneously injected Vacc MPs modulated adaptive immune responses in tumors, in vivo (n=5; One-way ANOVA Tukey's test). Data represented as mean±std error.

FIG. 16 depicts a schematic representation of the study design for adoptive cell therapy model.

FIG. 17 depicts representative results of a proliferation assay using MTT demonstrating that PFK15 prevented growth of YUMM1.1 cancer cells (n=6). Data represented as mean±std error.

FIG. 18 depicts representative results demonstrating the individual tumor growth in mice treated with various treatment groups along with representative tumor images on day 35.

FIG. 19 depicts representative results demonstrating adoptively transferred Vacc DCs modulated adaptive immune responses in tumors, in vivo (n=4; One-way ANOVA Tukey's test). Data represented as mean±std error.

FIG. 20 depicts representative results demonstrating adoptively transferred Vacc DCs modulated innate immune responses in spleen of mice treated with different groups, in vivo (n=4; One-way ANOVA Tukey's test). Data represented as mean±std error.

FIG. 21 depicts representative results demonstrating no significant differences were observed in innate immune responses in adoptively transferred Vacc DCs as compared to other treatment groups in the cervical lymph nodes, in vivo (n=4; One-way ANOVA Tukey's test). Data represented as mean±std error.

FIG. 22 depicts representative results demonstrating adoptively transferred Vacc DCs modulated adaptive immune responses (helper T cell) in spleen of mice treated with different groups, in vivo (n=4; One-way ANOVA Tukey's test). Data represented as mean±std error.

FIG. 23 depicts representative results demonstrating adoptively transferred Vacc DCs modulated adaptive immune responses (cytotoxic T cell) in spleen of mice treated with different groups, in vivo (n=4; One-way ANOVA Tukey's test). Data represented as mean±std error.

### DETAILED DESCRIPTION

The present invention is based, in part, on the unexpected discovery that compositions comprising fructose-1,6-biphosphate (F16BP) particles as a metabolite, PFK15 as a glycolytic inhibitor, and a peptide or an mRNA simultaneously inhibited glycolysis in cancer cells while rescuing glycolysis in DCs and modulating the immune function of DCs. Thus, in various aspects, the present invention provides a particle comprising a) a metabolite or a derivative or a polymer thereof and b) an antigen or a fragment or variant thereof and/or a nucleic acid molecule or a fragment or variant thereof comprising a nucleotide sequence that encodes at least one antigen or a fragment or variant thereof. In other aspects, the present invention provides a particle comprising a) a compound comprising the structure of Formula (I), or a derivative or a pharmaceutical acceptable salt thereof and b) an antigen or a fragment or variant thereof and/or a nucleic acid molecule or a fragment or variant thereof comprising a nucleotide sequence that encodes at least one antigen or a fragment or variant thereof. In some embodiments, the nucleic acid molecule comprises a nucleic acid molecule derived from a cancer cell (e.g., an isolated mRNA or a fragment or variant thereof derived from a B16F10 cell, YUMM cell, or 4T1 cell), a synthetic nucleic acid molecule, or a combination thereof.

In some aspects, the present invention provides a composition comprising at least one particle of the present invention. In some embodiments, the composition further comprises an adjuvant.

The present invention further provides a method for inducing an immune response, preventing or treating a metabolic inhibition in the presence of one or more metabolic inhibitors, preventing or treating a disease or disorder, such as cancer, and/or adoptive T cell therapy using at least one particle or composition described herein. The present invention also relates to a method for inducing glycolysis, tricarboxylic acid (TCA) cycle, polyamine pathway, pentose phosphate pathway (PPP), activation of at least one cell (e.g., immune cell), extracellular acidification rate (ECAR), oxygen consumption rate (OCR), mitochondrial respiration, release of a metabolite, pro-inflammatory response, BRAF inhibitor, tyrosine kinase inhibitor, extracellular signal-regulated kinase (ERK) inhibitor, MEK inhibitor, cancer cell suppression, and/or increase in the level of immune cells in the presence of one or more metabolic inhibitors, metabolic pathway accelerators, peptide therapeutics, protein therapeutics, and/or enzymes to accelerate metabolism using at least one particle or composition described herein. The present invention further relates to methods for reducing or inhibiting a tumor growth and/or reducing a cancer cell proliferation using at least one particle or composition described herein. The present invention also provides methods of treating a disease or disorder by combining immunotherapy with metabolic inhibition. The present invention additionally provides kits that find use in the practice of the methods of the invention.

### Definitions

As used herein, each of the following terms has the meaning associated with it in this section. Unless defined elsewhere, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent depending on the context in which it is used. As used herein when referring to a measurable value such as an amount, a temporal duration, and the like, the term “about” is meant to encompass variations of 20% or ±10%, more preferably ±5%, even more preferably ±1%, and still more preferably ±0.1% from the specified value, as such variations are appropriate to perform the disclosed methods.

The term “derivative” refers to a small molecule that differs in structure from the reference molecule, but may retain or enhance the essential properties of the reference molecule and may have additional properties. A derivative may change its interaction with certain other molecules relative to the reference molecule. A derivative molecule may also include a salt, an adduct, tautomer, isomer, or other variant of the reference molecule.

The term “tautomers” are constitutional isomers of organic compounds that readily interconvert by a chemical process (tautomerization).

The term “isomers” or “stereoisomers” refers to compounds, which have identical chemical constitution, but differ with regard to the arrangement of the atoms or groups in space.

As used herein, “alkyl” refers to a linear or branched chain fully saturated (no double or triple bonds) hydrocarbon (all carbon) group. An alkyl group of this invention may comprise from 1-20 carbon atoms, that is, “m”=1 and “n”=20, designated as a “C₁ to C₂₀ alkyl.” In one embodiment, “m”=1 and “n”=12 (C₁ to C₁₂ alkyl). In other embodiments, that “m”=1 and “n”=6 (C₁ to C₆ alkyl). Examples of alkyl groups include, without limitation, methyl, ethyl, n-propyl, isopropyl, n-butyl, iso-butyl, sec-butyl, tert-butyl, amyl, tert-amyl, hexyl, heptyl, octyl, nonyl, decyl, undecyl, and dodecyl.

An alkyl group of this invention may be substituted or unsubstituted. When substituted, the substituent group(s) is(are) one or more group(s) independently selected from cycloalkyl, aryl, heteroaryl, heteroalicyclyl, hydroxy, alkoxy, aryloxy, mercapto, alkylthio, arylthio, cyano, halo, oxo, carbonyl, thiocarbonyl, 0-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, trihalomethanesulfonyl, —NRaRb, protected hydroxyl, protected amino, protected carboxy, and protected amido groups.

Examples of substituted alkyl groups include, without limitation, 2-oxo-prop-1-yl, 3-oxo-but-1-yl, cyanomethyl, nitromethyl, chloromethyl, hydroxymethyl, tetrahydropyranyloxymethyl, m-trityloxymethyl, propionyloxymethyl, aminomethyl, carboxymethyl, allyloxycarbonylmethyl, allyloxycarbonylaminomethyl, methoxymethyl, ethoxymethyl, t-butoxymethyl, acetoxymethyl, chloromethyl, bromomethyl, iodomethyl, trifluoromethyl, 6-hydroxyhexyl, 2,4-dichlorobutyl, 2-aminopropyl, 1-chloroethyl, 2-chloroethyl, 1-bromoethyl, 2-chloroethyl, 1-fluoroethyl, 2-fluoroethyl, 1-iodoethyl, 2-iodoethyl, 1-chloropropyl, 2-chloropropyl, 3-chloropropyl, 1-bromopropyl, 2-bromopropyl, 3-bromopropyl, 1-fluoropropyl, 2-fluoropropyl, 3-fluoropropyl, 1-iodopropyl, 2-iodopropyl, 3-iodopropyl, 2-aminoethyl, 1-aminoethyl, N-benzoyl-2-aminoethyl, Nacetyl-2-aminoethyl, N-benzoyl-1-aminoethyl, and N-acetyl-1-aminoethyl.

As used herein, “aryl” refers to a carbocyclic (all carbon) ring or two or more fused rings (rings that share two adjacent carbon atoms) that have a fully delocalized pi-electron system. Examples of aryl groups include, but are not limited to, benzene, and substituted benzene, such as toluene, aniline, xylene, and the like, naphthalene and substituted naphthalene, and azulene.

“Pharmaceutically acceptable” refers to those properties and/or substances which are acceptable to the subject from a pharmacological/toxicological point of view and to the manufacturing pharmaceutical chemist from a physical/chemical point of view regarding composition, formulation, stability, subject acceptance and bioavailability. “Pharmaceutically acceptable carrier” refers to a medium that does not interfere with the effectiveness of the biological activity of the active ingredient(s) and is not toxic to the host to which it is administered.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the subject. Some examples of materials that may serve as pharmaceutically acceptable carriers include: sugars, such as lactose, glucose, and sucrose; starches, such as corn starch and potato starch; cellulose, and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the subject. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art.

The term “pharmaceutically acceptable salt” refers to any pharmaceutically acceptable salt, which upon administration to the subject is capable of providing (directly or indirectly) a compound as described herein. Such salts are, for example, acid addition salts with physiologically acceptable organic or inorganic acids. Examples of the acid addition salts include mineral acid addition salts such as, for example, hydrochloride, hydrobromide, hydroiodide, sulphate, nitrate, phosphate, and organic acid addition salts such as, for example, acetate, trifluoroacetate, maleate, fumarate, citrate, oxalate, succinate, tartrate, malate, mandelate, methane sulphonate, and p-toluenesulphonate. Examples of the alkali addition salts include inorganic salts such as, for example, sodium, potassium, calcium and ammonium salts, and organic alkali salts such as, for example, ethylenediamine, ethanolamine, N,N-dialkylenethanolamine, triethanolamine, and basic amino acids salts. However, it will be appreciated that non-pharmaceutically acceptable salts also fall within the scope of the invention since those may be useful in the preparation of pharmaceutically acceptable salts. Procedures for salt formation are conventional in the art.

As used herein, the term “pharmaceutical composition” refers to a mixture of at least one compound of the invention with other chemical components and entities, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, oral, aerosol, parenteral, ophthalmic, pulmonary and topical administration.

As used herein, the terms “therapeutic compound”, “therapeutic agent”, “drug”, “active pharmaceutical”, and “active pharmaceutical ingredient” are used interchangeably to refer to chemical entities that display certain pharmacological effects in a body and are administered for such purpose. Non-limiting examples of therapeutic agents include, but are not limited to, antibiotics, analgesics, vaccines, anticonvulsants; anti-diabetic agents, antifungal agents, antineoplastic agents, anti-parkinsonian agents, anti-rheumatic agents, appetite suppressants, biological response modifiers, cardiovascular agents, central nervous system stimulants, contraceptive agents, dietary supplements, vitamins, minerals, lipids, saccharides, metals, metabolites, amino acids (and precursors), nucleic acids and precursors, contrast agents, diagnostic agents, dopamine receptor agonists, erectile dysfunction agents, fertility agents, gastrointestinal agents, hormones, immunomodulators, antihypercalcemia agents, mast cell stabilizers, muscle relaxants, nutritional agents, ophthalmic agents, osteoporosis agents, psychotherapeutic agents, parasympathomimetic agents, parasympatholytic agents, respiratory agents, sedative hypnotic agents, skin and mucous membrane agents, smoking cessation agents, steroids, sympatholytic agents, urinary tract agents, uterine relaxants, vaginal agents, vasodilator, anti-hypertensive, hyperthyroids, anti-hyperthyroids, anti-asthmatics and vertigo agents. In certain embodiments, the one or more therapeutic agents are water-soluble, poorly water-soluble drug or a drug with a low, medium or high melting point. The therapeutic agents may be provided with or without a stabilizing salt or salts.

Some examples of active ingredients suitable for use in the pharmaceutical formulations and methods of the present invention include: hydrophilic, lipophilic, amphiphilic or hydrophobic, and that can be solubilized, dispersed, or partially solubilized and dispersed, on or about the nanoparticle cluster. The active agent-nanoparticle cluster combination may be coated further to encapsulate the agent-nanoparticle cluster combination and may be directed to a target by functionalizing the nanoparticle cluster with, e.g., aptamers and/or antibodies. Alternatively, an active ingredient may also be provided separately from the solid pharmaceutical composition, such as for co-administration. Such active ingredients can be any compound or mixture of compounds having therapeutic or other value when administered to an animal, particularly to a mammal, such as drugs, nutrients, cosmeceuticals, nutraceuticals, diagnostic agents, nutritional agents, and the like. The active agents described herein may be found in their native state, however, they will generally be provided in the form of a salt. The active agents described herein include their isomers, analogs and derivatives.

The term “antibody”, as used herein, refers to an immunoglobulin molecule which is able to specifically bind to a specific epitope of an antigen. Antibodies can be intact immunoglobulins derived from natural sources, or from recombinant sources and can be immunoreactive portions of intact immunoglobulins. The antibodies in the present invention may exist in a variety of forms including, for example, polyclonal antibodies, monoclonal antibodies, multiple chain antibodies, intact immunoglobulins, synthetic antibodies, recombinant antibodies, intracellular antibodies (“intrabodies”), Fv, Fab, Fab′, F(ab)2 and F(ab′)2, as well as single chain antibodies (scFv), heavy chain antibodies, such as camelid antibodies, and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antibody fragment” refers to at least one portion of an intact antibody, or recombinant variants thereof, and refers to the antigen binding domain, e.g., an antigenic determining variable region of an intact antibody, that is sufficient to confer recognition and specific binding of the antibody fragment to a target, such as an antigen.

By the term “synthetic antibody” as used herein, is meant an antibody which is generated using recombinant DNA technology, such as, for example, an antibody expressed by a bacteriophage. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

A “humanized antibody” refers to a type of engineered antibody having its CDRs derived from a non-human donor immunoglobulin, the remaining immunoglobulin-derived parts of the molecule being derived from one (or more) human immunoglobulin(s). In addition, framework support residues may be altered to preserve binding affinity (see, e.g., 1989, Queen et al., Proc. Natl. Acad Sci USA, 86:10029-10032; 1991, Hodgson et al., Bio/Technology, 9:421). A suitable human acceptor antibody may be one selected from a conventional database, e.g., the KABAT database, Los Alamos database, and Swiss Protein database, by homology to the nucleotide and amino acid sequences of the donor antibody. A human antibody characterized by a homology to the framework regions of the donor antibody (on an amino acid basis) may be suitable to provide a heavy chain constant region and/or a heavy chain variable framework region for insertion of the donor CDRs. A suitable acceptor antibody capable of donating light chain constant or variable framework regions may be selected in a similar manner. It should be noted that the acceptor antibody heavy and light chains are not required to originate from the same acceptor antibody. The prior art describes several ways of producing such humanized antibodies (see for example EP-A-0239400 and EP-A-054951).

A “chimeric antibody” refers to a type of engineered antibody which contains a naturally-occurring variable region (light chain and heavy chains) derived from a donor antibody in association with light and heavy chain constant regions derived from an acceptor antibody.

The term “donor antibody” refers to an antibody (monoclonal, and/or recombinant) which contributes the amino acid sequences of its variable regions, CDRs, or other functional fragments or analogs thereof to a first immunoglobulin partner, so as to provide the altered immunoglobulin coding region and resulting expressed altered antibody with the antigenic specificity and neutralizing activity characteristic of the donor antibody.

The term “acceptor antibody” refers to an antibody (monoclonal and/or recombinant) heterologous to the donor antibody, which contributes all (or any portion, but in some embodiments all) of the amino acid sequences encoding its heavy and/or light chain framework regions and/or its heavy and/or light chain constant regions to the first immunoglobulin partner. In certain embodiments a human antibody is the acceptor antibody.

By the term “recombinant antibody” as used herein, is meant an antibody which is generated using recombinant DNA technology, such as, for example, an antibody expressed by a bacteriophage or yeast expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

An “antibody heavy chain,” as used herein, refers to the larger of the two types of polypeptide chains present in antibody molecules in their naturally occurring conformations, and which normally determines the class to which the antibody belongs.

An “antibody light chain,” as used herein, refers to the smaller of the two types of polypeptide chains present in antibody molecules in their naturally occurring conformations. Kappa (κ) and lambda (λ) light chains refer to the two major antibody light chain isotypes.

“CDRs” are defined as the complementarity determining region amino acid sequences of an antibody which are the hypervariable regions of immunoglobulin heavy and light chains. See, e.g., Kabat et al., Sequences of Proteins of Immunological Interest, 4th Ed., U.S. Department of Health and Human Services, National Institutes of Health (1987). There are three heavy chain and three light chain CDRs (or CDR regions) in the variable portion of an immunoglobulin. Thus, “CDRs” as used herein refers to all three heavy chain CDRs, or all three light chain CDRs (or both all heavy and all light chain CDRs, if appropriate). The structure and protein folding of the antibody may mean that other residues are considered part of the antigen binding region and would be understood to be so by a skilled person. See for example Chothia et al., (1989) Conformations of immunoglobulin hypervariable regions; Nature 342, p 877-883.

As used herein, the term “stabilizers” refers to either, or both, primary particle and/or secondary stabilizers, which may be polymers or other small molecules. Non-limiting examples of primary particle and/or secondary stabilizers for use with the present invention include, e.g., starch, modified starch, and starch derivatives, gums, including but not limited to polymers, polypeptides, albumin, amino acids, thiols, amines, carboxylic acid and combinations or derivatives thereof. Other examples include xanthan gum, alginic acid, other alginates, benitoniite, veegum, agar, guar, locust bean gum, gum arabic, quince psyllium, flax seed, okra gum, arabinoglactin, pectin, tragacanth, scleroglucan, dextran, amylose, amylopectin, dextrin, etc., cross-linked polyvinylpyrrolidone, ion-exchange resins, potassium polymethacrylate, carrageenan (and derivatives), gum karaya and biosynthetic gum. Other examples of useful primary particle and/or secondary stabilizers include polymers such as: polycarbonates (linear polyesters of carbonic acid); microporous materials (bisphenol, a microporous poly(vinylchloride), micro-porous polyamides, microporous modacrylic copolymers, microporous styrene-acrylic and its copolymers); porous polysulfones, halogenated poly(vinylidene), polychloroethers, acetal polymers, polyesters prepared by esterification of a dicarboxylic acid or anhydride with an alkylene polyol, poly(alkylenesulfides), phenolics, polyesters, asymmetric porous polymers, cross-linked olefin polymers, hydrophilic microporous homopolymers, copolymers or interpolymers having a reduced bulk density, and other similar materials, poly(urethane), cross-linked chain-extended poly(urethane), poly(mides), poly(benzimidazoles), collodion, regenerated proteins, semi-solid cross-linked poly(vinylpyrrolidone).

As used herein, the terms “targeting domain”, “targeting moiety”, or “targeting group” are used interchangeably and refer to all molecules capable of specifically binding to a particular target molecule and forming a bound complex as described above. Thus, the ligand and its corresponding target molecule form a specific binding pair.

As used herein, the term “specific binding” refers to that binding which occurs between such paired species as enzyme/substrate, receptor/agonist, antibody/antigen, and lectin/carbohydrate which may be mediated by covalent or non-covalent interactions or a combination of covalent and non-covalent interactions. When the interaction of the two species produces a non-covalently bound complex, the binding which occurs is typically electrostatic, hydrogen-bonding, or the result of lipophilic interactions. Accordingly, “specific binding” occurs between a paired species where there is interaction between the two which produces a bound complex having the characteristics of an antibody/antigen or enzyme/substrate interaction. In particular, the specific binding is characterized by the binding of one member of a pair to a particular species and to no other species within the family of compounds to which the corresponding member of the binding member belongs. Thus, for example, an antibody preferably binds to a single epitope and to no other epitope within the family of proteins.

The term “specifically binds”, as used herein with respect to an antibody, is meant for an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “associated” as used herein, or “operably linked” refers to a juxtaposition between a regulatory and a coding sequence, wherein the components so described are in a relationship permitting them to function in their intended manner. A regulatory sequence “operably linked” to a coding sequence is ligated in such a way that expression of the coding sequence is achieved under conditions compatible with the control sequences. In case the regulatory sequence is a promoter, it would be known to a skilled person that a double-stranded nucleic acid is preferable. The associated nucleic acid encompasses heterologous nucleic acids. Heterologous nucleic acids refer to nucleic acids derived from a separate genetic source, for example nucleic acids that originate from within the cell but that are not naturally located in the cell, or that are located in a different chromosomal site of the cell. Heterologous nucleic acids may also be derived from other species and may be introduced as a transgene, for example, by transformation. This transgene may be substantially modified from its native form in composition and/or genomic environment through deliberate human manipulation. The term “operably linked” also refers to the juxtaposition between two or more molecules. For example, an amino acid sequence is operably linked to one or more compounds of the present invention. As such, the term “operably linked” refers to one or more covalent bonds, non-covalent bonds, ionic bonds, and/or van der Waal force between two or more molecules.

As used herein, the terms “peptide”, “polypeptide”, and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or any combination thereof.

As used herein, the terms “amino acid”, “amino acidic monomer”, or “amino acid residue” refer to any of the twenty naturally occurring amino acids, synthetic amino acids with unnatural side chains, and including both D and L optical isomers.

“Nucleic acid” or “oligonucleotide” or “polynucleotide” as used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

“Substantially complementary” as used herein may mean that a first sequence is at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or that the two sequences hybridize under stringent hybridization conditions.

“Substantially identical” as used herein may mean that a first and second sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%,or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or more nucleotides or amino acids, or with respect to nucleic acids, if the first sequence is substantially complementary to the complement of the second sequence.

“Variant” used herein with respect to a nucleic acid may mean (i) a portion or fragment of a referenced nucleotide sequence; (ii) the complement of a referenced nucleotide sequence or portion thereof, (iii) a nucleic acid that is substantially identical to a referenced nucleic acid or the complement thereof, or (iv) a nucleic acid that hybridizes under stringent conditions to the referenced nucleic acid, complement thereof, or a sequences substantially identical thereto.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of 2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within ±2 of each other. Both the hyrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

A variant may be a nucleic acid sequence that is substantially identical over the full length of the full gene sequence or a fragment thereof. The nucleic acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the gene sequence or a fragment thereof. A variant may be an amino acid sequence that is substantially identical over the full length of the amino acid sequence or fragment thereof. The amino acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof.

“Isolated” means altered or removed from the natural state. For example, a peptide naturally present in a living animal is not “isolated,” but the same peptide partially or completely separated from the coexisting materials of its natural state is “isolated.” An isolated protein can exist in substantially purified form, or can exist in a non-native environment such as, for example, a host cell.

As used herein, the term “fragment,” as applied to a nucleic acid, refers to a subsequence of a larger nucleic acid. As used herein, the term “fragment,” as applied to a protein or peptide, refers to a subsequence of a larger protein or peptide.

The term “functionally equivalent” as used herein refers to a polypeptide according to the invention that retains at least one biological function or activity of the specific amino acid sequence of either the first or second peptide.

As used herein, the term “immune response” includes T cell mediated and/or B cell mediated immune responses that are influenced by modulation of T cell co-stimulation. The term immune response further includes immune responses that are indirectly effected by T cell activation such as antibody production (humoral responses) and the activation of cytokine responsive cells such as macrophages.

The terms “cells” and “population of cells” are used interchangeably and refer to a plurality of cells, i.e., more than one cell. The population may be a pure population comprising one cell type. Alternatively, the population may comprise more than one cell type. In the present invention, there is no limit on the number of cell types that a cell population may comprise.

As used herein, the term “immune cell” includes cells that are of haematopoietic origin and that play a role in the immune response. Immune cells include lymphocytes, such as B cells and T cells; natural killer cells; myeloid cells, such as monocytes, macrophages, dendritic cells, eosinophils, mast cells, basophils, and granulocytes.

As used herein, the term “T cell” refers to a lymphocyte (e.g., white blood cell) that functions in cell-mediated immunity. In some embodiments, the presence of a T cell receptor (TCR) on the cell surface distinguishes T cells from other lymphocytes. As is known in the art, T cells typically do not present antigens, and rely on other lymphocytes (e.g., natural killer cells and B cells) to aid in antigen presentation. Types of T cells include: T helper cells (TH cells), Memory T cells (Tcm, Tem, or Temra), Regulatory T cells (Treg), Cytotoxic T cells (CTLs), Natural killer T cells (NK cells), gamma delta T cells, and Mucosal associated invariant T cells (MAIT). As used herein, the term “T cell” includes CD4+ T cells and CD8+ T cells. The term T cell also includes both T helper 1 type T cells and T helper 2 type T cells and also Th-IL 17 cells.

As used herein, the term “dendritic cell” or “dendritic cells” (DC) refers to a dendritic cell or cells in its broadest context and includes any DC that is capable of antigen presentation. The term includes all DC that initiate an immune response and/or present an antigen to T lymphocytes and/or provide T-cells with any other activation signal required for stimulation of an immune response. Reference herein to “DC” should be read as including reference to cells exhibiting dendritic cell morphology, phenotype or functional activity and to mutants or variants thereof. The morphological features of dendritic cells may include, but are not limited to, long cytoplasmic processes or large cells with multiple fine dendrites. Phenotypic characteristics may include, but are not limited to, expression of one or more of MHC class I molecules, MHC class II molecules, CD11c, B220, CD8-alpha, CD1, CD4.

As used herein, the term “antigen-presenting cell” or “antigen-presenting cells” or its abbreviation “APC” or “APCs” refers to a cell or cells capable of endocytotic adsorption, processing and presenting of an antigen. The term includes professional antigen presenting cells for example; B lymphocytes, monocytes, dendritic cells (DCs) and Langerhans cells, as well as other antigen presenting cells such as keratinocytes, endothelial cells, glial cells, fibroblasts and oligodendrocytes. The term “antigen presenting” means the display of antigen as peptide fragments bound to MHC molecules, on the cell surface. Many different kinds of cells may function as APCs including, for example, macrophages, B cells, follicular dendritic cells and dendritic cells.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human. In various embodiments, the subject is a human subject, and may be of any race, ethnicity, sex, and age.

The terms “effective amount” and “pharmaceutically effective amount” refer to a sufficient amount of an agent to provide the desired biological result. That result can be reduction and/or alleviation of a sign, symptom, or cause of a disease or disorder, or any other desired alteration of a biological system. An appropriate effective amount in any individual case may be determined by one of ordinary skill in the art using routine experimentation.

A “therapeutically effective amount” refers to that amount which provides a therapeutic effect for a given condition and administration regimen. In particular, “therapeutically effective amount” means an amount that is effective to prevent, alleviate or ameliorate symptoms of the disease or prolong the survival of the subject being treated, which may be a human or non-human animal. Determination of a therapeutically effective amount is within the skill of the person skilled in the art.

A “therapeutic” treatment is a treatment administered to a subject who exhibits signs or symptoms of a disease or disorder, for the purpose of diminishing or eliminating those signs or symptoms.

As used herein, “treating a disease or disorder” means reducing the severity and/or frequency with which a sign or symptom of the disease or disorder is experienced by a subject.

As used herein, a “prophylactic” or “preventive” treatment is a treatment administered to a subject who does not exhibit the signs or symptoms of a disease or disorder or exhibits only early signs or symptoms of the disease or disorder for the purpose of decreasing the risk of developing additional or more severe signs of symptoms associated with the disease or disorder.

A disease or disorder is “alleviated” if the severity of a sign or symptom of the disease or disorder, the frequency with which such a sign or symptom is experienced by a subject, or both, is reduced.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Cancer,” as used herein, refers to the abnormal growth or division of cells. Generally, the growth and/or life span of a cancer cell exceeds, and is not coordinated with, that of the normal cells and tissues around it. Cancers may be benign, pre-malignant or malignant. Cancer occurs in a variety of cells and tissues, including, but not limited to, the oral cavity (e.g., mouth, tongue, pharynx, etc.), digestive system (e.g., esophagus, stomach, small intestine, colon, rectum, liver, bile duct, gall bladder, pancreas, etc.), respiratory system (e.g., larynx, lung, bronchus, etc.), bones, joints, skin (e.g., basal cell, squamous cell, meningioma, etc.), breast, genital system, (e.g., uterus, ovary, prostate, testis, etc.), urinary system (e.g., bladder, kidney, ureter, etc.), eye, nervous system (e.g., brain, etc.), endocrine system (e.g., thyroid, etc.), soft tissues (e.g., muscle, fat, etc.), and hematopoietic system (e.g., lymphoma, myeloma, leukemia, acute lymphocytic leukemia, chronic lymphocytic leukemia, acute myeloid leukemia, chronic myeloid leukemia, etc.).

The term “inhibit,” as used herein, means to suppress or block an activity or function by at least about ten percent relative to a control value. In various embodiments, the activity is suppressed or blocked by at least about 50%, at least about 55%, at least about 60%, at least about 65%, at least about 70%, at least about 75%, at least about 80%, at least about 85%, at least about 90%, or at least about 95%, as compared with a comparator value. “Instructional material”, as that term is used herein, includes a publication, a recording, a diagram, or any other medium of expression which can be used to communicate the usefulness of the nanoparticles or compositions thereof of the present invention in the kit for modulating function of cells, modulating a metabolic inhibition of cells, modulating an immunoresponse, and/or preventing or treating the various diseases or disorders recited herein. Optionally, or alternately, the instructional material may describe one or more methods of for modulating function of cells, modulating a metabolic inhibition of cells, modulating an immunoresponse, and/or preventing or treating the various diseases or disorders in a cell or a tissue of a subject. The instructional material of the kit may, for example, be affixed to a container that contains one or more components of the invention or be shipped together with a container that contains the one or more components of the invention. Alternatively, the instructional material may be shipped separately from the container with the intention that the recipient uses the instructional material and the components cooperatively.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range, such as from 1 to 6, should be considered to have specifically disclosed subranges, such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention is based, in part, on the unexpected discovery that compositions comprising fructose-1,6-biphosphate (F16BP) particles as a metabolite, PFK15 as a glycolytic inhibitor, and a peptide or an mRNA simultaneously inhibited glycolysis in cancer cells while rescuing glycolysis in DCs and modulating the immune function of DCs. Thus, in various aspects, the present invention provides a particle comprising a) a metabolite or a derivative or a polymer thereof and b) an antigen or a fragment or variant thereof and/or a nucleic acid molecule or a fragment or variant thereof comprising a nucleotide sequence that encodes at least one antigen or a fragment or variant thereof. In other aspects, the present invention provides a particle comprising a) a compound comprising the structure of Formula (I), or a derivative or a pharmaceutical acceptable salt thereof and b) an antigen or a fragment or variant thereof and/or a nucleic acid molecule or a fragment or variant thereof comprising a nucleotide sequence that encodes at least one antigen or a fragment or variant thereof. In some embodiments, the nucleic acid molecule comprises a nucleic acid molecule derived from a cancer cell (e.g., an isolated mRNA or a fragment or variant thereof derived from a B16F10 cell, YUMM cell, or 4T1 cell), a synthetic nucleic acid molecule, or a combination thereof.

In some aspects, the present invention provides a composition comprising at least one particle of the present invention. In some embodiments, the composition further comprises an adjuvant.

The present invention further provides a method for inducing an immune response, preventing or treating a metabolic inhibition in the presence of one or more metabolic inhibitors, preventing or treating a disease or disorder, such as cancer, and/or adoptive T cell therapy using at least one particle or composition described herein. The present invention also relates to a method for inducing glycolysis, TCA cycle, polyamine pathway, PPP, activation of at least one cell (e.g., immune cell), ECAR, OCR, mitochondrial respiration, release of a metabolite, pro-inflammatory response, BRAF inhibitor, tyrosine kinase inhibitor, ERK inhibitor, MEK inhibitor, cancer cell suppression, and/or increase in the level of immune cells in the presence of one or more metabolic inhibitors, metabolic pathway accelerators, peptide therapeutics, protein therapeutics, and/or enzymes to accelerate metabolism using at least one particle or composition described herein. The present invention further relates to methods for reducing or inhibiting a tumor growth and/or reducing a cancer cell proliferation using at least one particle or composition described herein. The present invention also provides methods of treating a disease or disorder by combining immunotherapy with metabolic inhibition. The present invention additionally provides kits that find use in the practice of the methods of the invention.

### Particles

In one aspect, the invention provides a particle comprising a) a metabolite or a derivative or a polymer thereof and b) an antigen or a fragment or variant thereof and/or a nucleic acid molecule or a fragment or variant thereof comprising a nucleotide sequence that encodes at least one antigen or a fragment or variant thereof.

In one embodiment, the polymer modulates the function of an immune cell. In one embodiment, the polymer induces an immune response in a subject. In one embodiment, the polymer prevents a metabolic inhibition. In one embodiment, the polymer treats a metabolic inhibition. In one embodiment, the polymer induces an activation of a cell. In one embodiment, the polymer induces an activation of an immune cell. In one embodiment, the polymer induces an activation of a dendritic cell. In one embodiment, the polymer induces glycolysis. In one embodiment, the polymer induces a TCA cycle. In one embodiment, the polymer induces a polyamine pathway. In one embodiment, the polymer induces a PPP. In one embodiment, the polymer induces an ECAR. In one embodiment, the polymer induces an OCR. In one embodiment, the polymer induces a mitochondrial respiration. In one embodiment, the polymer induces a release of a metabolite. In one embodiment, the polymer induces a pro-inflammatory response. In one embodiment, the polymer induces one or more BRAF inhibitors. In one embodiment, the polymer induces cancer cell suppression. In one embodiment, the polymer induces one or more tyrosine kinase inhibitors. In one embodiment, the polymer induces one or more ERK inhibitors. In one embodiment, the polymer induces one or more MEK inhibitors. In one embodiment, the polymer reduces cancer cell proliferation. In some embodiments, the polymer increases the level of at least one T cell, type 1 CD8+ T cell (Tc1), type 2 CD8+ T cell (Tc2), IL-17-producing CD8+ T cell (Tc17), T helper cell (Th), Th1, Th17, or effector T cell (Teff). In some embodiments, the polymer decreases the level of at least one immune suppressive cell, Th2, regulatory T cell (Treg), Foxp3, Foxp3+ cell, GATA3, or GATA3+ cell.

In one embodiment, the polymer modulates the function of an immune cell in the presence of one or more metabolic inhibitors, metabolic pathway accelerators, peptide therapeutics, protein therapeutics, and/or enzymes to accelerate metabolism. For example, in one embodiment, the polymer induces an immune response in a subject in the presence of one or more metabolic inhibitors. In one embodiment, the polymer prevents a metabolic inhibition in the presence of one or more metabolic inhibitors. In one embodiment, the polymer treats a metabolic inhibition in the presence of one or more metabolic inhibitors. In one embodiment, the polymer induces an activation of a cell in the presence of one or more metabolic inhibitors. In one embodiment, the polymer induces an activation of an immune cell in the presence of one or more metabolic inhibitors. In one embodiment, the polymer induces an activation of a dendritic cell in the presence of one or more metabolic inhibitors.

In one embodiment, the polymer induces glycolysis in the presence of one or more metabolic inhibitors. In one embodiment, the polymer induces a TCA cycle in the presence of one or more metabolic inhibitors. In one embodiment, the polymer induces a polyamine pathway in the presence of one or more metabolic inhibitors. In one embodiment, the polymer induces a PPP in the presence of one or more metabolic inhibitors. In one embodiment, the polymer induces an ECAR in the presence of one or more metabolic inhibitors. In one embodiment, the polymer induces an OCR in the presence of one or more metabolic inhibitors. In one embodiment, the polymer induces a mitochondrial respiration in the presence of one or more metabolic inhibitors. In one embodiment, the polymer induces a release of a metabolite in the presence of one or more metabolic inhibitors. In one embodiment, the polymer induces a pro-inflammatory response in the presence of one or more metabolic inhibitors. In one embodiment, the polymer induces one or more BRAF inhibitors in the presence of one or more metabolic inhibitors. In one embodiment, the polymer induces one or more tyrosine kinase inhibitors in the presence of one or more metabolic inhibitors. In one embodiment, the polymer induces one or more ERK inhibitors in the presence of one or more metabolic inhibitors. In one embodiment, the polymer induces one or more MEK inhibitors in the presence of one or more metabolic inhibitors. In one embodiment, the polymer induces a cancer cell suppression in the presence of one or more metabolic inhibitors. In one embodiment, the polymer reduces cancer cell proliferation in the presence of one or more metabolic inhibitors. In some embodiments, the polymer increases the level of at least one T cell, Tc1, Tc2, Tc17, Th, Th1, Th17, or Teff in the presence of one or more metabolic inhibitors. In some embodiments, the polymer decreases the level of at least one immune suppressive cell, Th2, Treg, Foxp3, Foxp3+ cell, GATA3, or GATA3+ cell in the presence of one or more metabolic inhibitors. Examples of metabolic inhibitors include, but are not limited to: glycolysis inhibitors, pentose phosphate pathway inhibitor, oxidative phosphorylation (OXPHOS) inhibitor, electron transport chain (ETC) inhibitor, TCA cycle inhibitors, polyamine pathway inhibitors, glutaminase inhibitors, CB-839, PFK15, inhibitors of hexokinase, inhibitors of phosphofructokinase, inhibitors of pyruvate kinase, inhibitors of lactate dehydrogenase, inhibitors of fatty acid oxidation (e.g., CPTla) and fatty acid synthase, inhibitors of enzymes involved in TCA cycle and/or polyamine pathway, or any combination thereof.

In various embodiments, the polymer is analyzed using a flow cytometry, enzyme linked immunosorbent assay (ELISA), immunohistochemistry (IHC), immunofluorescence (IF), or any combination thereof. Thus, in various embodiments, the function of the polymer is determined using a flow cytometry, ELISA, IHC, IF, or any combination thereof.

In one embodiment, the metabolite is a carbon-center metabolite. In one embodiment, the metabolite or derivative thereof modulates the function of an immune cell. Examples of carbon-center metabolites include, but are not limited to: phosphoenolpyruvate, phosphoenolpyruvic acid, glucono-lactone-6-phosphate, gluconate-6-phosphase, sedoheptulose-7-phosphate, ribulose, ribulose-5-phosphate(R5P), xylulose, xylulose-5-phosphate, fructose-1,6-biphosphate (F16BP), fructose 6 phosphate (F6P), fructose-2,6-biphosphate, glycerate-2-phosphate, glucerate-3-phosphate, malate, fumarate, succinate, isocitrate, citrate, cis-aconitate, malonyl-CoA, acetyl CoA, 3-methylbutyryl CoA, 2-methylbutyryl CoA, 3-ketoacyl CoA, 3-hydroxyacyl CoA, enoyl CoA, 3-ketoacyl functionalized metabolite, 3-hydroxyacyl functionalized metabolite, enoyl functionalized metabolite, fatty acids (e.g., caprylic acid, capric acid, lauric acid, myristic acid, palmitic acid, stearic acid, arachidic acid, behenic acid, lignoceric acid, cerotic acid, myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid, docosahexaenoic acid), or any combination thereof.

For example, in some embodiments, the metabolite is selected from fructose-1,6-biphosphate (F16BP), fructose 6 phosphate (F6P), ribulose 5 phosphate (R5P), phosphoenolpyruvic acid, or any combination thereof.

In one aspect, the invention provides a polymer compound or salt thereof comprising the structure of Formula (I)

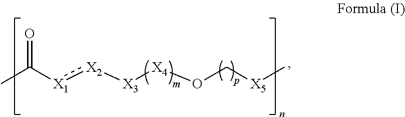

or a derivative or a pharmaceutical acceptable salt thereof.

Thus, in some aspects, the present invention provides a particle comprising a) a compound comprising the structure of Formula (I), or a derivative or a pharmaceutical acceptable salt thereof and b) an antigen or a fragment or variant thereof and/or a nucleic acid molecule or a fragment or variant thereof comprising a nucleotide sequence that encodes at least one antigen or a fragment or variant thereof.

In one embodiment, each occurrence of X₁ is independently C═R₁, CR₂, or CR₃R₄. In one embodiment, each occurrence of X₂ is independently C═R₁, CR₂, or CR₃R₄. In one embodiment, each occurrence of X₃ is independently C═R₁ or CR₃R₄. In one embodiment, each occurrence of X₄ is independently C═R₁ or CR₃R₄. In one embodiment, each occurrence of X₅ is independently O, S, C═R₁, CR₃R₄, NR₂, PR₂, or P(═R₁)(R₂).

In some embodiments, the bond between X₁ and X₂ is a single bond or a double bond. In one embodiment, when the bond between X₁ and X₂ is a single bond, X₁ and X₂ are each independently C═R₁ or CR₃R₄. In one embodiment, when the bond between X₁ and X₂ is a double bond, X₁ and X₂ are each C—R₂.

In one embodiment, each occurrence of R₁ is independently O, NH, or S. In one embodiment R₁ is O.

In one embodiment, each occurrence of R₂ is independently hydrogen, hydroxyl, carboxyl, alkyl, cycloalkyl, heterocycloalkyl, aryl, or heteroaryl. In one embodiment, R₂ is hydrogen. In one embodiment, R₂ is hydroxyl.

In one embodiment, each occurrence of R₃ is independently hydrogen, hydroxyl, carboxyl, alkyl, cycloalkyl, heterocycloalkyl, aryl, or heteroaryl. In one embodiment, P3 is hydrogen. In one embodiment, R₃ is hydroxyl. In one embodiment, R₃ is carboxyl.

In one embodiment, each occurrence of R₄ is independently hydrogen, hydroxyl, carboxyl, alkyl, cycloalkyl, heterocycloalkyl, aryl, or heteroaryl. In one embodiment, R₄ is hydrogen. In one embodiment, R₄ is hydroxyl. In one embodiment, R₄ is carboxyl.

In one embodiment, each occurrence of m is independently an integer represented by 0, 1, 2, or 5.

In one embodiment, each occurrence of p is independently an integer from 1 to 50. In one embodiment, each occurrence of p is independently an integer from 1 to 15. In one embodiment, each occurrence of p is independently an integer from 1 to 10. In one embodiment, p is 9. In one embodiment, p is 10.

In one embodiment, n is an integer from 1 to 1000.

In various embodiments, the polymer compound or salt thereof comprising the structure of Formula (I) is a compound comprising the structure of Formula (II)

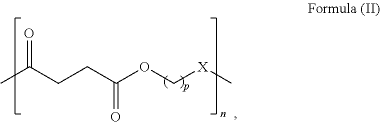

or a derivative or a pharmaceutical acceptable salt thereof.

In one embodiment, each occurrence of X is independently O, S, C═R₁, CR₃R₄, NR₂, PR₂, or P(═R₁)(R₂). In one embodiment, each occurrence of X is O. Thus, in various embodiments, the polymer compound or salt thereof comprising the structure of Formula (II) is a compound comprising the structure of Formula (III)

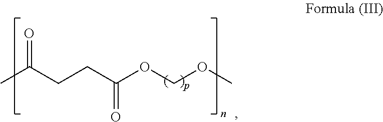

or a derivative or a pharmaceutical acceptable salt thereof.

In one embodiment, each occurrence of p is independently an integer from 1 to 50. In one embodiment, each occurrence of p is independently an integer from 1 to 15. In one embodiment, each occurrence of p is independently an integer from 1 to 10. In one embodiment, each occurrence of p is an integer represented by 2. In another embodiment, each occurrence of p is an integer represented by 9. In another embodiment, each occurrence of p is an integer represented by 10. Thus, in one embodiment, the compound comprising the structure of Formula (III) is polyethylenesuccinate (PEGS). In another embodiment, the compound comprising the structure of Formula (III) is polydecanesuccinate (PSA).

In one embodiment, n is an integer from 1 to 1000.

In various aspects, the particle comprises at least one nucleic acid molecule or a fragment or variant thereof comprising a nucleotide sequence that encodes at least one antigen or a fragment or variant thereof.

In some embodiments, the nucleic acid molecule comprises a nucleic acid molecule derived from a cancer cell, a synthetic nucleic acid molecule, or a combination thereof.

In one embodiment, the nucleic acid molecule is obtained from a sample. In one embodiment, the sample is a biopsy sample. In one embodiment, the sample is a cancer tissue sample. In one embodiment, the sample is a tumor tissue sample. In various embodiments, the nucleic acid molecule is obtained from a sample using any method known in the art.

In some embodiments, the nucleic acid molecule comprises a nucleic acid molecule or a fragment or variant thereof derived from a cancer cell.

In one embodiment, the cancer cell is a mammal cancer cell. In some embodiments, the cancer cell is a human cancer cell or canine cancer cell. For example, in one embodiment, the cancer cell is a melanoma cell. In one embodiment, the melanoma cell is a B16F10 cell, YUMM cell, and/or 4T1 cell. For example, in some embodiments, the nucleic acid molecule comprises an RNA or a fragment or variant thereof derived from a cancer cell. In some embodiments, the nucleic acid molecule comprises an isolated mRNA or a fragment or variant thereof derived from a B16F10 cell, YUMM cell, and/or 4T1 cell.

In one embodiment, the nucleic acid molecule is transcribed in vitro.

In one embodiment, the nucleic acid molecule is a synthetic nucleic acid molecule. Such synthetic nucleic acid molecule can be prepared using any method known in the art.

In one embodiment, the nucleic acid molecule is a modified nucleic acid molecule. Such modified nucleic acid molecule can be prepared using any method known in the art. For example, in some embodiments, modified nucleic acid molecule comprises a molecule in which one or more of the components of the nucleic acid, namely sugars, bases, and phosphate moieties, are different from those which occur in nature.

In some embodiments, the nucleic acid molecule or a fragment or variant thereof comprises a nucleotide sequence encoding at least one antigen or a fragment or variant thereof.

In some embodiments, the at least one antigen is a cancer antigen.

In some embodiments, the at least one antigen is any antigen described herein.

Examples of such antigens include, but are not limited to: an isolated protein or fragment thereof, isolated peptide or fragment thereof, tyrosinase-related protein or fragment thereof, tyrosinase-related protein 1 (TRP1) or fragment thereof, tyrosinase-related protein 2 (TRP2) or fragment thereof (e.g., TRP2 peptide comprising an amino acid sequence at least 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 1 or TRP2 peptide comprising the amino acid sequence as set forth in SEQ ID NO: 1), phosphorylated tyrosinase-related protein or fragment thereof, phosphorylated TRP1 or fragment thereof, phosphorylated TRP2 or fragment thereof (e.g., phosphorylated TRP2 (pTRP2) peptide comprising a phosphorylated amino acid sequence at least 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 1 or pTRP2 peptide comprising a phosphorylated amino acid sequence as set forth in SEQ ID NO: 1), melanocyte lineage/differentiation antigens, tyrosinase, human homologue of the mouse albino locus, glycoprotein 75 (gp 75), human homologue of the mouse brown locus, glycoprotein 100 (gp100), Pmel17, target for monoclonal antibody HMB45, human homologue of the mouse silver locus, Melan A/MART-1, oncofetal/cancer-testis antigens, melanoma antigen gene (MAGE) family proteins, B melanoma antigen (BAGE) peptides family, GAGE family antigens, esophageal squamous cell carcinoma-1 (NY-ESO-1), cancer-testis antigen 1B (CTAG1B), tumor-specific antigens, peptides with subtle mutations of normal cellular proteins (e.g., coding region mutations), cyclin-dependent kinase 4 or cell division protein kinase 4 (CDK4), β-catenin, mutated peptides activated as a result of cellular transformation, mutated introns, N-acetylglucosaminyltransferase V gene product, MUM-1, p15, antigens identified by monoclonal antibodies, gangliosides (e.g., GM2, GD2, GM3, and GD3), high molecular weight chondroitin sulfate proteoglycan, p97 melanotransferrin, SEREX antigens, D-1, synovial sarcoma/X breakpoint 2 (SSX-2), ovarian cancer antigens, surviving or baculoviral inhibitor of apoptosis repeat-containing 5 (BIRC5), cancer antigen 125 (CA125), carcinoembryonic antigen (CEA), DEAD-box helicase 43 (DDX43), epithelial cell adhesion molecule (EPCAM), folate Receptor Alpha (FOLR1), human epidermal growth factor receptor 2 (Her-2)/neu, melanoma-associated antigen 1 (MAGE-A1), melanoma-associated antigen 2 (MAGE-A2), melanoma-associated antigen 3 (MAGE-A3), melanoma-associated antigen 4 (MAGE-A4), melanoma-associated antigen 6 (MAGE-A6), melanoma-associated antigen 10 (MAGE-A10), melanoma-associated antigen 12 (MAGE-A12), mucin 1 (MUC-1), preferentially expressed antigen in melanoma (PRAME), tumor protein p53 (p53), trophoblast glycoprotein (TPBG), TRT, Wilms tumor protein (WT1), cancer/testis antigen 45 (CT45), breast cancer antigens, telomerase reverse transcriptase (hTERT), Sialyn-Tn, Wilms' Tumor Gene, antigens associated with cancers (e.g., acute lymphoblastic leukemia (ALL), acute myeloid leukemia (AML), cancer in adolescents, adrenocortical carcinoma, acquired immunodeficiency syndrome (AIDS)-related cancers, kaposi sarcoma, lymphoma, AIDS-related lymphoma, primary central nervous system (CNS) lymphoma, anal cancer, appendix cancer, gastrointestinal carcinoid tumors, astrocytomas, childhood astrocytomas, brain cancer, atypical teratoid/rhabdoid tumor, childhood atypical teratoid/rhabdoid tumor, CNS atypical teratoid/rhabdoid tumor, basal cell carcinoma of the skin, skin cancer, bile duct cancer, bladder cancer, childhood bladder cancer, bone cancer (includes Ewing sarcoma and osteosarcoma and malignant fibrous histiocytoma), brain tumors, breast cancer, bronchial tumors, Burkitt lymphoma, non-Hodgkin lymphoma, carcinoid tumor (gastrointestinal), childhood carcinoid tumors, carcinoma of unknown primary, childhood carcinoma of unknown primary, cardiac (heart) tumors, childhood cardiac (heart) tumors, medulloblastoma and other CNS embryonal tumors, childhood brain cancer, germ cell tumor, primary CNS lymphoma, cervical cancer, childhood cervical cancer, childhood cancers, unusual cancers of childhood, cholangiocarcinoma, chordoma, childhood chordoma, chronic lymphocytic leukemia (CLL), chronic myelogenous leukemia (CML), chronic myeloproliferative neoplasms, colorectal cancer, craniopharyngioma, childhood craniopharyngioma, mycosis fungoides and Sezary syndrome, ductal carcinoma in situ (DCIS), embryonal tumors, medulloblastoma and other childhood CNS brain cancers, endometrial cancer, ependymoma, childhood ependymoma, esophageal cancer, esthesioneuroblastoma, Ewing sarcoma, extracranial germ cell tumor, childhood extracranial germ cell tumor, extragonadal germ cell tumor, eye cancer, retinoblastoma, fallopian tube cancer, fibrous histiocytoma of bone, malignant fibrous histiocytoma of bone, osteosarcoma fibrous histiocytoma of bone, gallbladder cancer, gastric (stomach) cancer, gastrointestinal carcinoid tumor, gastrointestinal stromal tumors (GIST), soft tissue sarcoma, germ cell tumors, childhood CNS germ cell tumors, ovarian germ cell tumors, gestational trophoblastic disease, hairy cell leukemia, head and neck cancer, heart tumors, childhood heart tumors, hepatocellular (liver) cancer, histiocytosis, langerhans cell histiocytosis, Hodgkin lymphoma, hypopharyngeal cancer, Islet cell tumors, pancreatic neuroendocrine tumors, kidney (renal cell) cancer, Langerhans cell histiocytosis, Laryngeal cancer, leukemia, lip and oral cavity cancer, liver cancer, lung cancer, such as non-small cell, small cell, pleuropulmonary blastoma, and tracheobronchial tumor, male breast cancer, melanoma, childhood melanoma, intraocular (eye) melanoma, childhood intraocular melanoma, Merkel cell carcinoma, mesothelioma, malignant mesothelioma, metastatic cancer, metastatic squamous neck cancer with occult primary, midline tract carcinoma with NUT gene changes, mouth cancer, multiple endocrine neoplasia syndromes, multiple myeloma/plasma cell neoplasms, mycosis fungoides, myelodysplastic syndromes, myelodysplastic/myeloproliferative neoplasms, myelogenous leukemia, chronic myeloproliferative neoplasms, nasal cavity and paranasal sinus cancer, nasopharyngeal cancer, neuroblastoma, non-small cell lung cancer, oral cancer, lip and oral cavity cancer and oropharyngeal cancer, oropharyngeal cancer, ovarian cancer, childhood ovarian cancer, pancreatic cancer, pancreatic neuroendocrine tumors, papillomatosis, childhood laryngeal, paraganglioma, childhood paraganglioma, paranasal sinus and nasal cavity cancer, parathyroid cancer, penile cancer, pharyngeal cancer, pheochromocytoma, childhood pheochromocytoma, pituitary tumor, plasma cell neoplasm/multiple myeloma, pleuropulmonary blastoma, pregnancy and breast cancer, primary CNS lymphoma, primary peritoneal cancer, prostate cancer, rectal cancer, recurrent cancer, rhabdomyosarcoma, childhood rhabdomyosarcoma, childhood soft tissue sarcoma, salivary gland cancer, sarcoma, childhood vascular tumors, osteosarcoma, uterine sarcoma, Sezary syndrome, childhood skin cancer, small cell lung cancer, small intestine cancer, squamous cell carcinoma of the skin, squamous neck cancer with occult primary, metastatic squamous neck cancer with occult primary, stomach (gastric) cancer, T-cell lymphoma, cutaneous T-cell lymphoma, testicular cancer, childhood testicular cancer, throat cancer, nasopharyngeal cancer, thymoma and thymic carcinoma, thyroid cancer, tracheobronchial tumors, transitional cell cancer of the renal pelvis and ureter, urethral cancer, uterine cancer, endometrial uterine cancer, vaginal cancer, childhood vaginal cancer, vascular tumors, vulvar cancer, Wilms tumor and other childhood kidney tumors, and cancers in young adults), or any combination thereof.

In one embodiment, at least part of the nucleic acid molecule is encapsulated by the polymer. In one embodiment, the nucleic acid molecule is encapsulated by the polymer.

In one embodiment, the nucleic acid molecule is operably linked to the metabolite or derivative or polymer thereof. In one embodiment, the nucleic acid molecule is operably linked to the compound comprising the structure of Formula (I), or a derivative or a pharmaceutical acceptable salt thereof. In one embodiment, the nucleic acid molecule is operably linked to the compound comprising the structure of Formula (II), or a derivative or a pharmaceutical acceptable salt thereof. In one embodiment, the nucleic acid molecule is operably linked to the compound comprising the structure of Formula (III), or a derivative or a pharmaceutical acceptable salt thereof.

In one embodiment, the nucleic acid molecule is an RNA, mRNA, tRNA, rRNA, DNA, or any combination thereof.

For example, in some embodiments, the particle comprises an RNA. In one embodiment, the RNA is isolated from a sample. In one embodiment, the RNA is transcribed in vitro.

In one embodiment, the RNA is a synthetic RNA. Such synthetic RNA can be prepared using any method known in the art.

In one embodiment, the RNA is a modified RNA. Such modified RNA can be prepared using any method known in the art. For example, in some embodiments, modified RNA comprises a molecule in which one or more of the components of the nucleic acid, namely sugars, bases, and phosphate moieties, are different from those which occur in nature.

In some embodiments, the RNA comprises an RNA or a fragment or variant thereof derived from a cancer cell.

In one embodiment, the cancer cell is a mammal cancer cell. In some embodiments, the cancer cell is a human cancer cell or canine cancer cell. For example, in one embodiment, the cancer cell is a melanoma cell. In one embodiment, the melanoma cell is a B16F10 cell and/or YUMM cell.

In one embodiment, at least part of the RNA is encapsulated by the polymer. In one embodiment, the RNA is encapsulated by the polymer.

In one embodiment, the RNA is operably linked to the metabolite or derivative thereof. In one embodiment, the RNA is operably linked to the compound comprising the structure of Formula (I), or a derivative or a pharmaceutical acceptable salt thereof. In one embodiment, the RNA is operably linked to the compound comprising the structure of Formula (II), or a derivative or a pharmaceutical acceptable salt thereof. In one embodiment, the RNA is operably linked to the compound comprising the structure of Formula (III), or a derivative or a pharmaceutical acceptable salt thereof.

In various aspects, the particle comprises an antigen or a fragment or variant thereof. In some embodiments, the antigen comprises an amino acid sequence. In various embodiments, the amino acid sequence comprises one or more amino acids. In one embodiment, the amino acid is a phosphorylated amino acid.

In some embodiments, the amino acid sequence is a peptide or fragment thereof, protein or fragment thereof, or any combination thereof. In various embodiments, the amino acid sequence is a phosphorylated amino acid sequence. Thus, in some embodiments, the amino acid sequence is a phosphorylated peptide or phosphorylated fragment thereof, phosphorylated protein or phosphorylated fragment thereof, or any combination thereof.

Examples of amino acid sequences include, but are not limited to: an isolated protein or fragment thereof, isolated peptide or fragment thereof, tyrosinase-related protein or fragment thereof, tyrosinase-related protein 1 (TRP1) or fragment thereof, tyrosinase-related protein 2 (TRP2) or fragment thereof (e.g., TRP2 peptide comprising an amino acid sequence at least 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 1 or TRP2 peptide comprising the amino acid sequence as set forth in SEQ ID NO: 1), phosphorylated tyrosinase-related protein or fragment thereof, phosphorylated TRP1 or fragment thereof, phosphorylated TRP2 or fragment thereof (e.g., phosphorylated TRP2 (pTRP2) peptide comprising a phosphorylated amino acid sequence at least 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 1 or pTRP2 peptide comprising a phosphorylated amino acid sequence as set forth in SEQ ID NO: 1), melanocyte lineage/differentiation antigens, tyrosinase, human homologue of the mouse albino locus, glycoprotein 75 (gp 75), human homologue of the mouse brown locus, glycoprotein 100 (gp100), Pmel17, target for monoclonal antibody HMB45, human homologue of the mouse silver locus, Melan A/MART-1, oncofetal/cancer-testis antigens, melanoma antigen gene (MAGE) family proteins, B melanoma antigen (BAGE) peptides family, GAGE family antigens, esophageal squamous cell carcinoma-1 (NY-ESO-1), cancer-testis antigen 1B (CTAGIB), tumor-specific antigens, peptides with subtle mutations of normal cellular proteins (e.g., coding region mutations), cyclin-dependent kinase 4 or cell division protein kinase 4 (CDK4), β-catenin, mutated peptides activated as a result of cellular transformation, mutated introns, N-acetylglucosaminyltransferase V gene product, MUM-1, p15, antigens identified by monoclonal antibodies, gangliosides (e.g., GM2, GD2, GM3, and GD3), high molecular weight chondroitin sulfate proteoglycan, p97 melanotransferrin, SEREX antigens, D-1, synovial sarcoma/X breakpoint 2 (SSX-2), ovarian cancer antigens, surviving or baculoviral inhibitor of apoptosis repeat-containing 5 (BIRC5), cancer antigen 125 (CA125), carcinoembryonic antigen (CEA), DEAD-box helicase 43 (DDX43), epithelial cell adhesion molecule (EPCAM), folate Receptor Alpha (FOLR1), human epidermal growth factor receptor 2 (Her-2)/neu, melanoma-associated antigen 1 (MAGE-A1), melanoma-associated antigen 2 (MAGE-A2), melanoma-associated antigen 3 (MAGE-A3), melanoma-associated antigen 4 (MAGE-A4), melanoma-associated antigen 6 (MAGE-A6), melanoma-associated antigen 10 (MAGE-A10), melanoma-associated antigen 12 (MAGE-A12), mucin 1 (MUC-1), preferentially expressed antigen in melanoma (PRAME), tumor protein p53 (p53), trophoblast glycoprotein (TPBG), TRT, Wilms tumor protein (WT1), cancer/testis antigen 45 (CT45), breast cancer antigens, telomerase reverse transcriptase (hTERT), Sialyn-Tn, Wilms' Tumor Gene, antigens associated with cancers (e.g., acute lymphoblastic leukemia (ALL), acute myeloid leukemia (AML), cancer in adolescents, adrenocortical carcinoma, acquired immunodeficiency syndrome (AIDS)-related cancers, kaposi sarcoma, lymphoma, AIDS-related lymphoma, primary central nervous system (CNS) lymphoma, anal cancer, appendix cancer, gastrointestinal carcinoid tumors, astrocytomas, childhood astrocytomas, brain cancer, atypical teratoid/rhabdoid tumor, childhood atypical teratoid/rhabdoid tumor, CNS atypical teratoid/rhabdoid tumor, basal cell carcinoma of the skin, skin cancer, bile duct cancer, bladder cancer, childhood bladder cancer, bone cancer (includes Ewing sarcoma and osteosarcoma and malignant fibrous histiocytoma), brain tumors, breast cancer, bronchial tumors, Burkitt lymphoma, non-Hodgkin lymphoma, carcinoid tumor (gastrointestinal), childhood carcinoid tumors, carcinoma of unknown primary, childhood carcinoma of unknown primary, cardiac (heart) tumors, childhood cardiac (heart) tumors, medulloblastoma and other CNS embryonal tumors, childhood brain cancer, germ cell tumor, primary CNS lymphoma, cervical cancer, childhood cervical cancer, childhood cancers, unusual cancers of childhood, cholangiocarcinoma, chordoma, childhood chordoma, chronic lymphocytic leukemia (CLL), chronic myelogenous leukemia (CML), chronic myeloproliferative neoplasms, colorectal cancer, craniopharyngioma, childhood craniopharyngioma, mycosis fungoides and Sezary syndrome, ductal carcinoma in situ (DCIS), embryonal tumors, medulloblastoma and other childhood CNS brain cancers, endometrial cancer, ependymoma, childhood ependymoma, esophageal cancer, esthesioneuroblastoma, Ewing sarcoma, extracranial germ cell tumor, childhood extracranial germ cell tumor, extragonadal germ cell tumor, eye cancer, retinoblastoma, fallopian tube cancer, fibrous histiocytoma of bone, malignant fibrous histiocytoma of bone, osteosarcoma fibrous histiocytoma of bone, gallbladder cancer, gastric (stomach) cancer, gastrointestinal carcinoid tumor, gastrointestinal stromal tumors (GIST), soft tissue sarcoma, germ cell tumors, childhood CNS germ cell tumors, ovarian germ cell tumors, gestational trophoblastic disease, hairy cell leukemia, head and neck cancer, heart tumors, childhood heart tumors, hepatocellular (liver) cancer, histiocytosis, langerhans cell histiocytosis, Hodgkin lymphoma, hypopharyngeal cancer, Islet cell tumors, pancreatic neuroendocrine tumors, kidney (renal cell) cancer, Langerhans cell histiocytosis, Laryngeal cancer, leukemia, lip and oral cavity cancer, liver cancer, lung cancer, such as non-small cell, small cell, pleuropulmonary blastoma, and tracheobronchial tumor, male breast cancer, melanoma, childhood melanoma, intraocular (eye) melanoma, childhood intraocular melanoma, Merkel cell carcinoma, mesothelioma, malignant mesothelioma, metastatic cancer, metastatic squamous neck cancer with occult primary, midline tract carcinoma with NUT gene changes, mouth cancer, multiple endocrine neoplasia syndromes, multiple myeloma/plasma cell neoplasms, mycosis fungoides, myelodysplastic syndromes, myelodysplastic/myeloproliferative neoplasms, myelogenous leukemia, chronic myeloproliferative neoplasms, nasal cavity and paranasal sinus cancer, nasopharyngeal cancer, neuroblastoma, non-small cell lung cancer, oral cancer, lip and oral cavity cancer and oropharyngeal cancer, oropharyngeal cancer, ovarian cancer, childhood ovarian cancer, pancreatic cancer, pancreatic neuroendocrine tumors, papillomatosis, childhood laryngeal, paraganglioma, childhood paraganglioma, paranasal sinus and nasal cavity cancer, parathyroid cancer, penile cancer, pharyngeal cancer, pheochromocytoma, childhood pheochromocytoma, pituitary tumor, plasma cell neoplasm/multiple myeloma, pleuropulmonary blastoma, pregnancy and breast cancer, primary CNS lymphoma, primary peritoneal cancer, prostate cancer, rectal cancer, recurrent cancer, rhabdomyosarcoma, childhood rhabdomyosarcoma, childhood soft tissue sarcoma, salivary gland cancer, sarcoma, childhood vascular tumors, osteosarcoma, uterine sarcoma, Sezary syndrome, childhood skin cancer, small cell lung cancer, small intestine cancer, squamous cell carcinoma of the skin, squamous neck cancer with occult primary, metastatic squamous neck cancer with occult primary, stomach (gastric) cancer, T-cell lymphoma, cutaneous T-cell lymphoma, testicular cancer, childhood testicular cancer, throat cancer, nasopharyngeal cancer, thymoma and thymic carcinoma, thyroid cancer, tracheobronchial tumors, transitional cell cancer of the renal pelvis and ureter, urethral cancer, uterine cancer, endometrial uterine cancer, vaginal cancer, childhood vaginal cancer, vascular tumors, vulvar cancer, Wilms tumor and other childhood kidney tumors, and cancers in young adults), or any combination thereof.

In one embodiment, at least part of the amino acid sequence is encapsulated by the polymer. In one embodiment, the amino acid sequence is encapsulated by the polymer.

In one embodiment, the amino acid sequence is operably linked to the metabolite or derivative or polymer thereof. In one embodiment, the amino acid sequence is operably linked to the compound comprising the structure of Formula (I), or a derivative or a pharmaceutical acceptable salt thereof. In one embodiment, the amino acid sequence is operably linked to the compound comprising the structure of Formula (II), or a derivative or a pharmaceutical acceptable salt thereof. In one embodiment, the amino acid sequence is operably linked to the compound comprising the structure of Formula (III), or a derivative or a pharmaceutical acceptable salt thereof.

In some embodiments, the particle further comprises an adjuvant. Examples of such adjuvants include, but are not limited to: polyinosinic:polycytidylic adic (poly(I:C)) or analog thereof, muramyl dipeptide derivatives (MDP) or analog thereof, Alum and Emulsions, complete Freund's adjuvant (CFA), incomplete Freund's adjuvant (IFA), pattern recognition receptor (PRR) ligands, cyclic guanosine monophosphate-adenosine monophosphate (2′3′-cGAMP), bis-(3′-5′)-cyclic dimeric adenosine monophosphate (c-di-AMP), Rp,Rp-isomer of the 2′3′-bisphosphorothioate analog of 3′3′-cyclic adenosine monophosphate (2′3′-c-di-AM(PS)2 (Rp,Rp)), cyclic diguanylate monophosphate-stimulator of interferon genes (c-di-GMP STING)-based vaccine adjuvant, CL401, CL413, CL429, Flagellin, Imiquimod, lipopolysaccharide (LPS) from the gram-negative bacteria E. coli 0111:B4 (LPS-EB), monophosphoryl lipid A from Salmonella minnesota R595 lipopolysaccharide (MPLA-SM), synthetic monophosphoryl lipid A (MPLA), oligodeoxynucleotides (ODN) 1585, ODN 1826, ODN 2006, ODN 2395, Pam3CSK4, Resiquimod (R848), trehalose-6,6-dibehenate (TDB), or any combination thereof. For example, in some embodiments, the adjuvant is polyinosinic:polycytidylic acid (poly(I.C)).

In one embodiment, at least part of the adjuvant is encapsulated by the polymer. In one embodiment, the adjuvant is encapsulated by the polymer.

In one embodiment, the adjuvant is operably linked to the metabolite or derivative thereof. In one embodiment, the adjuvant is operably linked to the nucleic acid molecule and/or amino acid sequence. In one embodiment, the adjuvant is operably linked to the metabolite or derivative thereof and the nucleic acid molecule and/or amino acid sequence. In one embodiment, the adjuvant is operably linked to the compound comprising the structure of Formula (I), or a derivative or a pharmaceutical acceptable salt thereof, the nucleic acid molecule and/or amino acid sequence, or both. In one embodiment, the adjuvant is operably linked to the compound comprising the structure of Formula (II), or a derivative or a pharmaceutical acceptable salt thereof, the nucleic acid molecule and/or amino acid sequence, or both. In one embodiment, the adjuvant is operably linked to the compound comprising the structure of Formula (III), or a derivative or a pharmaceutical acceptable salt thereof, the nucleic acid molecule and/or amino acid sequence, or both.

Thus, in one aspect, the invention provides a polymer compound or salt thereof comprising at least one nucleic acid molecule, at least one metabolite, at least one adjuvant, at least one metal, or any combination thereof. In one embodiment, the invention provides a polymer compound or salt thereof comprising at least one nucleic acid molecule, at least one metabolite, at least one adjuvant, at least one metal, or any combination thereof that are operably linked to each other in any order. For example, in one aspect, the invention provides a polymer compound or salt thereof comprising the structure of

or a derivative or a pharmaceutical acceptable salt thereof,
or a derivative or a pharmaceutical acceptable salt thereof,
or a derivative or a pharmaceutical acceptable salt thereof, or
or a derivative or a pharmaceutical acceptable salt thereof.

In another aspect, the invention provides a polymer compound or salt thereof comprising at least one amino acid sequence, at least one metabolite, at least one adjuvant, at least one metal, or any combination thereof. In one embodiment, the invention provides a polymer compound or salt thereof comprising at least one amino acid sequence, at least one metabolite, at least one adjuvant, at least one metal, or any combination thereof that are operably linked to each other in any order. For example, in one aspect, the invention provides a polymer compound or salt thereof comprising the structure of

or a derivative or a pharmaceutical acceptable salt thereof,
or a derivative or a pharmaceutical acceptable salt thereof,
or a derivative or a pharmaceutical acceptable salt thereof, or
or a derivative or a pharmaceutical acceptable salt thereof.

In some embodiments, each occurrence of M is independently Ca, Mg, Na, K, Sr, Zn, Fe, Co, or Cu.

In some embodiments, each occurrence of metabolite is independently a metabolite or derivative thereof.

In some embodiments, each occurrence of n is independently an integer from 1 to 1000.

In some embodiments, each occurrence of p is independently an integer represented by 0 or 1.

In various embodiments, the polymer compound or salt thereof comprising the structure of Formula (VIII) is a compound comprising the structure of

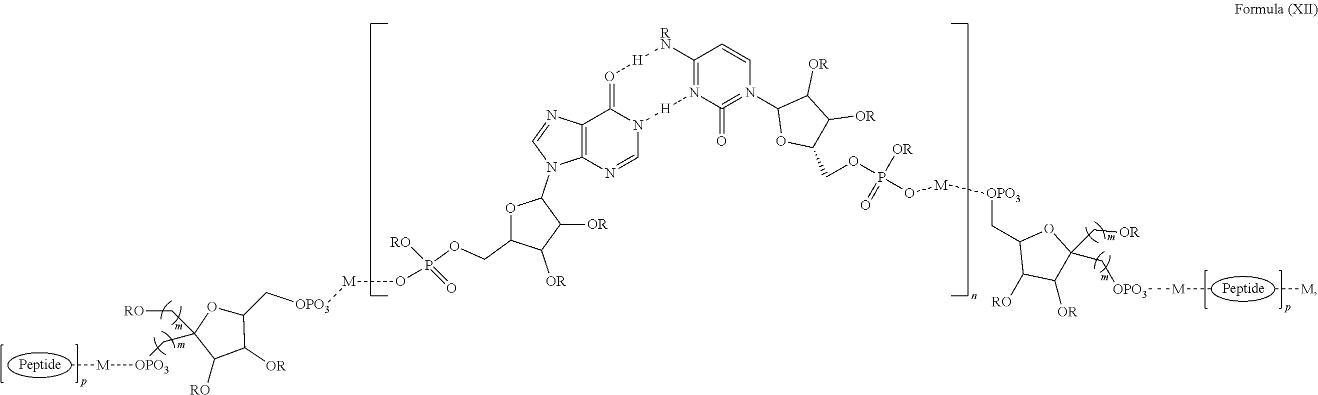

or a derivative or a pharmaceutical acceptable salt thereof.

In some embodiments, each occurrence of M is independently Ca, Mg, Na, K, Sr, Zn, Fe, Co, or Cu.

In some embodiments, each occurrence of R is independently hydrogen, hydroxyl, carboxyl, alkyl, cycloalkyl, heterocycloalkyl, aryl, or heteroaryl.

In some embodiments, each occurrence of m is independently an integer represented by 0 or 1.

In some embodiments, each occurrence of n is independently an integer from 1 to 1000.

In some embodiments, each occurrence of p is independently an integer represented by 0 or 1.

In one embodiment, the peptide is phosphorylated TRP2 peptide, each occurrence of M is Ca, each occurrence of R is hydrogen, and each occurrence of p is an integer represented by 1. Thus, in one embodiment, the compound comprising the structure of Formula (VIII) is a compound comprising the structure of

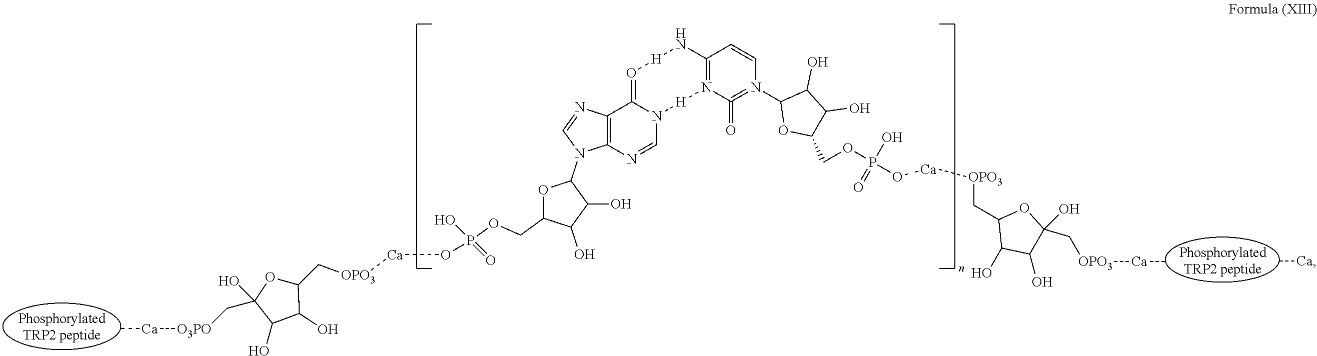

or a derivative or a pharmaceutical acceptable salt thereof. For example, in some embodiments, the phosphorylated TRP2 peptide comprises a phosphorylated amino acid sequence at least 50%, 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 1. In one embodiment, the phosphorylated TRP2 peptide comprises a phosphorylated amino acid sequence as set forth in SEQ ID NO: 1).

In some embodiments, the polymer is phagocytosed by a cell. Examples of such cells include, but are not limited to, antigen-presenting cells (APC), accessory cells, dendritic cells, T cells, B cells, macrophages, natural killer cells, neutrophils, eosinophils, basophils, and/or any other immune cells.

In one aspect, the present invention also provides a particle comprising at least one polymer described herein. In one embodiment, the particle is a microparticle. In one embodiment, the particle is a nanoparticle. For example, in one embodiment, one or more compounds comprising the structure of Formula (I), or a derivative or a pharmaceutical acceptable salt thereof, form the nanoparticle. Thus, in various embodiments, the present invention discloses a nanoparticle comprising at least one compound or salt thereof comprising the structure of Formula (I).

In another embodiment, one or more compounds comprising the structure of Formula (II), or a derivative or a pharmaceutical acceptable salt thereof, form the nanoparticle. Thus, in various embodiments, the present invention discloses a nanoparticle comprising at least one compound or salt thereof comprising the structure of Formula (II).

In another embodiment, one or more compounds comprising the structure of Formula (III), or a derivative or a pharmaceutical acceptable salt thereof, form the nanoparticle. Thus, in various embodiments, the present invention discloses a nanoparticle comprising at least one compound or salt thereof comprising the structure of Formula (III).

In another embodiment, one or more compounds comprising the structure of Formula (IV), or a derivative or a pharmaceutical acceptable salt thereof, form the nanoparticle. Thus, in various embodiments, the present invention discloses a nanoparticle comprising at least one compound or salt thereof comprising the structure of Formula (IV).

In another embodiment, one or more compounds comprising the structure of Formula (V), or a derivative or a pharmaceutical acceptable salt thereof, form the nanoparticle. Thus, in various embodiments, the present invention discloses a nanoparticle comprising at least one compound or salt thereof comprising the structure of Formula (V).

In another embodiment, one or more compounds comprising the structure of Formula (VI), or a derivative or a pharmaceutical acceptable salt thereof, form the nanoparticle. Thus, in various embodiments, the present invention discloses a nanoparticle comprising at least one compound or salt thereof comprising the structure of Formula (VI).

In another embodiment, one or more compounds comprising the structure of Formula (VII), or a derivative or a pharmaceutical acceptable salt thereof, form the nanoparticle. Thus, in various embodiments, the present invention discloses a nanoparticle comprising at least one compound or salt thereof comprising the structure of Formula (VII).

In another embodiment, one or more compounds comprising the structure of Formula (VIII), or a derivative or a pharmaceutical acceptable salt thereof, form the nanoparticle. Thus, in various embodiments, the present invention discloses a nanoparticle comprising at least one compound or salt thereof comprising the structure of Formula (VIII).

In another embodiment, one or more compounds comprising the structure of Formula (IX), or a derivative or a pharmaceutical acceptable salt thereof, form the nanoparticle. Thus, in various embodiments, the present invention discloses a nanoparticle comprising at least one compound or salt thereof comprising the structure of Formula (IX).

In another embodiment, one or more compounds comprising the structure of Formula (X), or a derivative or a pharmaceutical acceptable salt thereof, form the nanoparticle. Thus, in various embodiments, the present invention discloses a nanoparticle comprising at least one compound or salt thereof comprising the structure of Formula (X).

In another embodiment, one or more compounds comprising the structure of Formula (XI), or a derivative or a pharmaceutical acceptable salt thereof, form the nanoparticle. Thus, in various embodiments, the present invention discloses a nanoparticle comprising at least one compound or salt thereof comprising the structure of Formula (XI).

In another embodiment, one or more compounds comprising the structure of Formula (XII), or a derivative or a pharmaceutical acceptable salt thereof, form the nanoparticle. Thus, in various embodiments, the present invention discloses a nanoparticle comprising at least one compound or salt thereof comprising the structure of Formula (XII).

In another embodiment, one or more compounds comprising the structure of Formula (XIII), or a derivative or a pharmaceutical acceptable salt thereof, form the nanoparticle. Thus, in various embodiments, the present invention discloses a nanoparticle comprising at least one compound or salt thereof comprising the structure of Formula (XIII).

In some embodiments, the nanoparticle has an average size (i.e., average diameter of the nanoparticle) of about 0.01 nm to about 10000 nm. For example, in one embodiment, the nanoparticle has an average size (i.e., average diameter of the nanoparticle) of about 0.01 nm. In another embodiment, the nanoparticle has an average size (i.e., average diameter of the nanoparticle) of about 10 nm.

In some embodiments, the nanoparticle is a microparticle. In some embodiments, the microparticle has an average size (i.e., average diameter of the microparticle) of about 0.01 μm to about 1000 μm. For example, in one embodiment, the microparticle has an average size (i.e., average diameter of the microparticle) of about 0.01 μm. In another embodiment, the microparticle has an average size (i.e., average diameter of the microparticle) of about 10 μm. In some embodiments, the particle has a diameter of between about 1 μm to about 100 μm. In one embodiment, the particle has a diameter of about 2 μm.

In some embodiments, the nanoparticle is any type of nanoparticle, including, but not limited to, a nanoparticle cluster, nanovesicle, nanocarrier, microcapsule, ectosomes, micellar nanoparticles, lamellae shaped nanoparticles, polymersome nanoparticles, polymer vesicle, and micro-size particles of various other small fabrications that are known to those in the art.

In some embodiments, the nanoparticle is a biodegradable nanoparticle. For example, in one embodiment, the nanoparticle is biodegradable nanocapsule. In another embodiment, the nanoparticle is a biodegradable polymer vesicle.

In some embodiments, the nanoparticle is phagocytosed by a cell (e.g., immune cell).

In various embodiments, the nanoparticle is analyzed using a flow cytometry, ELISA, IHC, IF, or any combination thereof. Thus, in various embodiments, the function of the nanoparticle is determined using a flow cytometry, ELISA, IHC, IF, or any combination thereof.

In one embodiment, the nanoparticle modulates the function of a cell (e.g., immune cell). In one embodiment, the nanoparticle prevents a metabolic inhibition in a cell (e.g., immune cell). In one embodiment, the nanoparticle treats a metabolic inhibition in a cell (e.g., immune cell). In one embodiment, the nanoparticle induces an activation of a cell (e.g., immune cell). In one embodiment, the nanoparticle induces glycolysis in a cell (e.g., immune cell). In one embodiment, the nanoparticle induces a TCA cycle in a cell (e.g., immune cell). In one embodiment, the nanoparticle induces a polyamine pathway in a cell (e.g., immune cell). In one embodiment, the nanoparticle induces a PPP in a cell (e.g., immune cell). In one embodiment, the nanoparticle induces an ECAR in a cell (e.g., immune cell). In one embodiment, the nanoparticle induces an OCR in a cell (e.g., immune cell). In one embodiment, the nanoparticle induces a mitochondrial respiration in a cell (e.g., immune cell). In one embodiment, the nanoparticle induces a release of a metabolite in a cell (e.g., immune cell). In one embodiment, the nanoparticle induces a pro-inflammatory response in a cell (e.g., immune cell). In one embodiment, the nanoparticle induces one or more BRAF inhibitors in a cell (e.g., immune cell). In one embodiment, the nanoparticle induces one or more tyrosine kinase inhibitors in a cell (e.g., immune cell). In one embodiment, the nanoparticle induces one or more ERK inhibitors in a cell (e.g., immune cell). In one embodiment, the nanoparticle induces one or more MEK inhibitors in a cell (e.g., immune cell). In one embodiment, the nanoparticle induces a cancer cell suppression. In one embodiment, the nanoparticle reduces a cancer cell proliferation. In some embodiments, the nanoparticle increases the level of at least one T cell, Tc1, Tc2, Tc17, Th, Th1, Th17, or Teff in a cell. In some embodiments, the nanoparticle decreases the level of at least one immune suppressive cell, Th2, Treg, Foxp3+ cell, or GATA3+ cell. In some embodiments, the nanoparticle decreases the level of at least one Foxp3 or GATA3 in a cell.

In one embodiment, the nanoparticle modulates the function of an immune cell in the presence of one or more metabolic inhibitors, metabolic pathway accelerators, peptide therapeutics, protein therapeutics, and/or enzymes to accelerate metabolism. For example, in one embodiment, the nanoparticle prevents a metabolic inhibition in a cell in the presence of one or more metabolic inhibitors. In one embodiment, the nanoparticle treats a metabolic inhibition in a cell in the presence of one or more metabolic inhibitors. In one embodiment, the nanoparticle induces an activation of a cell in the presence of one or more metabolic inhibitors. In one embodiment, the nanoparticle induces glycolysis in a cell in the presence of one or more metabolic inhibitors. In one embodiment, the nanoparticle induces a TCA cycle in a cell in the presence of one or more metabolic inhibitors. In one embodiment, the nanoparticle induces a polyamine pathway in a cell in the presence of one or more metabolic inhibitors. In one embodiment, the nanoparticle induces a PPP in a cell in the presence of one or more metabolic inhibitors. In one embodiment, the nanoparticle induces an ECAR in a cell in the presence of one or more metabolic inhibitors. In one embodiment, the nanoparticle induces an OCR in a cell in the presence of one or more metabolic inhibitors. In one embodiment, the nanoparticle induces a mitochondrial respiration in a cell in the presence of one or more metabolic inhibitors. In one embodiment, the nanoparticle induces a release of a metabolite in a cell in the presence of one or more metabolic inhibitors. In one embodiment, the nanoparticle induces a pro-inflammatory response in a cell in the presence of one or more metabolic inhibitors. In one embodiment, the nanoparticle induces one or more BRAF inhibitors in a cell in the presence of one or more metabolic inhibitors. In one embodiment, the nanoparticle induces one or more tyrosine kinase inhibitors in a cell in the presence of one or more metabolic inhibitors. In one embodiment, the nanoparticle induces one or more ERK inhibitors in a cell in the presence of one or more metabolic inhibitors. In one embodiment, the nanoparticle induces one or more MEK inhibitors in a cell in the presence of one or more metabolic inhibitors. In one embodiment, the nanoparticle induces a cancer cell suppression in the presence of one or more metabolic inhibitors. In one embodiment, the nanoparticle reduces a cancer cell proliferation in the presence of one or more metabolic inhibitors. In some embodiments, the nanoparticle increases the level of at least one T cell, Tc1, Tc2, Tc17, Th, Th1, Th17, or Teff in a cell in the presence of one or more metabolic inhibitors. In some embodiments, the nanoparticle decreases the level of at least one immune suppressive cell, Th2, Treg, Foxp3+ cell, or GATA3+ cell. In some embodiments, the nanoparticle decreases the level of at least one Foxp3 or GATA3 in a cell in the presence of one or more metabolic inhibitors.

Examples of such cells include, but are not limited to, immune cells, antigen-presenting cells (APC), accessory cells, dendritic cells, T cells, B cells, macrophages, natural killer cells, neutrophils, eosinophils, basophils, and/or any other immune cells.

In one aspect of the invention, the nanoparticle comprises at least one therapeutic agent. In one embodiment, the therapeutic agent is encapsulated by the nanoparticle. In one embodiment, the therapeutic agent is operably linked to the nanoparticle. In one embodiment, the therapeutic agent is encapsulated by the polymer. In one embodiment, the therapeutic agent is operably linked to the polymer.

In one embodiment, the nanoparticle releases at least one therapeutic agent. In one embodiment, the nanoparticle releases at least one therapeutic agent inside or outside the cell. In some embodiments, the nanoparticle decomposes or degrades to release at least one therapeutic agent. Examples of such therapeutic agents include, but are not limited to, one or more drugs, metabolites, metabolic inhibitors, metabolic pathway accelerators, peptide therapeutics, protein therapeutics, enzymes to accelerate metabolism, proteins, amino acids, peptides, antibodies, medical imaging agents, therapeutic moieties, one or more non-therapeutic moieties or a combination to target cancer or atherosclerosis, selected from folic acid, peptides, proteins, aptamers, antibodies, siRNA, poorly water soluble drugs, anti-cancer drugs, antibiotics, analgesics, vaccines, anticonvulsants; anti-diabetic agents, antifungal agents, antineoplastic agents, anti-parkinsonian agents, anti-rheumatic agents, appetite suppressants, biological response modifiers, cardiovascular agents, central nervous system stimulants, contraceptive agents, dietary supplements, vitamins, minerals, lipids, saccharides, metals, amino acids (and precursors), nucleic acids and precursors, contrast agents, diagnostic agents, dopamine receptor agonists, erectile dysfunction agents, fertility agents, gastrointestinal agents, hormones, immunomodulators, antihypercalcemia agents, mast cell stabilizers, muscle relaxants, nutritional agents, ophthalmic agents, osteoporosis agents, psychotherapeutic agents, parasympathomimetic agents, parasympatholytic agents, respiratory agents, sedative hypnotic agents, skin and mucous membrane agents, smoking cessation agents, steroids, sympatholytic agents, urinary tract agents, uterine relaxants, vaginal agents, vasodilator, anti-hypertensive, hyperthyroids, anti-hyperthyroids, anti-asthmatics and vertigo agents, or any combinations thereof.

The inhibitors of the invention can be administered alone or in combination with other anti-tumor agents, including cytotoxic/antineoplastic agents and anti-angiogenic agents. Cytotoxic/anti-neoplastic agents are defined as agents which attack and kill cancer cells. Some cytotoxic/anti-neoplastic agents are alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents are antimetabolites for tumor cells, e.g., cytosine arabinoside, fluorouracil, methotrexate, mercaptopuirine, azathioprime, and procarbazine. Other cytotoxic/anti-neoplastic agents are antibiotics, e.g., doxorubicin, bleomycin, dactinomycin, daunorubicin, mithramycin, mitomycin, mytomycin C, and daunomycin. There are numerous liposomal formulations commercially available for these compounds. Still other cytotoxic/anti-neoplastic agents are mitotic inhibitors (vinca alkaloids). These include vincristine, vinblastine and etoposide. Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine.

Anti-angiogenic agents are well known to those of skill in the art. Suitable anti-angiogenic agents for use in the methods and compositions of the present disclosure include anti-VEGF antibodies, including humanized and chimeric antibodies, anti-VEGF aptamers and antisense oligonucleotides. Other known inhibitors of angiogenesis include angiostatin, endostatin, interferons, interleukin 1 (including alpha and beta) interleukin 12, retinoic acid, and tissue inhibitors of metalloproteinase-1 and -2. (TIMP-1 and -2). Small molecules, including topoisomerases such as razoxane, a topoisomerase II inhibitor with anti-angiogenic activity, can also be used.

Other anti-cancer agents that can be used in combination with the disclosed compounds include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. In one embodiment, the anti-cancer drug is 5-fluorouracil, taxol, or leucovorin.

In some embodiments, the anti-cancer agent may be a prodrug form of an anti-cancer agent. As used herein, the term “prodrug form” and its derivatives is used to refer to a drug that has been chemically modified to add and/or remove one or more substituents in such a manner that, upon introduction of the prodrug form into a subject, such a modification may be reversed by naturally occurring processes, thus reproducing the drug. The use of a prodrug form of an anti-cancer agent in the compositions, among other things, may increase the concentration of the anti-cancer agent in the compositions of the present disclosure. In certain embodiments, an anti-cancer agent may be chemically modified with an alkyl or acyl group or some form of lipid. The selection of such a chemical modification, including the substituent(s) to add and/or remove to create the prodrug, may depend upon a number of factors including, but not limited to, the particular drug and the desired properties of the prodrug. One of ordinary skill in the art, with the benefit of this disclosure, will recognize suitable chemical modifications.

In one embodiment, the therapeutic agent is one or more non-therapeutic moieties. In some embodiments, the nanoparticle comprises one or more therapeutic moieties, one or more non-therapeutic moieties, or any combination thereof. In some embodiments, the composition comprises folic acid, peptides, proteins, aptamers, antibodies, small RNA molecules, miRNA, shRNA, siRNA, poorly water-soluble therapeutic agents, anti-cancer agents, or any combinations thereof.

In another aspect of the invention, the nanoparticle releases at least one metabolite. In some embodiments, the nanoparticle decomposes or degrades to release at least one metabolite. Thus, in various embodiments, the therapeutic agent is a metabolite. In some embodiments, the metabolite is a carbon-center metabolite.

In one embodiment, the nanoparticle further comprises a targeting domain. In one aspect, the nanoparticle further comprises a targeting domain attached to the surface of the nanoparticle. In some embodiments, the targeting domain is bound to an exterior surface of the nanoparticle and recognizes a particular site of interest in a subject. In one embodiment, the targeting domain binds to at least one associated with a disease or a disorder. In various embodiments, the targeting domain is an antibody, an antibody fragment, a peptide sequence, aptamer, folate, a ligand, a gene component, or any combination thereof. Examples of targeting domains include, but are not limited to antibodies, lymphokines, cytokines, receptor proteins such as CD4 and CD8, solubilized receptor proteins such as soluble CD4, hormones, growth factors, peptidomimetics, synthetic ligands, and the like which specifically bind desired target cells, and nucleic acids which bind corresponding nucleic acids through base pair complementarity. Targeting domains of particular interest include peptidomimetics, peptides, antibodies (e.g., monoclonal antibodies, polyclonal antibodies, recombinant antibodies, human antibodies, humanized antibodies, etc.) and antibody fragments (e.g., the Fab′ fragment).

Methods of making and using antibodies are well known in the art. For example, polyclonal antibodies useful in the present invention are generated by immunizing rabbits according to standard immunological techniques well-known in the art. Such techniques include immunizing an animal with a chimeric protein comprising a portion of another protein such as a maltose binding protein or glutathione (GSH) tag polypeptide portion, and/or a moiety such that the antigenic protein of interest is rendered immunogenic (e.g., an antigen of interest conjugated with keyhole limpet hemocyanin, KLH) and a portion comprising the respective antigenic protein amino acid residues.

However, the invention should not be construed as being limited solely to methods and compositions including these antibodies or to these portions of the antigens. Rather, the invention should be construed to include other antibodies, as that term is defined elsewhere herein, to antigens, or portions thereof. Further, the present invention should be construed to encompass antibodies, inter alia, which bind to the specific antigens of interest.

One skilled in the art would appreciate, based upon the disclosure provided herein, that the antibody can specifically bind with any portion of an antigen target, which can be used to generate antibodies specific therefor. However, the present invention is not limited to using the full-length protein as an immunogen. Rather, the present invention includes using an immunogenic portion of the protein to produce an antibody that specifically binds with a specific antigen. That is, the invention includes immunizing an animal using an immunogenic portion, or antigenic determinant, of the antigen.

The antibodies can be produced by immunizing an animal such as, but not limited to, a rabbit, a mouse or a camel, with an antigenic protein of the invention, or a portion thereof, by immunizing an animal using a protein comprising at least a portion of the antigen, or a fusion protein including a tag polypeptide portion comprising, for example, a maltose binding protein tag polypeptide portion, covalently linked with a portion comprising the appropriate amino acid residues. One skilled in the art would appreciate, based upon the disclosure provided herein, that smaller fragments of these proteins can also be used to produce antibodies that specifically bind the antigen of interest.

Once armed with the sequence of a specific antigen of interest and the detailed analysis localizing the various conserved and non-conserved domains of the protein, the skilled artisan would understand, based upon the disclosure provided herein, how to obtain antibodies specific for the various portions of the antigen using methods well-known in the art or to be developed.

Further, the skilled artisan, based upon the disclosure provided herein, would appreciate that using a non-conserved immunogenic portion can produce antibodies specific for the non-conserved region thereby producing antibodies that do not cross-react with other proteins which can share one or more conserved portions. Thus, one skilled in the art would appreciate, based upon the disclosure provided herein, that the non-conserved regions of an antigen of interest can be used to produce antibodies that are specific only for that antigen and do not cross-react non-specifically with other proteins.

The invention encompasses monoclonal, synthetic antibodies, and the like. One skilled in the art would understand, based upon the disclosure provided herein, that the crucial feature of the antibody of the invention is that the antibody bind specifically with an antigen of interest. That is, the antibody of the invention recognizes an antigen of interest or a fragment thereof (e.g., an immunogenic portion or antigenic determinant thereof).

The skilled artisan would appreciate, based upon the disclosure provided herein, that present invention includes use of a single antibody recognizing a single antigenic epitope but that the invention is not limited to use of a single antibody. Instead, the invention encompasses use of at least one antibody where the antibodies can be directed to the same or different antigenic protein epitopes.

The generation of polyclonal antibodies is accomplished by inoculating the desired animal with the antigen and isolating antibodies which specifically bind the antigen therefrom using standard antibody production methods such as those described in, for example, Harlow et al. (1988, In: Antibodies, A Laboratory Manual, Cold Spring Harbor, N.Y.).

Monoclonal antibodies directed against full length or peptide fragments of a protein or peptide may be prepared using any well-known monoclonal antibody preparation procedures, such as those described, for example, in Harlow et al. (1988, In: Antibodies, A Laboratory Manual, Cold Spring Harbor, N.Y.) and in Tuszynski et al. (1988, Blood, 72:109-115). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

Nucleic acid encoding the monoclonal antibody obtained using the procedures described herein may be cloned and sequenced using technology which is available in the art, and is described, for example, in Wright et al. (1992, Critical Rev. Immunol. 12:125-168), and the references cited therein. Further, the antibody of the invention may be “humanized” using the technology described in, for example, Wright et al., and in the references cited therein, and in Gu et al. (1997, Thrombosis and Hematocyst 77:755-759), and other methods of humanizing antibodies well-known in the art or to be developed.

In some embodiments, a non-human antibody is humanized, where specific sequences or regions of the antibody are modified to increase similarity to an antibody naturally produced in a human or fragment thereof. A humanized antibody can be produced using a variety of techniques known in the art, including but not limited to, CDR-grafting (see, e.g., European Patent No. EP 239,400; International Publication No. WO 91/09967; and U.S. Pat. Nos. 5,225,539, 5,530,101, and 5,585,089), veneering or resurfacing (see, e.g., European Patent Nos. EP 592,106 and EP 519,596; Padlan, 1991, Molecular Immunology, 28(4/5):489-498; Studnicka et al., 1994, Protein Engineering, 7(6):805-814; and Roguska et al., 1994, PNAS, 91:969-973), chain shuffling (see, e.g., U.S. Pat. No. 5,565,332), and techniques disclosed in, e.g., U.S. Patent Application Publication No. US2005/0042664, U.S. Patent Application Publication No. US2005/0048617, U.S. Pat. Nos. 6,407,213, 5,766,886, International Publication No. WO 9317105, Tan et al., J. Immunol., 169:1119-25 (2002), Caldas et al., Protein Eng., 13(5):353-60 (2000), Morea et al., Methods, 20(3):267-79 (2000), Baca et al., J. Biol. Chem., 272(16):10678-84 (1997), Roguska et al., Protein Eng., 9(10):895-904 (1996), Couto et al., Cancer Res., 55 (23 Supp):5973s-5977s (1995), Couto et al., Cancer Res., 55(8):1717-22 (1995), Sandhu J S, Gene, 150(2):409-10 (1994), and Pedersen et al., J. Mol. Biol., 235(3):959-73 (1994). Often, framework residues in the framework regions will be substituted with the corresponding residue from the CDR donor antibody to alter, for example improve, antigen binding. These framework substitutions are identified by methods well-known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature, 332:323.)

In one embodiment, the antibody fragment provided herein is a single chain variable fragment (scFv). In various embodiments, the antibodies of the invention may exist in a variety of other forms including, for example, Fv, Fab, and (Fab′) 2, as well as bi-functional (i.e. bi-specific) hybrid antibodies (e.g., Lanzavecchia et al., Eur. J. Immunol. 17, 105 (1987)). In some embodiments, the antibodies and fragments thereof of the invention bind a cell bearing antigen, TCR, and/or BCR with wild-type or enhanced affinity. In some embodiments, the antibodies and fragments thereof of the invention bind a T cell bearing TCR with wild-type or enhanced affinity. In some embodiments, the antibodies and fragments thereof of the invention bind a B cell bearing BCR with wild-type or enhanced affinity. In various embodiments, a human scFv may also be derived from a yeast display library.

ScFvs can be prepared according to method known in the art (see, for example, Bird et al., (1988) Science 242:423-426 and Huston et al., (1988) Proc. Natl. Acad. Sci. USA 85:5879-5883). ScFv molecules can be produced by linking VH and VL regions together using flexible polypeptide linkers. The scFv molecules comprise flexible polypeptide linker (e.g., a Ser-Gly linker) with an optimized length and/or amino acid composition. The flexible polypeptide linker length can greatly affect how the variable regions of an scFv fold and interact. In fact, if a short polypeptide linker is employed (e.g., between 5-10 amino acids, intrachain folding is prevented. Interchain folding is also required to bring the two variable regions together to form a functional epitope binding site. For examples of linker orientation and size see, e.g., Hollinger et al. 1993 Proc Natl Acad. Sci. U.S.A. 90:6444-6448, U.S. Patent Application Publication Nos. 2005/0100543, 2005/0175606, 2007/0014794, and PCT publication Nos. WO2006/020258 and WO2007/024715.

The scFv can comprise a polypeptide linker sequence of at least 1, 2, 3, 4, 5, 6, 7, 8, 9, 10, 11, 12, 13, 14, 15, 16, 17, 18, 19, 20, 25, 30, 35, 40, 45, 50, or more amino acid residues between its VL and VH regions. The flexible polypeptide linker sequence may comprise any naturally occurring amino acid. In some embodiments, the flexible polypeptide linker sequence comprises amino acids glycine and serine. In another embodiment, the flexible polypeptide linker sequence comprises sets of glycine and serine repeats such as (Gly4Ser)n, where n is a positive integer equal to or greater than 1. In one embodiment, the flexible polypeptide linkers include, but are not limited to, (Gly4Ser)4 or (Gly4Ser)3. Variation in the flexible polypeptide linker length may retain or enhance activity, giving rise to superior efficacy in activity studies.

In one embodiment, the targeting domain is bound directly to the nanoparticle. In one embodiment, the targeting domain is bound directly to the surface of the nanoparticle. In one embodiment, the targeting domain is bound to the nanoparticle using a linking molecule. In one embodiment, the targeting domain is bound to the surface of the nanoparticle using a linking molecule. The linking molecules useful in the compositions and methods of the present disclosure may be any molecule capable of binding to both the nanoparticle and the targeting domains used in the compositions and methods of the present disclosure. In certain embodiments, the linking molecule may be a hydrophilic polymer. Examples of linking molecules include, but are not limited to, poly(ethylene glycol) and its derivatives, dithiol compounds, dithiol compounds with hydrazide and/or carboxylic functionality, or single thiols and/or amines or their derivatives.

In certain embodiments, the linking molecule and the targeting domain may be bound by one or more covalent bonds. In certain embodiments, the linking molecule, in addition to linking the targeting domain and the nanoparticle, may impart certain benefits upon the compositions of the present disclosure, including, but not limited to, improved hydrophilicity and stability in solution, reduced immunogenic responses upon introduction of the compositions of the present disclosure into a subject, increased circulation time of the compositions of the present disclosure when introduced into the bloodstream of a subject. The choice of a linking molecule may depend upon, among other things, the targeting domain chosen and the subject into which the compositions of the present invention are to be introduced. One of ordinary skill in the art, with the benefit of this disclosure, will recognize additional suitable linking molecules. Such linking molecules are considered to be within the spirit of the present disclosure.

In certain embodiments, the targeting domain may recognize a particular ligand or receptor present in a desired cell and/or tissue type when introduced into a subject. In certain embodiments, the targeting domain may be an antibody that recognizes such a particular ligand or receptor. The use of antibody fragments may also be suitable in the compositions of the present disclosure. The choice of a targeting domain may depend upon, among other things, the cell and/or tissue type into which an at least partial increase in uptake of the compositions of the present disclosure is desired, as well as particular ligand(s) present in such cell and/or tissue types.

In certain embodiments, the targeting domain may be chosen, among other things, to at least partially increase the uptake of the nanoparticle of the present disclosure into a desired cell and/or tissue type when introduced into a subject.

In some embodiments, the suitable targeting domain may be a peptide sequence, DNA fragment, aptamer, RNA, folate, polymer, etc. One of ordinary skill in the art, with the benefit of this disclosure, will recognize other targeting domains that may be useful in the compositions of the present disclosure. Such targeting domains are considered to be within the spirit of the present disclosure.

To obtain additional selectivity, the nanoparticle may be passively or actively targeted to regions of interest, such as organs, vessels, sites of disease, wounds, or a specific organism in a subject. In active targeting, the nanoparticle may be attached to biological recognition agents to allow them to accumulate in or to be selectively retained by or to be slowly eliminated from certain parts of the body, such as specific organs, parts of organs, bodily structures and disease structures and lesions. Active targeting is defined as a modification of biodistribution using chemical groups that will associate with species present in the desired tissue or organism to effectively decrease the rate of loss of nanoparticle from the specific tissue or organism.

Active targeting of the nanoparticle can be considered as localization through modification of biodistribution of the nanoparticle by means of a targeting domain that is attached to or incorporated into the nanoparticle. The targeting domain can associate or bind with one or more receptor species present in the tissue or organism of interest. This binding will effectively decrease the rate of loss of nanoparticle from the specific tissue or organism of interest. In such cases, the nanoparticle can be modified synthetically to incorporate the targeting domain. Targeted nanoparticle can localize because of binding between the ligand and the targeted receptor. Alternatively, the nanoparticle can distribute by passive biodistribution, i.e., by passive targeting, into diseased tissues of interest such as wounds. Thus, even without synthetic manipulation to incorporate a targeting domain that can bind to a receptor site, passively targeted contrast agents can accumulate in a diseased tissue or in specific locations in the subject, such as the skin. The present invention comprises use of a nanoparticle that is linked to a targeting domain that has an affinity for binding to a receptor. For example, the receptor is located on the surface of a diseased cell or wounded tissue in a human or animal subject.

In some embodiments, the nanoparticle further comprises a biocompatible metal. Examples of biocompatible metals include, but are not limited to, copper, iron oxide, cobalt and noble metals, such as gold and/or silver. One of ordinary skill in the art will be able to select a suitable type of nanoparticle taking into consideration at least the type of imaging and/or therapy to be performed.

In various embodiments, the particle further comprises at least one calcium. For example, in some embodiments, the particle comprises calcium and phosphorous in a ratio of between about 2:about 1 to about 3:about 1.

### Compositions

The present invention also provides various compositions comprising the polymers and/or particles of the present invention. In one embodiment, the composition is a biodegradable composition. In one embodiment, the composition is a medical biodegradable composition.

In some embodiments, the composition comprises: one or more polymers of the present invention and at least one glycolytic inhibitor. In some embodiments, the at least one glycolytic inhibitor is encapsulated in the particle of the present invention. In some embodiments, the at least one glycolytic inhibitor is any glycolytic inhibitor known in the art. For example, in one embodiment, the at least one glycolytic inhibitor is PFK15.

In one embodiment, the present invention provides an immunogenic composition for inducing an immune response against a cancer in a subject. For example, in one embodiment, the immunogenic composition is a vaccine. In some embodiments, the composition induces an immune response against a cancer antigen in a cell, tissue or subject. In some instances, the vaccine induces a protective immune response in the subject.

A vaccine of the present invention may vary in its composition of nucleic acid and/or amino acid. In one embodiment, the vaccine comprises a nucleic acid. In one embodiment, the vaccine comprises a nucleic acid encoding an antigen or a fragment or variant thereof. In one embodiment, the vaccine comprises an amino acid. In a non-limiting example, a vaccine might also be formulated with an adjuvant. Of course, it will be understood that various compositions described herein may further comprise additional components. For example, one or more vaccine components may be comprised in a lipid, liposome, or lipid particle. In another non-limiting example, a vaccine may comprise one or more adjuvants. A vaccine of the present invention, and its various components, may be prepared and/or administered by any method disclosed herein or as would be known to one of ordinary skill in the art, in light of the present disclosure.

In various aspects, the composition comprises: one or more polymers of the present invention and one or more stabilizers. In other aspects, the composition comprises: one or more particles of the present invention and one or more stabilizers. In various embodiments, the stabilizer to particle weight ratio is less than 50%. In one embodiment, the stabilizer comprises a biocompatible polymer. Examples of stabilizers include, but are not limited to, biocompatible polymer, a biodegradable polymer, a multifunctional linker, starch, modified starch, and starch derivatives, gums, including but not limited to polymers, polypeptides, albumin, amino acids, thiols, amines, carboxylic acid and combinations or derivatives thereof, citric acid, xanthan gum, alginic acid, other alginates, benitoniite, veegum, agar, guar, locust bean gum, gum arabic, quince psyllium, flax seed, okra gum, arabinoglactin, pectin, tragacanth, scleroglucan, dextran, amylose, amylopectin, dextrin, etc., cross-linked polyvinylpyrrolidone, ion-exchange resins, potassium polymethacrylate, carrageenan (and derivatives), gum karaya and biosynthetic gum, polycarbonates (linear polyesters of carbonic acid); microporous materials (bisphenol, a microporous poly(vinylchloride), micro-porous polyamides, microporous modacrylic copolymers, microporous styrene-acrylic and its copolymers); porous polysulfones, halogenated poly(vinylidene), polychloroethers, acetal polymers, polyesters prepared by esterification of a dicarboxylic acid or anhydride with an alkylene polyol, poly(alkylenesulfides), phenolics, polyesters, asymmetric porous polymers, cross-linked olefin polymers, hydrophilic microporous homopolymers, copolymers or interpolymers having a reduced bulk density, and other similar materials, poly(urethane), cross-linked chain-extended poly(urethane), poly(imides), poly(benzimidazoles), collodion, regenerated proteins, semi-solid cross-linked poly(vinylpyrrolidone), monomeric, dimeric, oligomeric or long-chain, copolymers, block polymers, block co-polymers, polymers, PEG, dextran, modified dextran, polyvinylalcohol, polyvinylpyrollidone, polyacrylates, polymethacrylates, polyanhydrides, polypeptides, albumin, alginates, amino acids, thiols, amines, carboxylic acids, or combinations thereof.

In various embodiments, the composition further comprises particles dispersed in the organic liquid. In some embodiments, the composition comprises an organic liquid comprising a plurality of particles of the present invention dispersed therein, and a coating material disposed around the exterior surface of the organic liquid. In one embodiment, the composition comprises an organic liquid and particles dispersed in organic liquid. In some embodiments, the composition further comprises a coating, which surrounds the exterior surface of organic liquid. Examples of suitable coating materials may include, but are not limited to bovine serum albumin (BSA), lipids, polymers, and combinations thereof. Examples of organic liquids suitable for use in the particle cluster composition of the present disclosure may include, but are not limited to, perfluorocarbons, such as perfluorocarbons comprising about 5 to about 12 carbons, dodecafluoropentane (DDFP), commercially available from FluoroMed, L. P., Round Rock, Tex., and perfluororpentane.

The compositions are formulated in a pharmaceutically acceptable excipient, such as wetting agents, buffers, disintegrants, binders, fillers, flavoring agents and liquid carrier media such as sterile water, water/ethanol etc. The compositions should be suitable for administration either by topical administration or injection or inhalation or catheterization or instillation or transdermal introduction into any of the various body cavities including the alimentary canal, the vagina, the rectum, the bladder, the ureter, the urethra, the mouth, etc. For oral administration, the pH of the composition is, for example, in the acid range (e.g., 2 to 7) and buffers or pH adjusting agents may be used. The contrast media may be formulated in conventional pharmaceutical administration forms, such as tablets, capsules, powders, solutions, dispersion, syrups, suppositories etc.

The compounds, particles, or compositions of the invention can be formulated and administered to a subject, as now described. The invention encompasses the preparation and use of pharmaceutical compositions comprising the compound, particle, and/or compositions of the invention useful for the delivery of a therapeutic agent, such as metabolite, to a cell (e.g., delivery of succinate to a dendritic cell). The invention also encompasses the preparation and use of pharmaceutical compositions comprising the compound, particle, and/or compositions of the invention useful for the treatment of a disease or disorder (e.g., any disease or disorder associated with increased level of a pro-inflammatory cytokine; decreased level of an anti-inflammatory cytokine; decreased level of a T regulatory cell; or any combination thereof). The invention also encompasses the preparation and use of pharmaceutical compositions comprising the compound, particle, and/or compositions of the invention useful for the growth or regeneration of biological tissue (e.g., wound healing).

Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

The pharmaceutical compositions useful for practicing the invention may be administered to deliver a dose of between about 0.01 ng/kg/day and 500 mg/kg/day.

In various embodiments, the pharmaceutical compositions useful in the methods of the invention may be administered, by way of example, systemically, parenterally, or topically, such as, in oral formulations, inhaled formulations, including solid or aerosol, and by topical or other similar formulations. In addition to the appropriate therapeutic composition, such pharmaceutical compositions may contain pharmaceutically acceptable carriers and other ingredients known to enhance and facilitate drug administration. Other possible formulations, such as particles, liposomes, resealed erythrocytes, and immunologically based systems may also be used to administer an appropriate modulator thereof, according to the methods of the invention.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals, patients, and subjects of all sorts. Modification of pharmaceutical compositions suitable for administration to humans in order to render the compositions suitable for administration to various animals and patients is well understood, and the ordinarily skilled veterinary pharmacologist can design and perform such modification with merely ordinary, if any, experimentation.

Pharmaceutical compositions that are useful in the methods of the invention may be prepared, packaged, or sold in formulations suitable for oral, rectal, vaginal, parenteral, topical, pulmonary, intranasal, buccal, intravenous, ophthalmic, intrathecal and other known routes of administration. Other contemplated formulations include projected particles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents.

Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycolate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and U.S. Pat. No. 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent.

Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, and hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, cutaneous, subcutaneous, intraperitoneal, intravenous, intramuscular, intracisternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and, for example, from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. For example, such powders comprise particles wherein at least 98% of the particles by weight have a diameter greater than 0.5 nanometers and at least 95% of the particles by number have a diameter less than 7 nanometers. For example, at least 95% of the particles by weight have a diameter greater than 1 nanometer and at least 90% of the particles by number have a diameter less than 6 nanometers. Dry powder compositions, for example, include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (for example, having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration, for example, have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers.

Such a formulation is administered in the manner in which snuff is taken i.e. by rapid inhalation through the nasal passage from a container of the powder held close to the nares. Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, contain 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, for example, have an average particle or droplet size in the range from about 0.1 nanomaters to about 2000 micrometers, and may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1-1.0% (w/w) solution or suspension of the active ingredient in an aqueous or oily liquid carrier. Such drops may further comprise buffering agents, salts, or one or more other of the additional ingredients described herein. Other opthalmically-administrable formulations which are useful include those which comprise the active ingredient in microcrystalline form or in a liposomal preparation.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa.

Typically dosages of the compound of the invention which may be administered to an animal or patient, such as a human, range in amount from about 0.01 mg to about 100 g per kilogram of body weight of the animal or patient. While the precise dosage administered will vary depending upon any number of factors, including, but not limited to, the type of animal and type of disease state being treated, the age of the animal or patient and the route of administration. For example, the dosage of the compound will vary from about 0.01 mg to about 500 mg per kilogram of body weight of the animal or patient. The compound can be administered to an animal or patient as frequently as several times daily, or it can be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, patient, etc.

Administration of the compounds of the present invention or the compositions thereof may be continuous or intermittent, depending, for example, upon the recipient's physiological condition, whether the purpose of the administration is therapeutic or prophylactic, and other factors known to skilled practitioners. The administration of the agents of the invention may be essentially continuous over a preselected period of time or may be in a series of spaced doses. Both local and systemic administration is contemplated. The amount administered will vary depending on various factors including, but not limited to, the composition chosen, the particular disease, the weight, the physical condition, and the age of the mammal, and whether prevention or treatment is to be achieved. Such factors can be readily determined by the clinician employing animal models or other test systems which are well known to the art.

One or more suitable unit dosage forms having the therapeutic agent(s) of the invention, which, as discussed below, may optionally be formulated for sustained release, can be administered by a variety of routes including parenteral, including by intravenous and intramuscular routes, as well as by direct injection into the diseased tissue. For example, the therapeutic agent may be directly injected into the muscle. The formulations may, where appropriate, be conveniently presented in discrete unit dosage forms and may be prepared by any of the methods well known to pharmacy. Such methods may include the step of bringing into association the therapeutic agent with liquid carriers, solid matrices, semi-solid carriers, finely divided solid carriers or combinations thereof, and then, if necessary, introducing or shaping the product into the desired delivery system.

When the therapeutic agents of the invention are prepared for administration, they are, for example, combined with a pharmaceutically acceptable carrier, diluent or excipient to form a pharmaceutical formulation, or unit dosage form. The total active ingredients in such formulations include from 0.1 to 99.9% by weight of the formulation. A “pharmaceutically acceptable” is a carrier, diluent, excipient, and/or salt that is compatible with the other ingredients of the formulation, and not deleterious to the recipient thereof. The active ingredient for administration may be present as a powder or as granules; as a solution, a suspension or an emulsion.

Pharmaceutical formulations containing the therapeutic agents of the invention can be prepared by procedures known in the art using well known and readily available ingredients. The therapeutic agents of the invention can also be formulated as solutions appropriate for parenteral administration, for instance by intramuscular, subcutaneous or intravenous routes.

The pharmaceutical formulations of the therapeutic agents of the invention can also take the form of an aqueous or anhydrous solution or dispersion, or alternatively the form of an emulsion or suspension.

Thus, the therapeutic agent may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

It will be appreciated that the unit content of active ingredient or ingredients contained in an individual aerosol dose of each dosage form need not in itself constitute an effective amount for treating the particular indication or disease since the necessary effective amount can be reached by administration of a plurality of dosage units. Moreover, the effective amount may be achieved using less than the dose in the dosage form, either individually, or in a series of administrations.

The pharmaceutical formulations of the present invention may include, as optional ingredients, pharmaceutically acceptable carriers, diluents, solubilizing or emulsifying agents, and salts of the type that are well-known in the art. Specific non-limiting examples of the carriers and/or diluents that are useful in the pharmaceutical formulations of the present invention include water and physiologically acceptable buffered saline solutions, such as phosphate buffered saline solutions pH 7.0-8.0.

In general, water, suitable oil, saline, aqueous dextrose (glucose), and related sugar solutions and glycols such as propylene glycol or polyethylene glycols are suitable carriers for parenteral solutions. Solutions for parenteral administration contain the active ingredient, suitable stabilizing agents and, if necessary, buffer substances. Antioxidizing agents such as sodium bisulfate, sodium sulfite or ascorbic acid, either alone or combined, are suitable stabilizing agents. Also used are citric acid and its salts and sodium Ethylenediaminetetraacetic acid (EDTA). In addition, parenteral solutions can contain preservatives such as benzalkonium chloride, methyl- or propyl-paraben and chlorobutanol. Suitable pharmaceutical carriers are described in Remington's Pharmaceutical Sciences, a standard reference text in this field.

The active ingredients of the invention may be formulated to be suspended in a pharmaceutically acceptable composition suitable for use in mammals and in particular, in humans. Such formulations include the use of adjuvants such as muramyl dipeptide derivatives (MDP) or analogs that are described in U.S. Pat. Nos. 4,082,735; 4,082,736; 4,101,536; 4,185,089; 4,235,771; and 4,406,890. Other adjuvants, which are useful, include alum (Pierce Chemical Co.), lipid A, trehalose dimycolate and dimethyldioctadecylammonium bromide (DDA), Freund's adjuvant, and IL-12. Other components may include a polyoxypropylene-polyoxyethylene block polymer (Pluronic®), a non-ionic surfactant, and a metabolizable oil such as squalene (U.S. Pat. No. 4,606,918).

Additionally, standard pharmaceutical methods can be employed to control the duration of action. These are well known in the art and include control release preparations and can include appropriate macromolecules, for example polymers, polyesters, polyamino acids, polyvinyl, pyrolidone, ethylenevinylacetate, methyl cellulose, carboxymethyl cellulose or protamine sulfate. The concentration of macromolecules as well as the methods of incorporation can be adjusted in order to control release. Additionally, the agent can be incorporated into particles of polymeric materials such as polyesters, polyamino acids, hydrogels, poly(lactic acid) or ethylenevinylacetate copolymers. In addition to being incorporated, these agents can also be used to trap the compound in microcapsules.

Accordingly, the composition of the present invention may be delivered via various routes and to various sites in a mammal body to achieve a particular effect (see, e.g., Rosenfeld et al., 1991; Rosenfeld et al., 1991a; Jaffe et al., supra; Berkner, supra). One skilled in the art will recognize that although more than one route can be used for administration, a particular route can provide a more immediate and more effective reaction than another route. In one embodiment, the composition described above is administered to the subject by subretinal injection. In other embodiments, the composition is administered by intravitreal injection. Other forms of administration that may be useful in the methods described herein include, but are not limited to, direct delivery to a desired organ (e.g., the eye), oral, inhalation, intranasal, intratracheal, intravenous, intramuscular, subcutaneous, intradermal, and other parental routes of administration. Additionally, routes of administration may be combined, if desired. In another embodiments, route of administration is subretinal injection or intravitreal injection.

The active ingredients of the present invention can be provided in unit dosage form wherein each dosage unit, e.g., a teaspoonful, tablet, solution, or suppository, contains a predetermined amount of the composition, alone or in appropriate combination with other active agents. The term “unit dosage form” as used herein refers to physically discrete units suitable as unitary dosages for human and mammal subjects, each unit containing a predetermined quantity of the compositions of the present invention, alone or in combination with other active agents, calculated in an amount sufficient to produce the desired effect, in association with a pharmaceutically acceptable diluent, carrier, or vehicle, where appropriate. The specifications for the unit dosage forms of the present invention depend on the particular effect to be achieved and the particular pharmacodynamics associated with the composition in the particular host.

These methods described herein are by no means all-inclusive, and further methods to suit the specific application will be apparent to the ordinary skilled artisan. Moreover, the effective amount of the compositions can be further approximated through analogy to compounds known to exert the desired effect.

It will be understood by those of skill in the art that numerous and various modifications can be made without departing from the spirit of the present disclosure. Therefore, it should be clearly understood that the forms disclosed herein are illustrative only and are not intended to limit the scope of the present disclosure.

### Methods of Prevention, Treatment, and Immunotherapy

The polymers, particles, and/or compositions thereof of the present invention can be used to modulate the function of a cell (e.g., immune cell). Thus, in one embodiment, the present invention provides a method to induce an immune response. In one embodiment, the present invention provides a method to induce a metabolism of a cell (e.g., immune cell). In one embodiment, the present invention provides a method to induce an activation of a cell (e.g., immune cell). In one embodiment, the present invention provides a method to induce glycolysis. In one embodiment, the present invention provides a method to induce a TCA cycle. In one embodiment, the present invention provides a method to induce a polyamine pathway. In one embodiment, the present invention provides a method to induce a PPP. In one embodiment, the present invention provides a method to induce an ECAR. In one embodiment, the present invention provides a method to induce an OCR. In one embodiment, the present invention provides a method to induce a mitochondrial respiration. In one embodiment, the present invention provides a method to induce a release of a metabolite in a cell (e.g., immune cell). In one embodiment, the present invention provides a method to induce a pro-inflammatory response. In one embodiment, the present invention provides a method to induce one or more BRAF inhibitors. In one embodiment, the present invention provides a method to induce one or more tyrosine kinase inhibitors. In one embodiment, the present invention provides a method to induce one or more ERK inhibitors. In one embodiment, the present invention provides a method to induce one or more MEK inhibitors. In one embodiment, the present invention provides a method to induce a cancer cell suppression. In one embodiment, the present invention provides a method to reduce a cancer cell proliferation. In one embodiment, the present invention provides a T cell therapy. In one embodiment, the present invention provides an adoptive T cell therapy.

Thus, in various embodiments, the present invention provides a method of treating or preventing a disease or disorder associated with cell function, immune cell function, metabolic inhibition, immune response, activation of a cell, activation of an immune cell, glycolysis, TCA cycle, polyamine pathway, PPP, ECAR, OCR, mitochondrial respiration, level of a metabolite in a cell, pro-inflammatory response, a BRAF inhibitor, tyrosine kinase inhibitor, ERK inhibitor, MEK inhibitor, or any combination thereof in a subject in need thereof. In one embodiment, the present invention provides a method of preventing or treating a metabolic inhibition of a cell (e.g., immune cell) in a subject in need thereof. In one embodiment, the present invention provides a method of treating or preventing a disease or disorder in a subject in need thereof. In one embodiment, the disease or disorder is a disease or disorder associated with abnormal immune cell function in a subject in need thereof. In one embodiment, the present invention provides a method of treating or preventing a cancer in a subject in need thereof.

Exemplary tumors and types of cancer treatable by way of the present invention includes, but is not limited to, bone cancer, such as Ewing Sarcoma, pancreatic cancer, pancreatic ductal adenocarcinoma (PDA), colon cancer, colorectal adenocarcinoma, gastric adenocarcinoma and cervical adenocarcinoma, renal papillary cancer, prostate cancer, and small intestine neuroendocrine tumor (NET). The following are non-limiting examples of cancers that can be treated by the disclosed methods and compositions: acute lymphoblastic; acute myeloid leukemia; adrenocortical carcinoma; adrenocortical carcinoma, childhood; appendix cancer; basal cell carcinoma; bile duct cancer, extrahepatic; bladder cancer; bone cancer; osteosarcoma and malignant fibrous histiocytoma; brain stem glioma, childhood; brain tumor, adult; brain tumor, brain stem glioma, childhood; brain tumor, central nervous system atypical teratoid/rhabdoid tumor, childhood; central nervous system embryonal tumors; cerebellar astrocytoma; cerebral astrocytotna/malignant glioma; craniopharyngioma; ependymoblastoma; ependymoma; medulloblastoma; medulloepithelioma; pineal parenchymal tumors of intermediate differentiation; supratentorial primitive neuroectodermal tumors and pineoblastoma; visual pathway and hypothalamic glioma; brain and spinal cord tumors; breast cancer; bronchial tumors; Burkitt lymphoma; carcinoid tumor; carcinoid tumor, gastrointestinal; central nervous system atypical teratoid/rhabdoid tumor; central nervous system embryonal tumors; central nervous system lymphoma; cerebellar astrocytoma cerebral astrocytoma/malignant glioma, childhood; cervical cancer; chordoma, childhood; chronic lymphocytic leukemia; chronic myelogenous leukemia; chronic myeloproliferative disorders; colon cancer; colorectal cancer; craniopharyngioma; cutaneous T-cell lymphoma; esophageal cancer; Ewing family of tumors; extragonadal germ cell tumor; extrahepatic bile duct cancer; eye cancer, intraocular melanoma; eye cancer, retinoblastoma; gallbladder cancer; gastric (stomach) cancer; gastrointestinal carcinoid tumor; gastrointestinal stromal tumor (gist); germ cell tumor, extracranial; germ cell tumor, extragonadal; germ cell tumor, ovarian; gestational trophoblastic tumor; glioma; glioma, childhood brain stem; glioma, childhood cerebral astrocytoma; glioma, childhood visual pathway and hypothalamic; hairy cell leukemia; head and neck cancer; hepatocellular (liver) cancer; histiocytosis, Langerhans cell; Hodgkin lymphoma; hypopharyngeal cancer; hypothalamic and visual pathway glioma; intraocular melanoma; islet cell tumors; kidney (renal cell) cancer; Langerhans cell histiocytosis; laryngeal cancer; leukemia, acute lymphoblastic; leukemia, acute myeloid; leukemia, chronic lymphocytic; leukemia, chronic myelogenous; leukemia, hairy cell; lip and oral cavity cancer; liver cancer; lung cancer, non-small cell; lung cancer, small cell; lymphoma, aids-related; lymphoma, Burkitt; lymphoma, cutaneous T-cell; lymphoma, non-Hodgkin lymphoma; lymphoma, primary central nervous system; macroglobulinemia, Waldenstrom; malignant fibrous histiocvtoma of bone and osteosarcoma; medulloblastoma; melanoma; melanoma, intraocular (eye); Merkel cell carcinoma; mesothelioma; metastatic squamous neck cancer with occult primary; mouth cancer; multiple endocrine neoplasia syndrome, (childhood); multiple myeloma/plasma cell neoplasm; mycosis; fungoides; myelodysplastic syndromes; myelodysplastic/myeloproliferative diseases; myelogenous leukemia, chronic; myeloid leukemia, adult acute; myeloid leukemia, childhood acute; myeloma, multiple; myeloproliferative disorders, chronic; nasal cavity and paranasal sinus cancer; nasopharyngeal cancer; neuroblastoma; non-small cell lung cancer; oral cancer; oral cavity cancer; oropharyngeal cancer; osteosarcoma and malignant fibrous histiocytoma of bone; ovarian cancer; ovarian epithelial cancer; ovarian germ cell tumor; ovarian low malignant potential tumor; pancreatic cancer; pancreatic cancer, islet cell tumors; papillomatosis; parathyroid cancer; penile cancer; pharyngeal cancer; pheochromocytoma; pineal parenchymal tumors of intermediate differentiation; pineoblastoma and supratentorial primitive neuroectodermal tumors; pituitary tumor; plasma celt neoplasm/multiple myeloma; pleuropulmonary blastoma; primary central nervous system lymphoma; prostate cancer; rectal cancer; renal cell (kidney) cancer; renal pelvis and ureter, transitional cell cancer; respiratory tract carcinoma involving the nut gene on chromosome 15; retinoblastoma; rhabdomyosarcoma; salivary gland cancer; sarcoma, Ewing family of tumors; sarcoma, Kaposi; sarcoma, soft tissue; sarcoma, uterine; Sezary syndrome; skin cancer (nonmelanoma); skin cancer (melanoma); skin carcinoma, Merkel cell; small cell lung cancer; small intestine cancer; soft tissue sarcoma; squamous cell carcinoma, squamous neck cancer with occult primary, metastatic; stomach (gastric) cancer; supratentorial primitive neuroectodermal tumors; T-cell lymphoma, cutaneous; testicular cancer; throat cancer; thymoma and thymic carcinoma; thyroid cancer; transitional cell cancer of the renal pelvis and ureter; trophoblastic tumor, gestational; urethral cancer; uterine cancer, endometrial; uterine sarcoma; vaginal cancer; vulvar cancer; Waldenstrom macroglobulinemia; and Wilms tumor.

In some embodiments, the method comprises increasing the level of at least one T cell, Tc1, Tc2, Tc17, Th, Th1, Th17, or Teff. In some embodiments, the method comprises decreasing the level of at least one immune suppressive cell, Th2, Treg, Foxp3, Foxp3+ cell, GATA3, or GATA3+ cell. Thus, in one embodiment, the present invention provides a method of treating or preventing a disease or disorder associated with T cells, Tc1, Tc2, Tc17, Th, Th1, Th17, Teff, immune suppressive cell, Th2, Treg, Foxp3, Foxp3+ cell, GATA3, GATA3+ cell, or any combination thereof in a subject in need thereof.

In various aspects, the polymers, particles, and/or compositions thereof of the present invention can be used to modulate the function of a cell (e.g., immune cell) in the presence of one or more metabolic inhibitors, metabolic pathway accelerators, peptide therapeutics, protein therapeutics, and/or enzymes to accelerate metabolism. Thus, in one embodiment, the present invention provides a method to induce an immune response in the presence of one or more metabolic inhibitors. In one embodiment, the present invention provides a method to induce a metabolism of a cell (e.g., immune cell) in the presence of one or more metabolic inhibitors. In one embodiment, the present invention provides a method to induce an activation of a cell (e.g., immune cell) in the presence of one or more metabolic inhibitors. In one embodiment, the present invention provides a method to induce glycolysis in the presence of one or more metabolic inhibitors. In one embodiment, the present invention provides a method to induce a TCA cycle in the presence of one or more metabolic inhibitors. In one embodiment, the present invention provides a method to induce a polyamine pathway in the presence of one or more metabolic inhibitors. In one embodiment, the present invention provides a method to induce a PPP in the presence of one or more metabolic inhibitors. In one embodiment, the present invention provides a method to induce an ECAR in the presence of one or more metabolic inhibitors. In one embodiment, the present invention provides a method to induce an OCR in the presence of one or more metabolic inhibitors. In one embodiment, the present invention provides a method to induce a mitochondrial respiration in the presence of one or more metabolic inhibitors. In one embodiment, the present invention provides a method to induce a release of a metabolite in a cell (e.g., immune cell) in the presence of one or more metabolic inhibitors. In one embodiment, the present invention provides a method to induce a pro-inflammatory response in the presence of one or more metabolic inhibitors. In one embodiment, the present invention provides a method to induce one or more BRAF inhibitors in the presence of one or more metabolic inhibitors. In one embodiment, the present invention provides a method to induce one or more tyrosine kinase inhibitors in the presence of one or more metabolic inhibitors. In one embodiment, the present invention provides a method to induce one or more ERK inhibitors in the presence of one or more metabolic inhibitors. In one embodiment, the present invention provides a method to induce one or more MEK inhibitors in the presence of one or more metabolic inhibitors. In one embodiment, the present invention provides a method to induce a cancer cell suppression in the presence of one or more metabolic inhibitors. In one embodiment, the present invention provides a method to reduce a cancer cell proliferation in the presence of one or more metabolic inhibitors.

Thus, in various embodiments, the present invention provides a method of treating or preventing a disease or disorder associated with immune cell function, metabolic inhibition, immune response, activation of a cell, activation of an immune cell, glycolysis, TCA cycle, polyamine pathway, ECAR, OCR, PPP, ECAR, OCR, mitochondrial respiration, level of a metabolite in a cell (e.g., immune cell), pro-inflammatory response, a BRAF inhibitor, tyrosine kinase inhibitor, ERK inhibitor, MEK inhibitor, or any combination thereof in a subject in need thereof in the presence of one or more metabolic inhibitors, metabolic pathway accelerators, peptide therapeutics, protein therapeutics, and/or enzymes to accelerate metabolism. For example, in one embodiment, the present invention provides a method of preventing or treating a metabolic inhibition of a cell (e.g., immune cell) in a subject in need thereof in the presence of one or more metabolic inhibitors.

In one embodiment, the present invention provides a method of treating or preventing a disease or disorder in a subject in need thereof in the presence of one or more metabolic inhibitors, metabolic pathway accelerators, peptide therapeutics, protein therapeutics, and/or enzymes to accelerate metabolism. For example, in one embodiment, the disease or disorder is a disease or disorder associated with abnormal immune cell function.

In another embodiment, the present invention provides a method of treating or preventing a cancer in a subject in need thereof in the presence of one or more metabolic inhibitors, metabolic pathway accelerators, peptide therapeutics, protein therapeutics, and/or enzymes to accelerate metabolism.

In some embodiments, the method comprises increasing the level of at least one T cell, Tc1, Tc2, Tc17, Th, Th1, Th17, or Teff in the presence of one or more metabolic inhibitors, metabolic pathway accelerators, peptide therapeutics, protein therapeutics, and/or enzymes to accelerate metabolism. In some embodiments, the method comprises decreasing the level of at least one immune suppressive cell, Th2, Treg, Foxp3, Foxp3+ cell, GATA3, or GATA3+ cell in the presence of one or more metabolic inhibitors, metabolic pathway accelerators, peptide therapeutics, protein therapeutics, and/or enzymes to accelerate metabolism. Thus, for example, in one embodiment, the present invention provides a method of treating or preventing a disease or disorder associated with T cells, Tc1, Tc2, Tc17, Th, Th1, Th17, Teff, immune suppressive cell, Th2, Treg, Foxp3, Foxp3+ cell, GATA3, or GATA3+ cell, or any combination thereof in a subject in need thereof in the presence of one or more metabolic inhibitors.

In one embodiment, the method comprises administering at least one polymer described herein to the subject. In one embodiment, the method comprises administering at least one particle described herein to the subject. In one embodiment, the method comprises administering at least one composition described herein to the subject. Thus, in some embodiments, the polymer, particle, or composition of the present invention activates at least one cell (e.g., immune cell). In some embodiments, the polymer, particle, or composition induces an activation of DCs, function of DCs, immune response, activation of a cell, glycolysis, TCA cycle, polyamine pathway, ECAR, OCR, PPP, ECAR, OCR, mitochondrial respiration, level of a metabolite in a cell (e.g., immune cell), pro-inflammatory response, a BRAF inhibitor, tyrosine kinase inhibitor, ERK inhibitor, MEK inhibitor, release of a metabolite in DCs, cancer cell suppression, or any combination thereof. In one embodiment, the polymer, particle, or composition reduces a cancer cell proliferation. In some embodiments, the polymer, particle, or composition increases the level of at least one T cell, Tc1, Tc2, Tc17, Th, Th1, Th17, or Teff. In some embodiments, the polymer, particle, or composition decreases the level of at least one immune suppressive cell, Th2, Treg, Foxp3, Foxp3+ cell, GATA3, or GATA3+ cell.

In one embodiment, the method comprises administering at least one composition described herein to cells and administering the cells to a subject via adoptive cell transfer. In one embodiment, the method comprises administering at least one composition described herein to dendritic cells (DCs) and administering the dendritic cells to a subject via adoptive cell transfer.

In one embodiment, the method comprises administering a particle comprising a compound comprising the structure of Formulae (I)—(XIII), or a derivative or a pharmaceutical acceptable salt thereof, to a subject. In one embodiment, the method comprises administering a particle comprising a compound comprising the structure of Formulae (I)—(XIII), or a derivative or a pharmaceutical acceptable salt thereof, to cells and administering the cells to a subject via adoptive cell transfer. In one embodiment, the particle releases the metabolite from the polymer.

In one embodiment, the metabolite modulates the function of one or more cells. For example, in one embodiment, the metabolite activates immune cells. In one embodiment, the metabolite activates DCs.

The present invention also provides methods of treating a disease or disorder by combining immunotherapy with metabolic inhibition. Thus, in various aspects of the invention, the method further comprises administering a metabolic inhibitor to the subject prior to, simultaneously, or after administering the therapeutically effective amount of the composition to the subject.

Thus, in various aspects of the invention, the method comprises administering at least one polymer described herein to the subject in the presence of one or more metabolic inhibitors, metabolic pathway accelerators, peptide therapeutics, protein therapeutics, and/or enzymes to accelerate metabolism. In one embodiment, the method comprises administering at least one particle described herein to the subject in the presence of one or more metabolic inhibitors, metabolic pathway accelerators, peptide therapeutics, protein therapeutics, and/or enzymes to accelerate metabolism. In one embodiment, the method comprises administering at least one composition described herein to the subject in the presence of one or more metabolic inhibitors, metabolic pathway accelerators, peptide therapeutics, protein therapeutics, and/or enzymes to accelerate metabolism. In one embodiment, the method comprises administering at least one composition described herein to cells and administering the cells to the subject via adoptive cell transfer in the presence of one or more metabolic inhibitors, metabolic pathway accelerators, peptide therapeutics, protein therapeutics, and/or enzymes to accelerate metabolism.

Thus, in some embodiments, the polymer, particle, or composition of the present invention activates at least one cell (e.g., immune cell) in the presence of one or more metabolic inhibitors, metabolic pathway accelerators, peptide therapeutics, protein therapeutics, and/or enzymes to accelerate metabolism. For example, in some embodiments, the polymer, particle, or composition of the present invention activates at least one cell (e.g., immune cell) in the presence of one or more metabolic inhibitors. In some embodiments, the polymer, particle, or composition induces an activation of DCs, function of DCs, immune response, activation of a cell (e.g., immune cell), glycolysis, TCA cycle, polyamine pathway, ECAR, OCR, PPP, ECAR, OCR, mitochondrial respiration, level of a metabolite in a cell (e.g., immune cell), pro-inflammatory response, a BRAF inhibitor, tyrosine kinase inhibitor, ERK inhibitor, MEK inhibitor, release of a metabolite in DCs, cancer cell suppression, or any combination thereof in the presence of one or more metabolic inhibitors.

In one embodiment, the polymer, particle, or composition reduces a cancer cell proliferation in the presence of one or more metabolic inhibitors, metabolic pathway accelerators, peptide therapeutics, protein therapeutics, and/or enzymes to accelerate metabolism.

In some embodiments, the polymer, particle, or composition increases the level of at least one T cell, Tc1, Tc2, Tc17, Th, Th1, Th17, or Teff in the presence of one or more metabolic inhibitors, metabolic pathway accelerators, peptide therapeutics, protein therapeutics, and/or enzymes to accelerate metabolism.

In some embodiments, the polymer, particle, or composition decreases the level of at least one immune suppressive cell, Th2, Treg, Foxp3, Foxp3+ cell, GATA3, or GATA3+ cell in the presence of one or more metabolic inhibitors, metabolic pathway accelerators, peptide therapeutics, protein therapeutics, and/or enzymes to accelerate metabolism.

In some embodiments, the particle delivers an additional therapeutic agent to the subject and/or cells. For example, in one embodiment, the particle encapsulates an additional therapeutic agent and delivers the therapeutic agent to the subject and/or cells.

Thus, in one embodiment, the method of the invention delivers metabolite and an additional therapeutic agent to a subject in need thereof. In one embodiment, the therapeutic agent is any therapeutic agent described herein.

Any therapeutic agent or any combination of therapeutic agents disclosed herein may be administered to a subject to treat a disease or disorder. The therapeutic agents herein can be formulated in any number of ways, often according to various known formulations in the art or as disclosed or referenced herein.

In one embodiment, the invention provides a method for increasing the level of a metabolite in the subject. In one embodiment, the method comprises administering to the subject a particle of the invention. In one embodiment, the method comprises administering to cells a particle of the invention and delivering the cells to a subject via adoptive cell transfer. For example, in one embodiment, the method comprises administering a particle comprising a compound comprising the structure of Formulae (I)—(XIII), or a derivative or a pharmaceutical acceptable salt thereof, wherein the particle releases the metabolite from the polymer. Thus, in one embodiment, the method increases metabolites including, but not limited to, succinic acid, citric acid, isocitric acid, maleic acid, fumaric acid, or any combination thereof.

In some embodiments, the invention provides a method for increasing the level of Th, Th1, Th17, Tc1, Tc17, Teff, Teff to Treg ratio, RORγt, or any combination thereof. In some embodiments, the invention provides a method for decreasing the level of immune suppressive cell, Th2, Treg, Foxp3, Foxp3+ cell, GATA3, GATA3+ cell, or any combination thereof in the subject. Thus, in one embodiment, the present invention provides a method of treating or preventing a disease or disorder associated with decreased level of T cells, Tc1, Tc2, Tc17, Th, Th1, Th17, Teff, or any combination thereof in a subject in need thereof. In one embodiment, the present invention provides a method of treating or preventing a disease or disorder associated with increased level of immune suppressive cell, Th2, Treg, Foxp3, Foxp3+ cell, GATA3, GATA3+ cell, or any combination thereof in a subject in need thereof.

In certain embodiments, the method of treating a disease or disorder comprises a “triggered” functionality. In other words, the system may remain inert in the body until specifically triggered. In some embodiments, the polymer or particle is used advantageously in therapeutic applications such as to first target the polymer or particle to a specified location, and then trigger them into an activated state. Sometimes referred to as a “dual targeted delivery system,” this feature may minimize the side effects of systemic therapeutic agents. For example, in some embodiments, upon delivering the polymer or particle to a specific cell, a reagent, such as water, proton, acid, or protonated water, may be applied to the cell thereby causing the release of a therapeutic agent from the polymer or particle. In some embodiments, this may provide a clinician the ability to control and visualize drug therapy noninvasively.

In some embodiments, the size (e.g., average diameter of the particle) of the compound, particle, or composition of the present invention allows for passive diffusion into cells. In some embodiments, where the compound, particle, or composition is on a smaller scale, the small size (e.g., average diameter of the particle) allows the compound, particle, or compositions to travel almost anywhere in the body where therapy may need to be performed. For example, in some embodiments, the method comprises compounds or particles that act as a hydrolysis triggered therapeutic agent delivery and therapeutic agent release systems.

In some embodiments, the compound, particle, or composition undergo uptake into cells. In some embodiments, the compound, particle, or composition undergo uptake into macrophage cells. In some embodiments, the compound, particle, or composition undergo uptake into dendritic cells. For example, in some embodiments, the compound, particle, or composition can be coated with dextran to target the macrophage cells, since macrophages have dextran receptors. In various embodiments, the method further comprises allowing the compound, particle, or composition to accumulate in a region of the biological tissue, wherein the targeting domain facilitated accumulation of the compound, particle, or composition in the region.

In various aspects, the compound, particle, or composition of the present invention can be used alone or in combination with a therapeutic agent to deliver a therapeutic agent payload to a target cell. Often, the therapeutic agent may be released based on the degradation of, e.g., a controlled release biodegradable matrix and/or polymer. However, it has been found that the compounds or particles of the present invention can also deliver their payload by hydrolysis disruption of the compounds or particles.

The dosage of the compound or particle will vary according to a number of factors, such as the administration route, the age, weight and species of the subject, but in general containing in the order of from 1 mol/kg to 1 mmol/kg bodyweight of the compound or particle.

Administration may be topical, parenteral (e.g., intravenously, intraarterially, intramuscularly, interstitially, subcutaneously, transdermally, or intrasternally), or into an externally voiding body cavity (e.g., the gastrointestinal tract, rectum, bladder, uterus, vagina, nose, ears or lungs), peritoneally, orally, intradermal, ocular, in an animate human or non-human (e.g., mammalian, reptilian or avian) body.

### Kits

The present invention also pertains to kits useful in the methods of the invention. Such kits comprise various combinations of components useful in any of the methods described elsewhere herein, including for example, materials for modulating function of cells using the particles of the invention, materials for modulating a metabolic inhibition of cells using the particles of the invention, materials for modulating an immunoresponse using the particles of the invention, materials for treating a disease or disorder using the particles of the invention, and instructional material. For example, in one embodiment, the kit comprises components useful for the modulation of cell function in a subject. In a further embodiment, the kit comprises components useful for the modulation of a metabolic inhibition in a cell. In a further embodiment, the kit comprises components useful for the modulation of an immunoresponse in a subject. In a further embodiment, the kit comprises components useful for the treatment of a disease or disorder in a subject.

### Embodiments

Embodiment 1 is a composition comprising a particle, wherein the particle comprises:
a) a nucleic acid molecule or a fragment or variant thereof comprising a nucleotide sequence that encodes at least one antigen or a fragment or variant thereof, and
b) a compound comprising the structure of Formula (I)

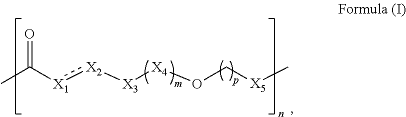

or a derivative or a pharmaceutical acceptable salt thereof,
wherein each occurrence of X₁ and X₂ is independently C═R₁, CR₂, or CR₃R₄;
each occurrence of X₃ and X₄ is independently C═R₁ or CR₃R₄;
each occurrence of X₅ is independently O, S, C═R₁, CR₃R₄, NR₂, PR₂, or P(═R₁)(R₂);
the bond between X₁ and X₂ is a single bond or a double bond;
wherein when the bond between X₁ and X₂ is a single bond, X₁ and X₂ are each independently C═R₁ or CR₃R₄, and when the bond between X₁ and X₂ is a double bond X₁ and X₂ are each CR₂;
each occurrence of R₁ is independently selected from the group consisting of O, NH and S;
each occurrence of R₂, R₃, and R₄ is independently selected from the group consisting of hydrogen, hydroxyl, carboxyl, alkyl, cycloalkyl, heterocycloalkyl, aryl, and heteroaryl;
each occurrence of m is independently an integer represented by 0, 1, 2, or 5;
each occurrence of p is independently an integer from 1 to 50; and
each occurrence of n is independently an integer from 1 to 1000.

Embodiment 2 is the composition of embodiment 1, wherein the compound comprising the structure of Formula (I) is a compound comprising the structure of Formula (II)

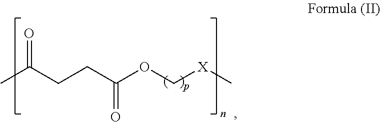

or a derivative or a pharmaceutical acceptable salt thereof,
wherein each occurrence of X is independently O, S, C═R₁, CR₃R₄, NR₂, PR₂, or P(═R₁)(R₂);
each occurrence of R₁ is independently selected from the group consisting of O, NH and S;
each occurrence of R₂, R₃, and R₄ is independently selected from the group consisting of hydrogen, hydroxyl, carboxyl, alkyl, cycloalkyl, heterocycloalkyl, aryl, and heteroaryl;
each occurrence of p is independently an integer from 1 to 15; and
each occurrence of n is independently an integer from 1 to 1000.

Embodiment 3 is the composition of embodiment 1, further comprising an adjuvant.

Embodiment 4 is the composition of embodiment 3, wherein the adjuvant is operably linked to the compound comprising the structure of Formula (I), the nucleic acid molecule, or both.

Embodiment 5 is a composition comprising a particle, wherein the particle comprises:
a) a nucleic acid molecule or a fragment or variant thereof comprising a nucleotide sequence that encodes at least one antigen or a fragment or variant thereof, and
b) a metabolite or a derivative or a polymer thereof.

Embodiment 6 is the composition of embodiment 5, further comprising an adjuvant.

Embodiment 7 is the composition of embodiment 6, wherein the adjuvant is operably linked to the metabolite or the derivative or the polymer thereof, the nucleic acid molecule, or both.

Embodiment 8 is the composition of embodiment 7, wherein the particle comprises a compound comprising the structure of

or a derivative or a pharmaceutical acceptable salt thereof,
or a derivative or a pharmaceutical acceptable salt thereof,
or a derivative or a pharmaceutical acceptable salt thereof, or
or a derivative or a pharmaceutical acceptable salt thereof,
wherein each occurrence of M is independently selected from the group consisting of: Ca, Mg, Na, K, Sr, Zn, Fe, Co, and Cu;
each occurrence of metabolite is independently a metabolite or derivative thereof;
each occurrence of n is independently an integer from 1 to 1000; and
each occurrence of p is independently an integer represented by 0 or 1.

Embodiment 9 is the composition of any one of embodiment 5-8, wherein the metabolite comprises at least one selected from the group consisting of phosphoenolpyruvate, phosphoenolpyruvic acid, glucono-lactone-6-phosphate, gluconate-6-phosphase, sedoheptulose-7-phosphate, ribulose, ribulose-5-phosphate (R5P), xylulose, xylulose-5-phosphate, fructose-1,6-biphosphate (F16BP), fructose 6 phosphate (F6P), fructose-2,6-biphosphate, glycerate-2-phosphate, glucerate-3-phosphate, malate, fumarate, succinate, isocitrate, citrate, cis-aconitate, malonyl-CoA, acetyl CoA, 3-methylbutyryl CoA, 2-methylbutyryl CoA, 3-ketoacyl CoA, 3-hydroxyacyl CoA, enoyl CoA, 3-ketoacyl functionalized metabolite, 3-hydroxyacyl functionalized metabolite, enoyl functionalized metabolite, fatty acids, caprylic acid, capric acid, lauric acid, myristic acid, palmitic acid, stearic acid, arachidic acid, behenic acid, lignoceric acid, cerotic acid, myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid, docosahexaenoic acid, and any combination thereof.

Embodiment 10 is the composition of any one of embodiments 3, 4, or 6-9, wherein the adjuvant comprises at least one selected from the group consisting of polyinosinic:polycytidylic acid (poly(I:C)) or analog thereof, muramyl dipeptide derivatives (MDP) or analog thereof, Alum and Emulsions, complete Freund's adjuvant (CFA), incomplete Freund's adjuvant (IFA), pattern recognition receptor (PRR) ligands, cyclic guanosine monophosphate-adenosine monophosphate (2′3′-cGAMP), bis-(3′-5′)-cyclic dimeric adenosine monophosphate (c-di-AMP), Rp,Rp-isomer of the 2′3′-bisphosphorothioate analog of 3′3′-cyclic adenosine monophosphate (2′3′-c-di-AM(PS)2 (Rp,Rp)), cyclic diguanylate monophosphate-stimulator of interferon genes (c-di-GMP STING)-based vaccine adjuvant, CL401, CL413, CL429, Flagellin, Imiquimod, lipopolysaccharide (LPS) from the gram-negative bacteria E. coli 0111:B4 (LPS-EB), monophosphoryl lipid A from Salmonella minnesota R595 lipopolysaccharide (MPLA-SM), synthetic monophosphoryl lipid A (MPLA), oligodeoxynucleotides (ODN) 1585, ODN 1826, ODN 2006, ODN 2395, Pam3CSK4, Resiquimod (R848), trehalose-6,6-dibehenate (TDB), and any combination thereof.

Embodiment 11 is the composition of any one of embodiments 1-10, wherein the nucleic acid molecule comprises a nucleic acid molecule derived from a cancer cell, a synthetic nucleic acid molecule, or a combination thereof.

Embodiment 12 is the composition of any one of embodiments 1-11, wherein the nucleic acid molecule comprises an isolated mRNA or a fragment or variant thereof derived from a B16F10 cell, YUMM cell, and/or 4T1 cell.

Embodiment 13 is the composition of embodiment 11 or 12, wherein the cancer cell is a human cancer cell or canine cancer cell.

Embodiment 14 is the composition of embodiment 11 or 12, wherein the cancer cell is a melanoma cell.

Embodiment 15 is the composition of any one of embodiments 1-14, wherein the antigen comprises a tumor antigen or a fragment or variant thereof.

Embodiment 16 is the composition of any one of embodiments 1-15, wherein the composition comprises calcium and phosphorous in a ratio of between about 2:about 1 to about 3:about 1.

Embodiment 17 is the composition of any one of embodiments 1-16, wherein the particle induces glycolysis in at least one cell of interest.

Embodiment 18 is the composition of embodiment 17, wherein the cell of interest is an immune cell.

Embodiment 19 is the composition of embodiment 18, wherein the immune cell is selected from the group consisting of a dendritic cell (DC), macrophage, T cell, natural killer cell, eosinophil, basophil, B cell, neutrophil, and any combination thereof.

Embodiment 20 is the composition of any one of embodiments 1-19, wherein the particle has an average diameter of between about 1 μm to about 100 μm.

Embodiment 21 is the composition of any one of embodiments 1-20, further comprising a therapeutic agent.

Embodiment 22 is the composition of embodiment 21, wherein the therapeutic agent comprises at least one metabolic inhibitor, metabolic pathway accelerator, peptide therapeutic, protein therapeutic, and/or enzyme to accelerate metabolism.

Embodiment 23 is the composition of embodiment 22, wherein the at least one metabolic inhibitor is a glycolytic inhibitor, pentose phosphate pathway inhibitor, oxidative phosphorylation (OXPHOS) inhibitor, electron transport chain (ETC) inhibitor, and/or polyamine pathway inhibitor.

Embodiment 24 is the composition of embodiment 23, wherein the at least one metabolic inhibitor comprises PFK15.

Embodiment 25 is the composition of embodiment 23 or 24, wherein the composition inhibits glycolysis in at least one cancer cell.

Embodiment 26 is the composition of any one of embodiments 1-25, wherein the composition is a vaccine.

Embodiment 27 is a method of inducing an immune response in a subject, wherein the method comprises administering to the subject a therapeutically effective amount of the composition of any one of embodiments 1-26.

Embodiment 28 is a method of inhibiting glycolysis in at least one cancer cell of interest and simultaneously inducing or rescuing glycolysis in at least one non-cancer cell of interest,
wherein the method comprises administering to the subject a therapeutically effective amount of the composition of any one of embodiments 23-25.

Embodiment 29 is a method of preventing or treating a metabolic inhibition of at least one cell in a subject in need thereof, wherein the method comprises administering a therapeutically effective amount of the composition comprising at least one particle, wherein the particle comprises:
a) a compound comprising at least one selected from the group consisting of:
   i) a metabolite or a derivative or a polymer thereof; and
   ii) the structure of Formula (I)

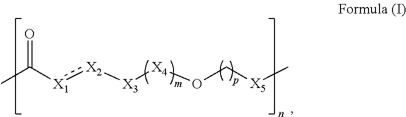

or a derivative or a pharmaceutical acceptable salt thereof,
wherein each occurrence of X₁ and X₂ is independently C═R₁, CR₂, or CR₃R₄;
each occurrence of X₃ and X₄ is independently C═R₁ or CR₃R₄;
each occurrence of X₅ is independently O, S, C═R₁, CR₃R₄, NR₂, PR₂, or P(═R₁)(R₂);
the bond between X₁ and X₂ is a single bond or a double bond;
wherein when the bond between X₁ and X₂ is a single bond, X₁ and X₂ are each independently C═R₁ or CR₃R₄, and when the bond between X₁ and X₂ is a double bond X₁ and X₂ are each CR₂;
each occurrence of R₁ is independently selected from the group consisting of O, NH and S;
each occurrence of R₂, R₃, and R₄ is independently selected from the group consisting of hydrogen, hydroxyl, carboxyl, alkyl, cycloalkyl, heterocycloalkyl, aryl, and heteroaryl;
each occurrence of m is independently an integer represented by 0, 1, 2, or 5;
each occurrence of p is independently an integer from 1 to 50; and
each occurrence of n is independently an integer from 1 to 1000; and
b) at least one selected from the group consisting of
   i) a nucleic acid molecule or a fragment or variant thereof comprising a nucleotide sequence that encodes at least one antigen or a fragment or variant thereof, and
   ii) an antigen or a fragment or variant thereof.

Embodiment 30 is a method of preventing or treating a disease or disorder in a subject in need thereof, wherein the method comprises administering a therapeutically effective amount of the composition comprising at least one metabolic inhibitor, metabolic pathway accelerator, peptide therapeutic, protein therapeutic, enzyme to accelerate metabolism, or any combination thereof and at least one particle,
wherein the particle comprises:
a) a compound comprising at least one selected from the group consisting of:
   i) a metabolite or a derivative or a polymer thereof, and
   ii) the structure of Formula (I)

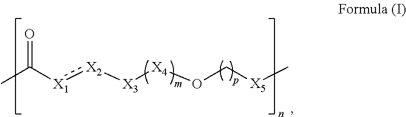

or a derivative or a pharmaceutical acceptable salt thereof,
wherein each occurrence of X₁ and X₂ is independently C═R₁, CR₂, or CR₃R₄;
each occurrence of X₃ and X₄ is independently C═R₁ or CR₃R₄;
each occurrence of X₅ is independently O, S, C═R₁, CR₃R₄, NR₂, PR₂, or P(═R₁)(R₂);
the bond between X₁ and X₂ is a single bond or a double bond;
wherein when the bond between X₁ and X₂ is a single bond, X₁ and X₂ are each independently C═R₁ or CR₃R₄, and when the bond between X₁ and X₂ is a double bond X₁ and X₂ are each CR₂;
each occurrence of R₁ is independently selected from the group consisting of O, NH and S;
each occurrence of R₂, R₃, and R₄ is independently selected from the group consisting of hydrogen, hydroxyl, carboxyl, alkyl, cycloalkyl, heterocycloalkyl, aryl, and heteroaryl;
each occurrence of m is independently an integer represented by 0, 1, 2, or 5;
each occurrence of p is independently an integer from 1 to 50; and
each occurrence of n is independently an integer from 1 to 1000; and
b) at least one selected from the group consisting of
   i) a nucleic acid molecule or a fragment or variant thereof comprising a nucleotide sequence that encodes an antigen or a fragment or variant thereof, and
   ii) an antigen or a fragment or variant thereof.

Embodiment 31 is the method of embodiment 29 or 30, wherein the metabolite comprises at least one selected from the group consisting of phosphoenolpyruvate, phosphoenolpyruvic acid, glucono-lactone-6-phosphate, gluconate-6-phosphase, sedoheptulose-7-phosphate, ribulose, ribulose-5-phosphate (R5P), xylulose, xylulose-5-phosphate, fructose-1,6-biphosphate (F16BP), fructose 6 phosphate (F6P), fructose-2,6-biphosphate, glycerate-2-phosphate, glucerate-3-phosphate, malate, fumarate, succinate, isocitrate, citrate, cis-aconitate, malonyl-CoA, acetyl CoA, 3-methylbutyryl CoA, 2-methylbutyryl CoA, 3-ketoacyl CoA, 3-hydroxyacyl CoA, enoyl CoA, 3-ketoacyl functionalized metabolite, 3-hydroxyacyl functionalized metabolite, enoyl functionalized metabolite, fatty acids, caprylic acid, capric acid, lauric acid, myristic acid, palmitic acid, stearic acid, arachidic acid, behenic acid, lignoceric acid, cerotic acid, myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid, docosahexaenoic acid, and any combination thereof.

Embodiment 32 is the method of any one of embodiments 29-31, wherein the composition induces or rescues at least one selected from the group consisting of: a glycolysis, tricarboxylic acid (TCA) cycle, polyamine pathway, pentose phosphate pathway (PPP), activation of the at least one cell, extracellular acidification rate (ECAR), oxygen consumption rate (OCR), mitochondrial respiration, release of a metabolite, pro-inflammatory response, BRAF inhibitor, tyrosine kinase inhibitor, extracellular signal-regulated kinase (ERK inhibitor), MEK inhibitor, and cancer cell suppression.

Embodiment 33 is the method of embodiment 30, wherein the composition:
a) decreases the level of at least one immune suppressive cell;
b) increases the level of at least one selected from the group consisting of: a T cell, type 1 CD8+ T cell (Tc1), type 2 CD8+ T cell (Tc2), IL-17-producing CD8+ T cell (Tc17), T helper cell (Th), Th1, Th17, and effector T cell (Teff); or
c) both a) and b).

Embodiment 34 is the method of embodiment 30, wherein the composition induces or rescues glycolysis in at least one cell of interest and inhibits glycolysis in a cancer cell.

Embodiment 35 is the method of embodiment 34, wherein the at least one cell of interest is an immune cell.

Embodiment 36 is the method of embodiment 34, wherein the composition reduces or inhibits a tumor growth; reduces a cancer cell proliferation; or a combination thereof.

Embodiment 37 is the method of embodiment 30, wherein the disease or disorder is cancer.

Embodiment 38 is the method of any one of embodiments 27-37, wherein the method is an adoptive T cell therapy.

Embodiment 39 is a method comprising administering the composition of any one of embodiments 1-26 to a cell, wherein the cell phagocytes the particle.

Embodiment 40 is the method of embodiment 39, wherein the cell is an immune cell.

Embodiment 41 is the method of embodiment 40, wherein the immune cell is selected from the group consisting of a dendritic cell (DC), macrophage, T cell, natural killer cell, eosinophil, basophil, B cell, neutrophil, and any combination thereof.

Embodiment 42 is a method comprising administering the composition of any one of embodiments 1-26 to a subject in need thereof, wherein the subject has cancer.

Embodiment 43 is the method of embodiment 42, wherein the composition thereof is administered directly to the subject.

Embodiment 44 is the method of embodiment 42, wherein the method comprises the steps of:
a) administering the composition to a cell, wherein the cell phagocytes the particle; and
b) administering the cell comprising the phagocyted particle to the subject.

Embodiment 45 is the method of any one of embodiments 39-44, wherein the method further comprises administering a glycolytic inhibitor to the subject.

### EXPERIMENTAL EXAMPLES

The present invention is further described in detail by reference to the following experimental examples. These examples are provided for purposes of illustration only, and are not intended to be limiting unless otherwise specified. Thus, the present invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples and therefore, specifically point out the exemplary embodiments of the present invention, and are not to be construed as limiting in any way the remainder of the disclosure.

### Example 1: Rescue from Glycolysis Inhibition of Dendritic Cells Restored Function and Generated Robust Cancer Immunotherapy in Mice

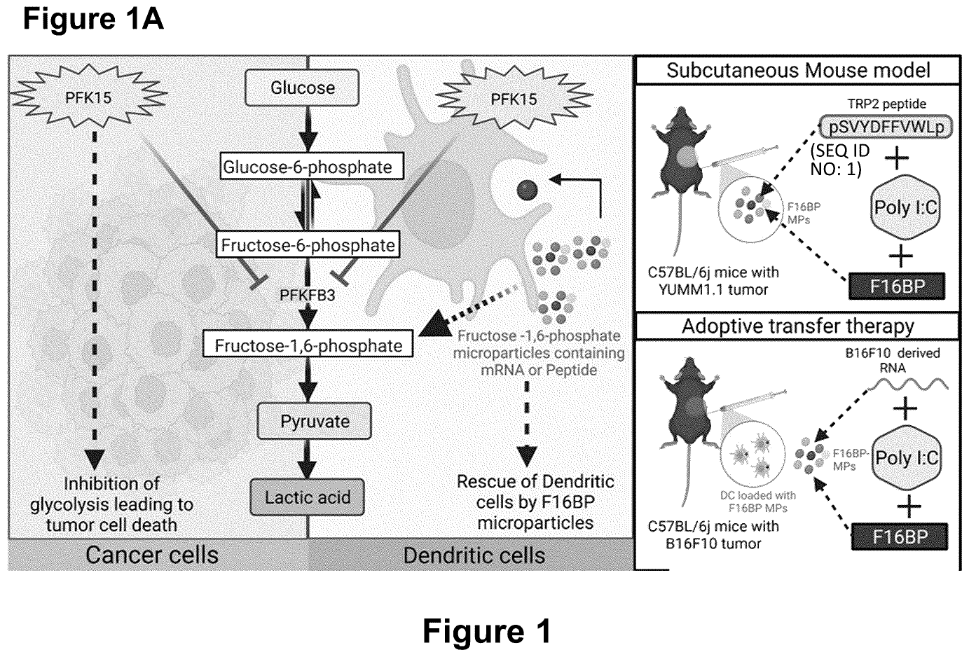

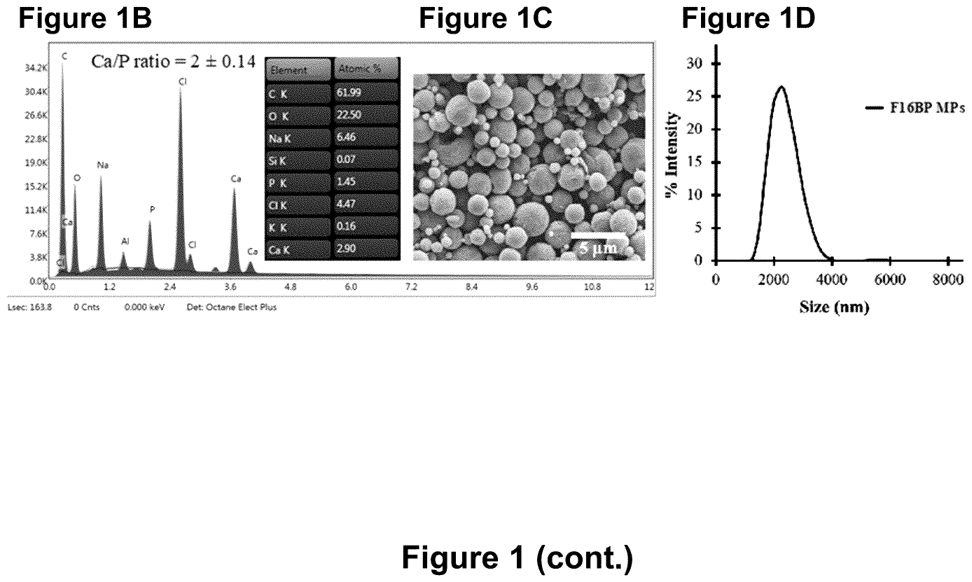

Immunometabolism reprogramming is an emerging and exciting field for generating effective cancer immunotherapy. Glycolysis reprogramming in DCs can be performed by overexpressing glycolytic enzymes or glucose receptors, however these require genetic manipulation of immune cells, which can be challenging. Another approach to accelerate glycolysis is to provide glycolytic metabolites exclusively to DCs, which then satisfy the energy needs of these cells, while blocking glycolysis of cancer cells. However, these metabolites are small molecules that get consumed or diffuse away after administration in vivo. Therefore, there is a need to generate technologies that can provide sustained release of metabolites to DCs. Notably, DCs are phagocytes, and thus phagocytosable particles that release glycolytic metabolites in a sustained manner can be effective in allowing glycolysis to occur in these cells. In fact, to simultaneously block glycolysis in cancer cells and allow DC-based immunotherapy, glycolytic inhibitors are provided systemically, while providing the metabolite-based particles to DCs (FIG. 1A).

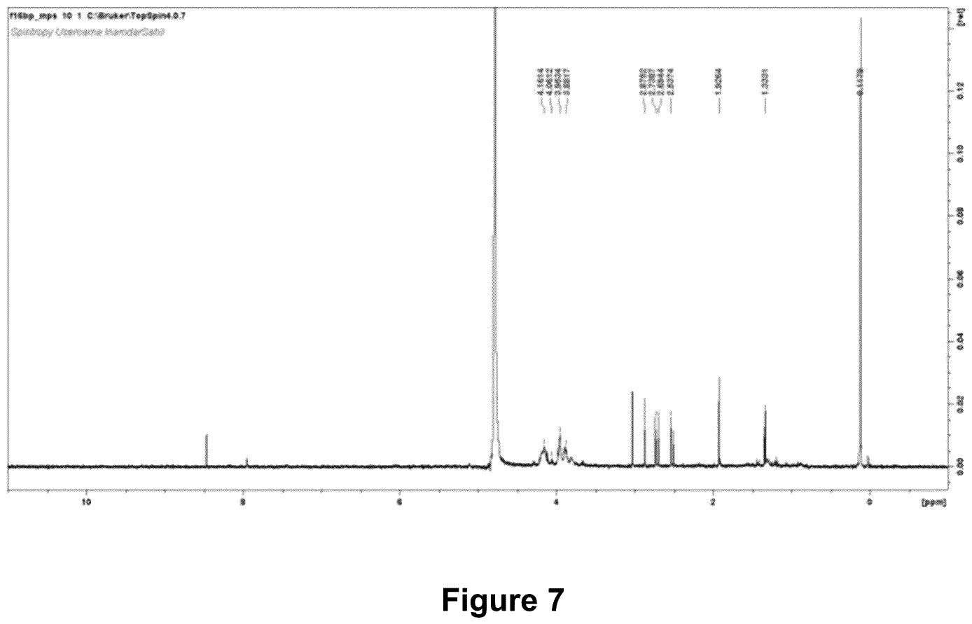

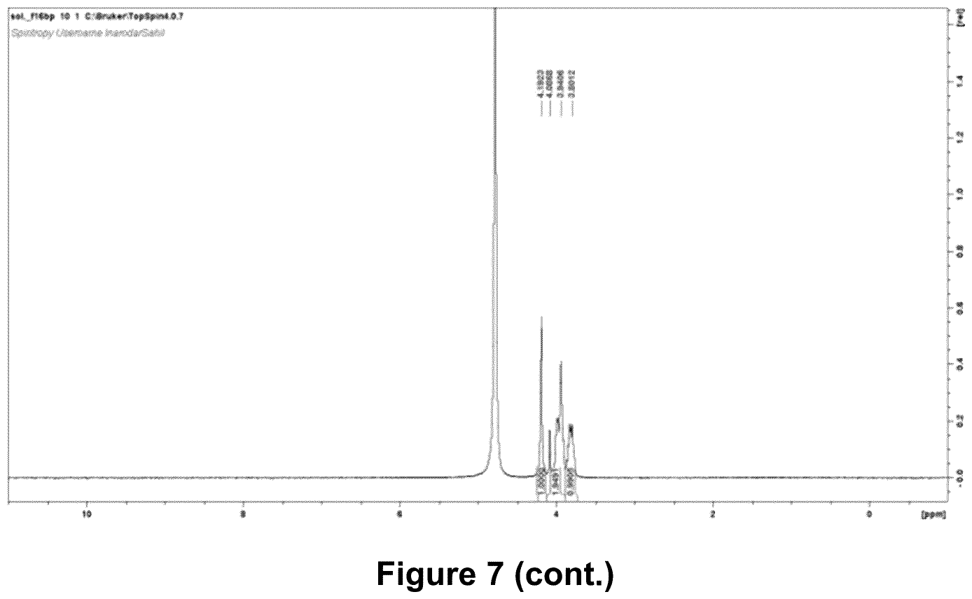

To demonstrate the strength of such a strategy, PFK15 (which blocks 6-phosphofructo-2-kinase fructose-2,6-biphosphatase 3-PFKFB3) was chosen to block glycolysis, as it is one of the rate limiting steps in glycolysis. Moreover, fructose-1,6-biphosphate (F16BP), which is generated by phosphofructokinase (PFK) in a step downstream of PFKFB3, was chosen as the metabolite to generate phagocytosable particles. Novel F16BP based microparticles (MPs) were generated using calcium-phosphate chemistry. Dynamic light scattering demonstrated that the size of these particles was 2.3±0.4 μm (FIG. 1B) and scanning electron micrographs showed that these particles had smooth spherical morphology (FIG. 1C). Using ¹H NMR and EDX-mapping, it was determined that the F16BP was incorporated within these microparticles (FIG. 1C and FIG. 7) and the particles had 2±0.14 of Calcium to Phosphorous (Ca:P) ratio (FIG. 1C).

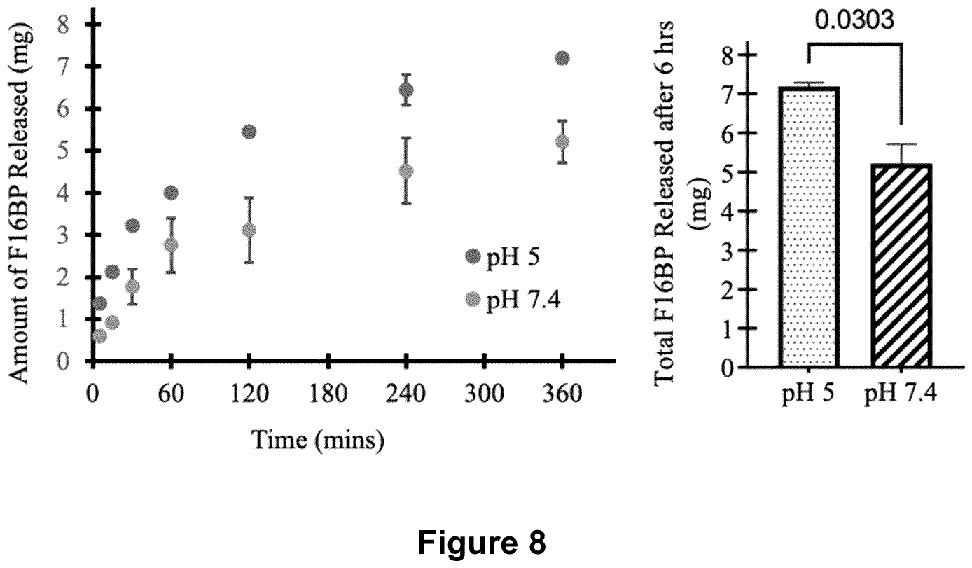

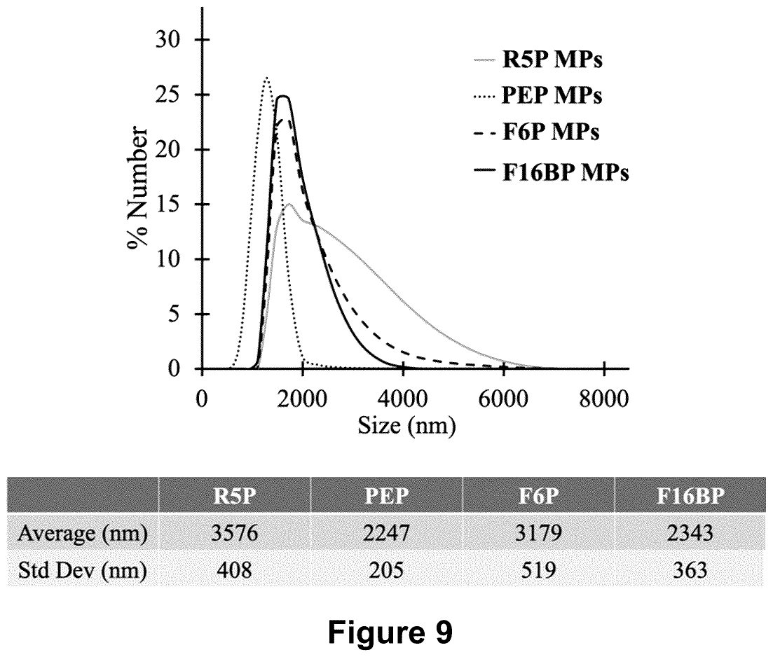

To test if F16BP MPs could release F16BP, release kinetics in phosphate buffered saline was performed. It was observed that F16BP MPs released F16BP for 6 hours in a sustained manner (FIG. 8). These data demonstrated that the MPs generated to contain F16BP, were in the phagocytosable range of DCs and released F16BP to allow glycolysis to move forward. In addition to F16BP, particles of other control metabolites, such as ribulose 5 phosphate (R5P), phosphoenolpyruvic acid (PEP), and fructose 6 phosphate (F6P) were also generated (FIG. 9).

To test if these particles can be phagocytosed by DCs, confocal imaging was performed. Specifically, F16BP MPs were generated with FITC intercalated within the particles. Bone marrow derived DCs (DCs) were then incubated with these particles for 60 minutes, and stained for actin and nuclei, and fluorescent imaging was performed. Cytochalasin D in the presence of F16BP-FITC MPs were used as control. It was observed that DCs were able to associate with the particles effectively (FIG. 10).

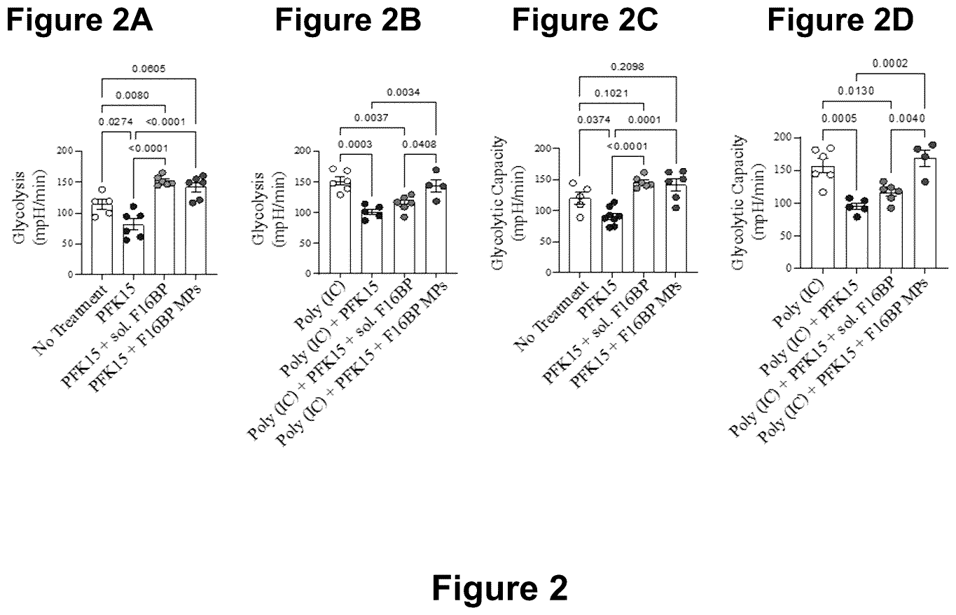

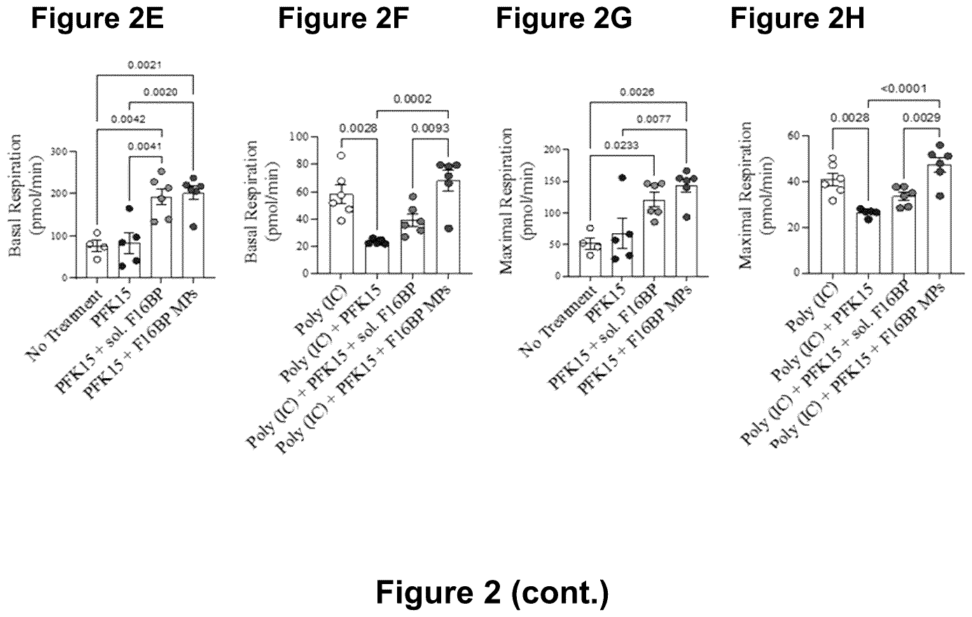

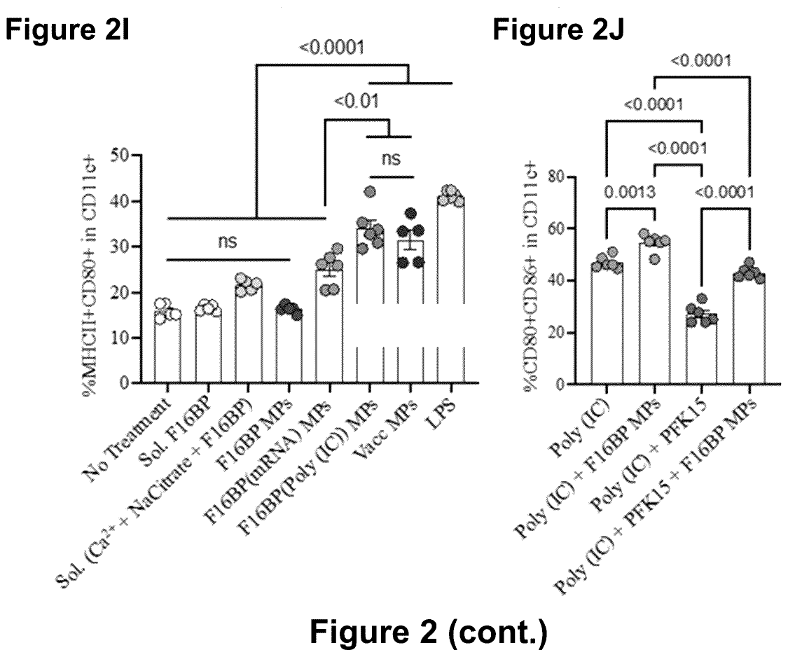

Next, the ability of the F16BP MPs to rescue glycolysis in DCs in the presence of glycolytic inhibitor PFK15 was evaluated using extracellular flux assays. DCs were cultured with either PFK15, F16BP MPs, soluble fructose-6-phosphate (F6P—upstream of PFKFB3) or PFK15+F16BP MPs, or individual components of the F16BP MPs for 2 hr, and extracellular acidification rate (ECAR—rate of glycolysis) measurements were obtained. It was observed that PFK15 brought the glycolysis and glycolytic capacity (y-axis—ECAR) lower than no treatment control (FIG. 2A through FIG. 2D and FIG. 11). Importantly, glycolysis and glycolytic capacity were significantly higher in F16BP MPs even in the presence of the PFK15, as compared to the PFK15 alone control (FIG. 2A through FIG. 2D). Also, in the presence of poly(IC) (an activating agent for DCs), PFK15 decreased the glycolysis and the glycolytic capacity, and the F16BP MPs were able to rescue this decrease even in the presence of PFK15 (FIG. 2E through FIG. 2H). Overall, these data indicated that the phagocytosable F16BP MPs were able to rescue glycolysis in DCs even in the presence of glycolytic inhibitor PFK15, and thus can be utilized for immunotherapies where glycolysis pathway of cancer cells is targeted. This is important since under conditions of inflammation, DCs need to perform enhanced levels of glycolysis to support the inflammatory protein production.

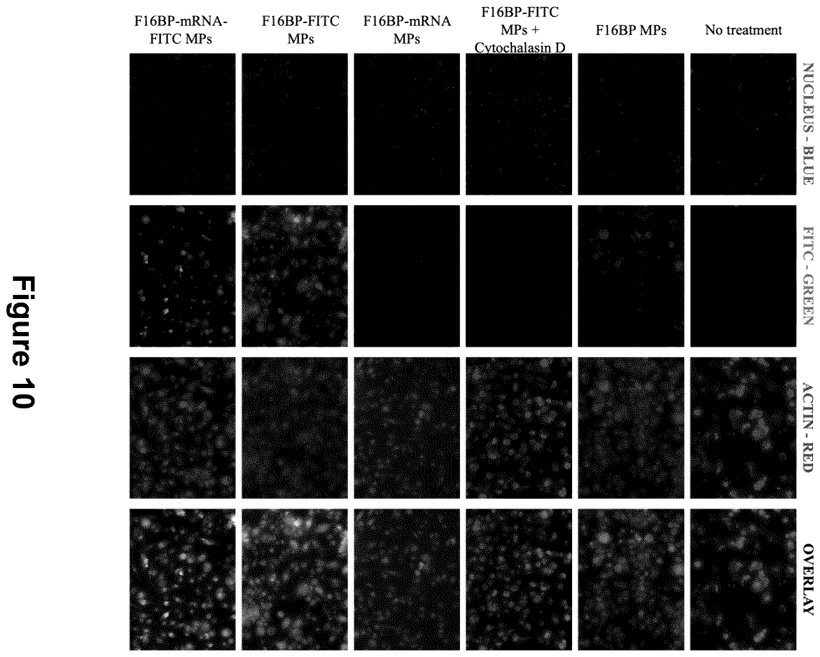

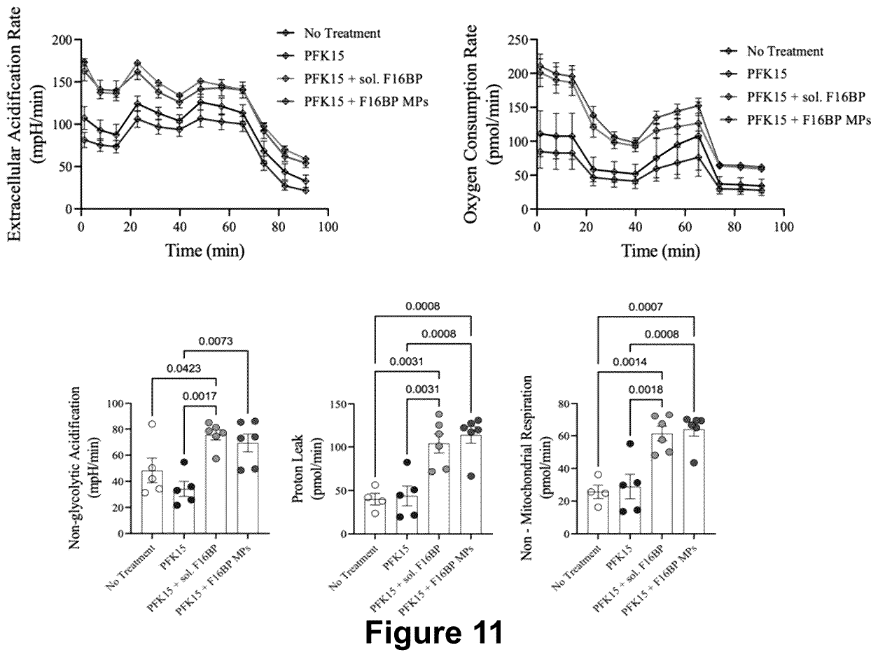

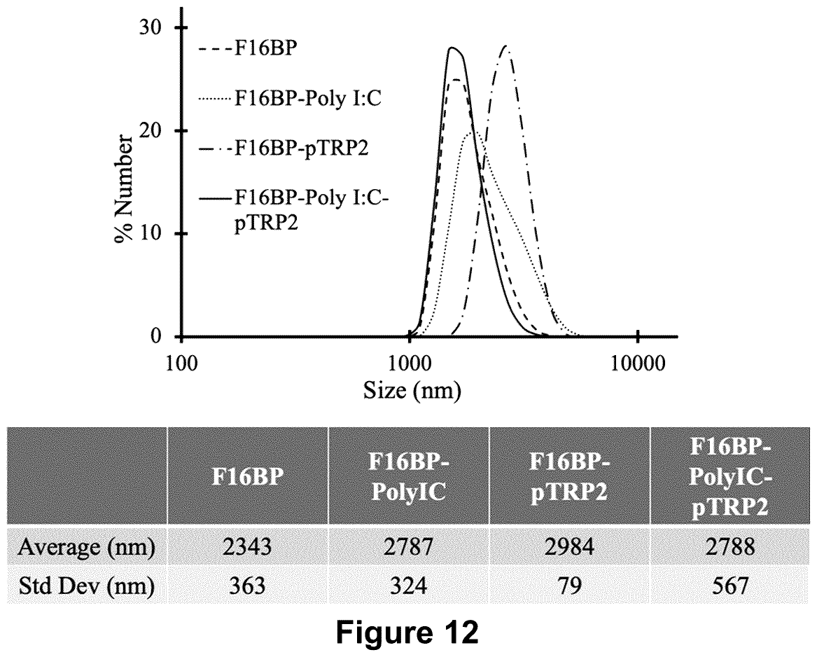

To test if F16BP MPs can modulate the function of DCs, mRNA-based and peptide-based vaccine F16BP MPs were formulated. These vaccines were generated by intercalating di-phosphorylated melanoma peptide antigen Tyrosine related protein 2 (pTRP2), and poly(IC) and mRNA derived from melanoma cells, which contain several phosphate groups in their backbone (FIG. 12). Since pTRP2p, and poly(IC) have phosphate groups, these molecules can be incorporated into the F16BP MPs using the calcium-phosphate chemistry. The amount of pTRp2, and poly(IC) incorporated within the F16BP MPs was determined to be 78±3.4, and 50.9±7.9 μg, respectively. Next, F16BP MPs intercalated with poly(IC) and pTRP2 were incubated with DCs overnight, and flow cytometry was utilized to test if these particles could activate DCs (FIG. 2I and FIG. 2J). It was observed that the vaccine particles induced significantly higher frequency of MHCII+CD86+ in CD11c+ DCs as compared to the individual component controls of the MPs (FIG. 2I). Additionally, it was observed that PFK15 was able to decrease the activation of DCs (CD80+CD86+ in CD11c+) even in the presence of poly(IC), and F16BP MPs were able to rescue the activation of DCs even in the presence of PFK15 (FIG. 2J). These data indicate that the F16BP based vaccine MPs rescue DC activation even in the presence of PFK15, which is important if in vivo cancer vaccine responses need to be generated in the presence of glycolysis inhibition.

To test if the immunometabolism modulating approach of rescuing glycolysis in the presence of glycolytic inhibitors and generating cancer vaccine immunotherapies, highly aggressive forms of melanoma mouse models were chosen. Specifically, vaccine MPs were injected subcutaneously in mice containing YUMM1.1 (murine BRAF^{v600e} mutation similar to humans) melanoma tumors, and their ability to reduce tumor growth and modulate innate and adaptive immune responses was tested.

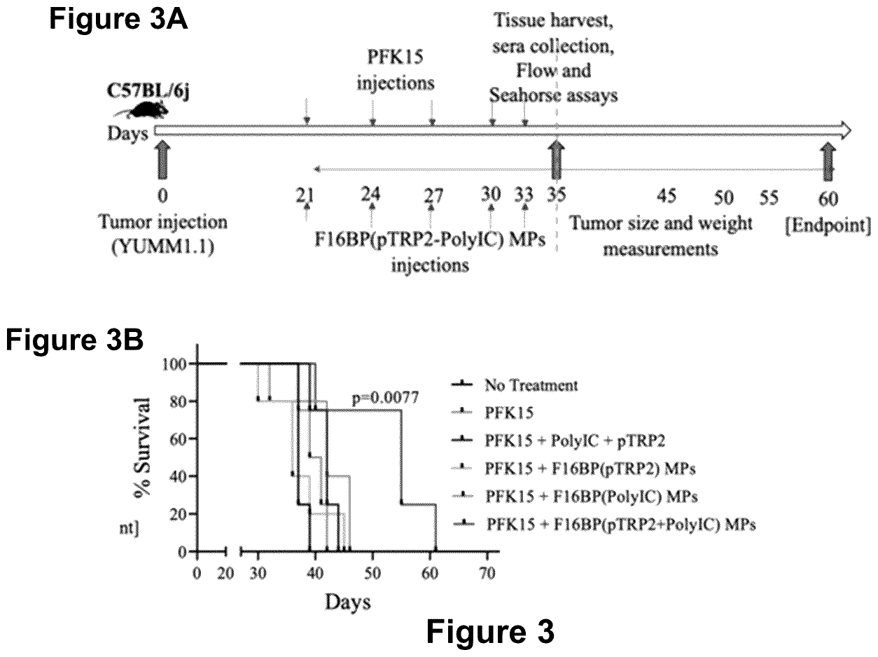

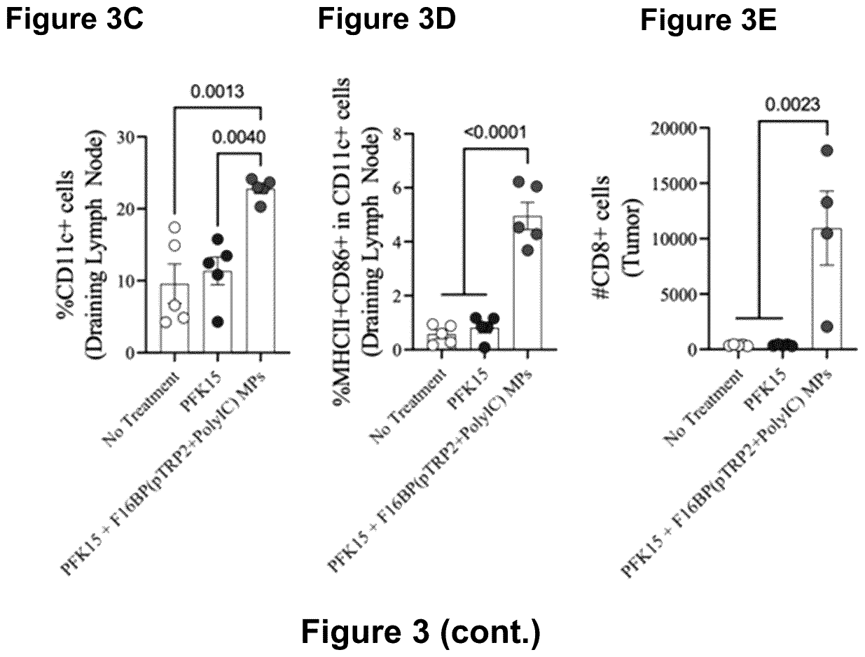

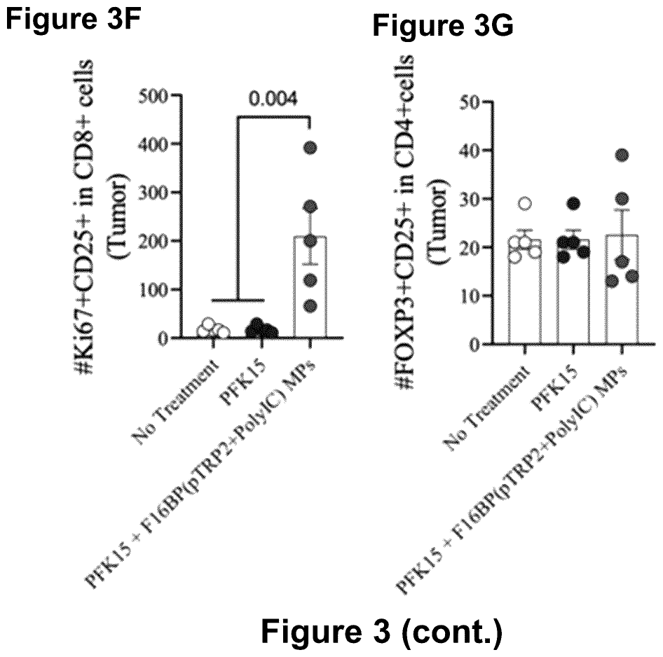

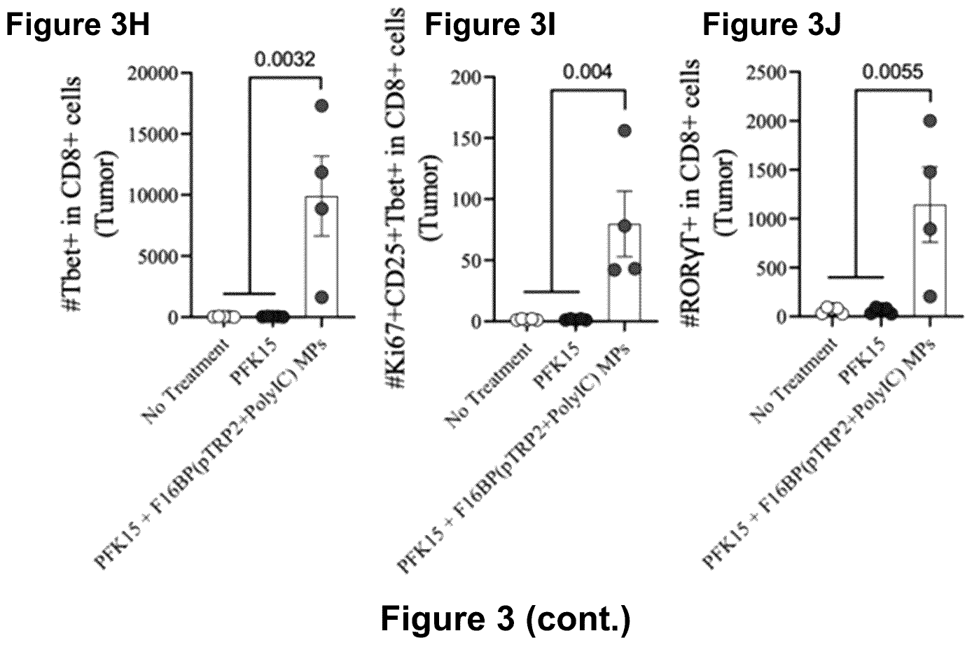

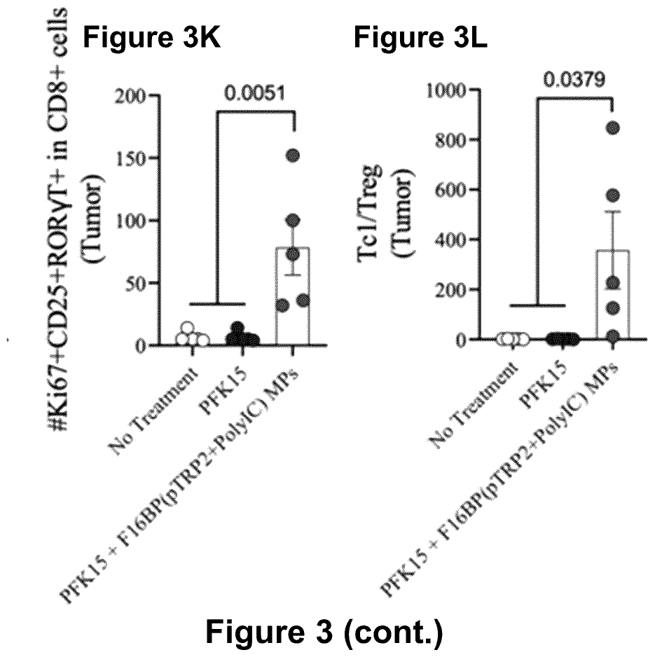

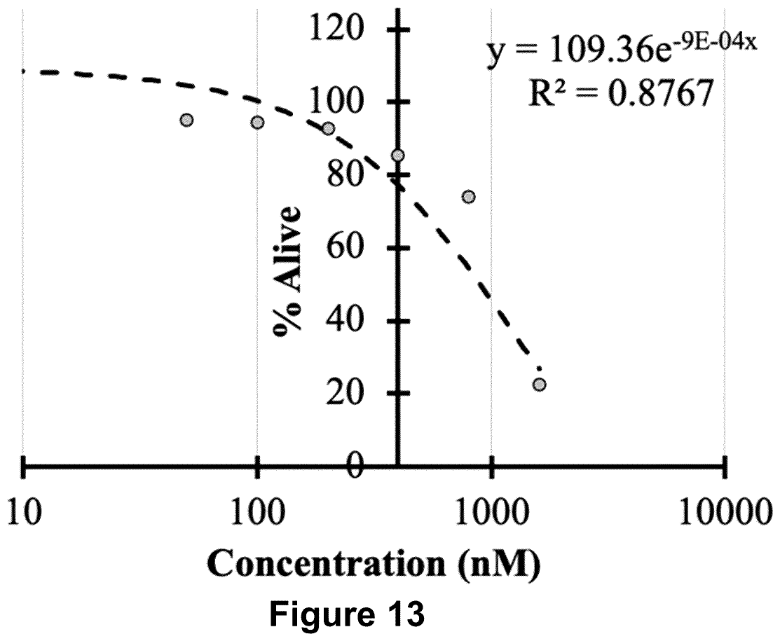

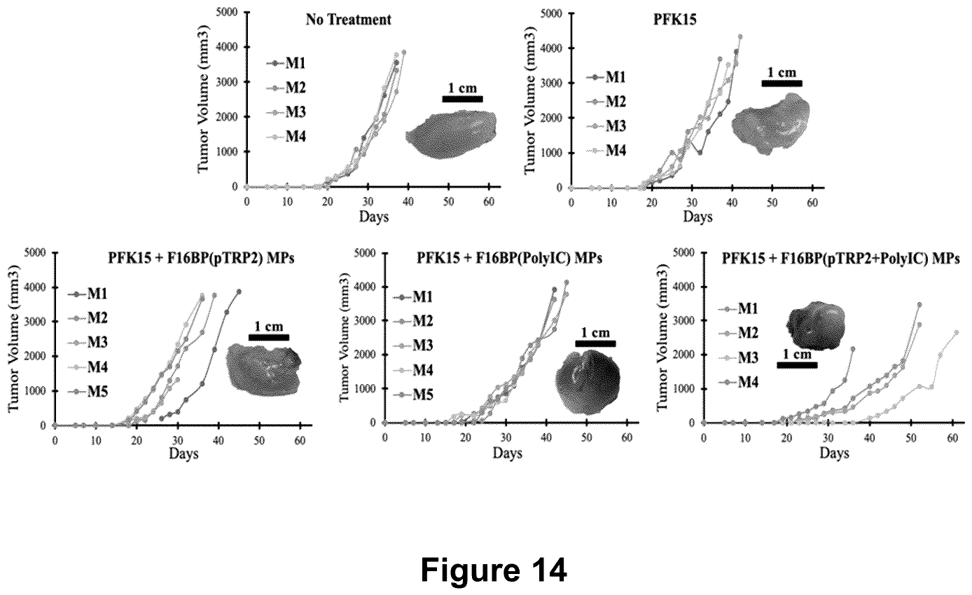

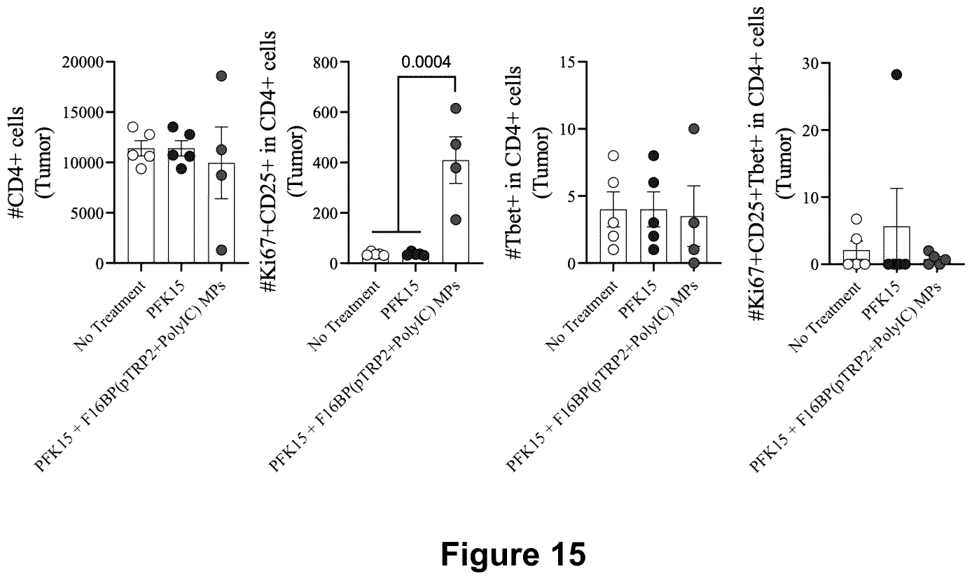

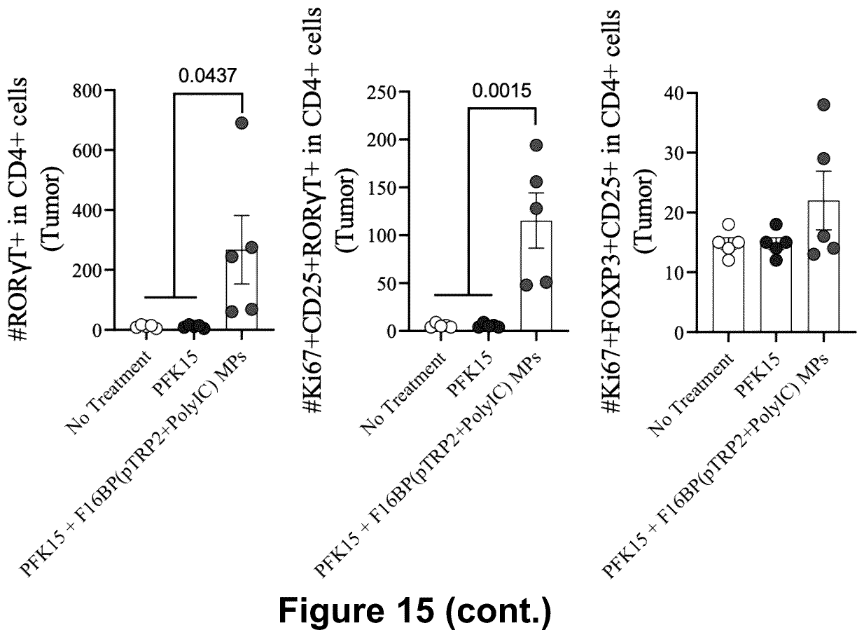

In this melanoma model, 0.75×10⁶ YUMM1.1 cells were injected subcutaneously in C57BL/6j immunocompetent mice, and PFK15 was injected every alternate day for the duration of the study. Moreover, F16BP(pTRP2+poly(IC)) were injected subcutaneously on the same days as the PFK15 injections (FIG. 3A). In vitro it was determined that PFK15 was effective in preventing proliferation of YUMM1.1 cancer cells (FIG. 13). It was observed that the treatment group of PFK15+vaccine MPs led to significantly increased survival of mice (day 60—endpoint) and slower tumor growth as compared to all the different controls (FIG. 3B and FIG. 14). This increase in survival in the vaccine MPs was associated with increase in DC population and activated DC population in the draining inguinal lymph nodes on day 35, which indicated that the vaccine MPs were able to modulate the innate immune responses (FIG. 3C and FIG. 3D). Within the tumor, no significant differences were observed in the number of CD4+ T cells of different treatment groups; however, there was a significant increase in the proliferating and activated CD4+ cells in mice treated with vaccine MPs as compared to the other treatment groups (FIG. 15). Furthermore, no significant differences were observed in the number of T helper type 1 (Th1) and activated and proliferating Th1 cells; significant increase in T helper type 17 (Th17) and activated and proliferating Th17 cells were observed in mice treated with vaccine MPs as compared to other treatment groups (FIG. 15). Moreover, within the tumor, there was a significant increase in the number of CD8+ T cells and proliferating and activated CD8+ T cells in mice treated with vaccine MPs as compared to other treatment groups indicating the decrease in tumor growth kinetics in mice treated with vaccine MPs responses (FIG. 3E and FIG. 3F). No significant differences were observed in regulatory T cells in the different treatment groups (FIG. 3G and FIG. 14). Additionally, there was a significant increase in the number of Tc1, Tc17 and proliferating and activated Tc1 and Tc17 cells in mice treated with vaccine MPs as compared to other treatment groups (FIG. 3H through FIG. 3K). Finally, a significant increase in the Tc1/Treg ratio was observed in mice treated with vaccine MPs as compared to other treatment groups (FIG. 3L). These data demonstrated that the vaccine MPs were able to generate robust pro-inflammatory adaptive immune responses in the tumor. Overall, these data demonstrated that the vaccine MPs that deliver F16BP and can rescue DCs, were able to generate robust immune responses against aggressive form of melanoma tumors

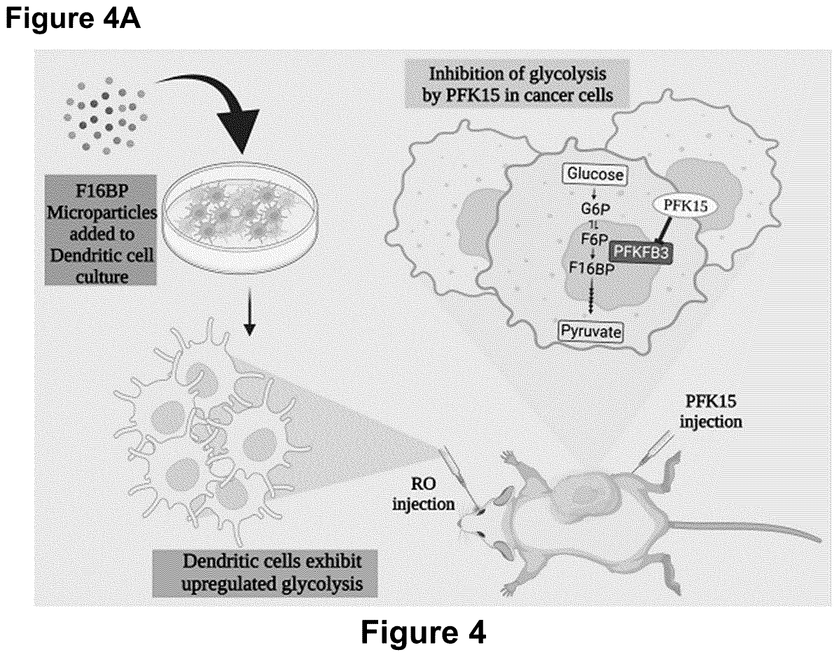

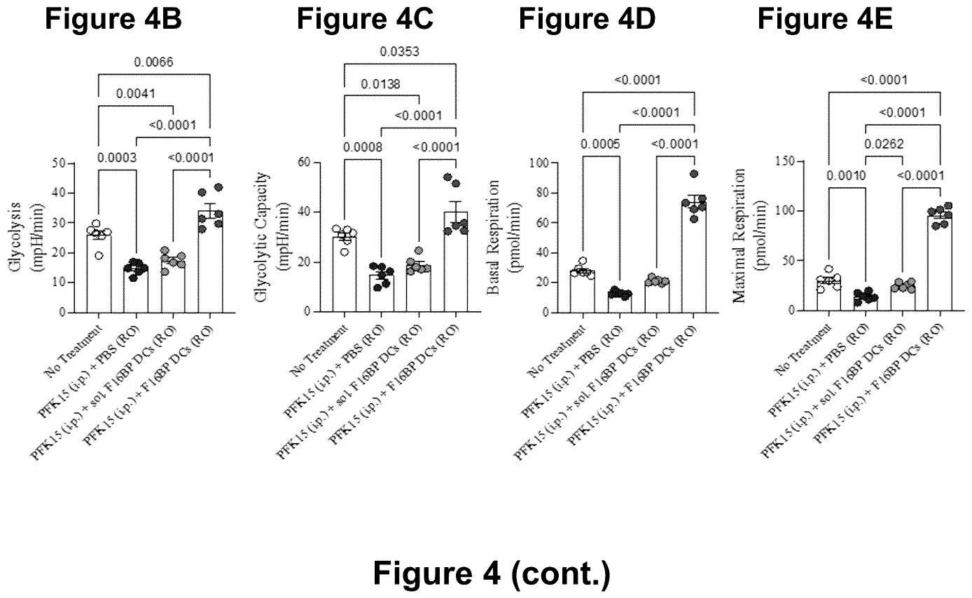

To test the versatility of the F16BP-PFK15 system, another aggressive B16F10 melanoma model was chosen (FIG. 4A). In this model, DCs were loaded with F16BP MPs intercalated with mRNA isolated from B16F10 cancer cells, and poly(IC) (Vacc DCs) and adoptively transferred in mice containing B16F10 tumors. This cellular therapy's ability to modulate adaptive immune responses, reduced tumor growth, and improve survival were measured.

First, to test if the F16BP MPs (without poly(IC) or mRNA) can rescue DCs in vivo, F16BP MPs were loaded into DCs by incubating these particles with DCs for 2 hr. Next, these DCs were adoptively transferred intravenously in mice and PFK15 was injected intraperitoneally. DCs were isolated from spleen and extracellular flux assays were performed on these cells. It was observed that the F16BP MPs were able to rescue glycolysis and glycolytic capacity of DCs as observed by the increased ECAR values in the presence of PFK15 as compared to the controls (FIG. 4B and FIG. 4C). Moreover, F16BP MPs were also able to rescue basal and maximal respiration of DCs in the presence of PFK15, as compared to the controls (FIG. 4D and FIG. 4E). This data demonstrated that these particles not only rescued glycolysis and mitochondrial respiration in these cells in vitro but also in vivo, and thus are able to generate functional immunotherapeutic responses.

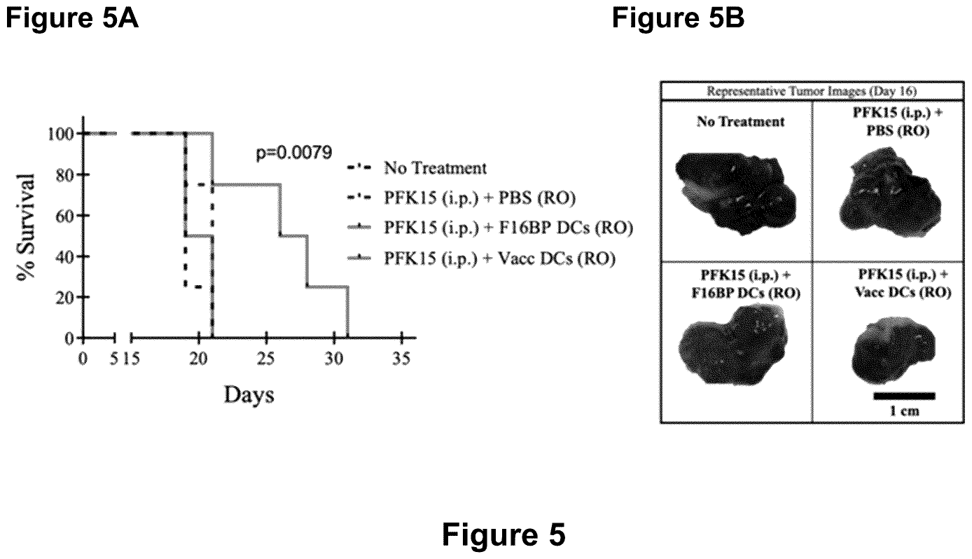

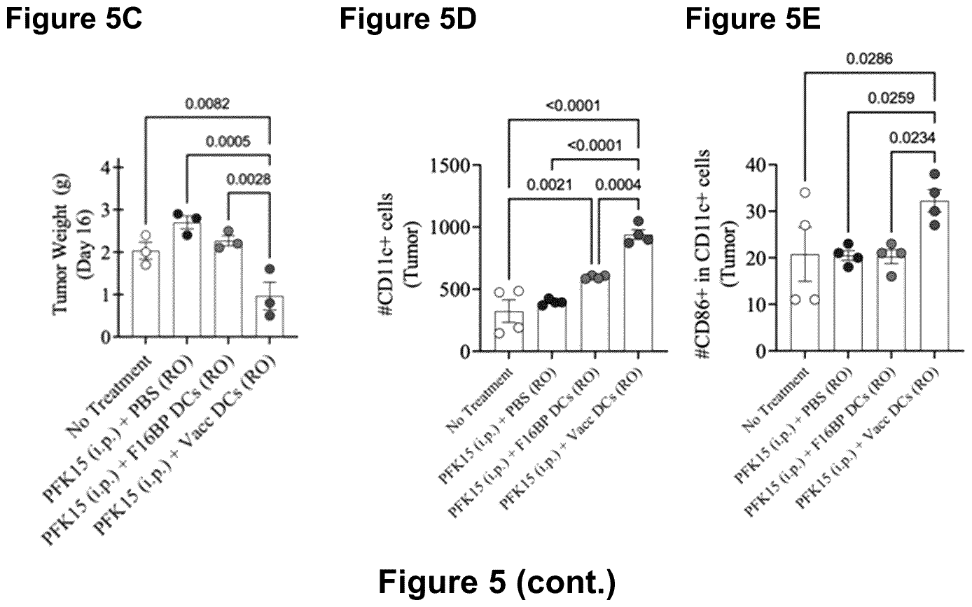

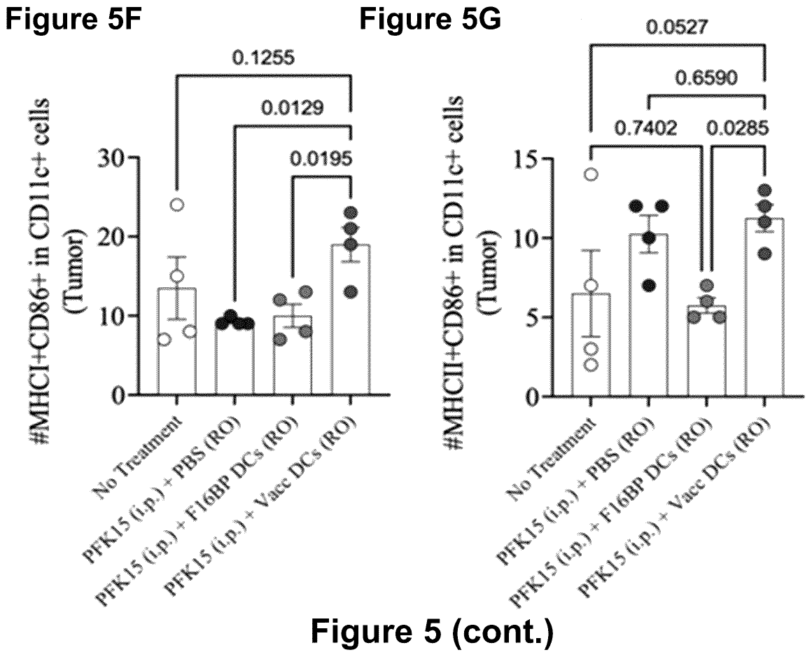

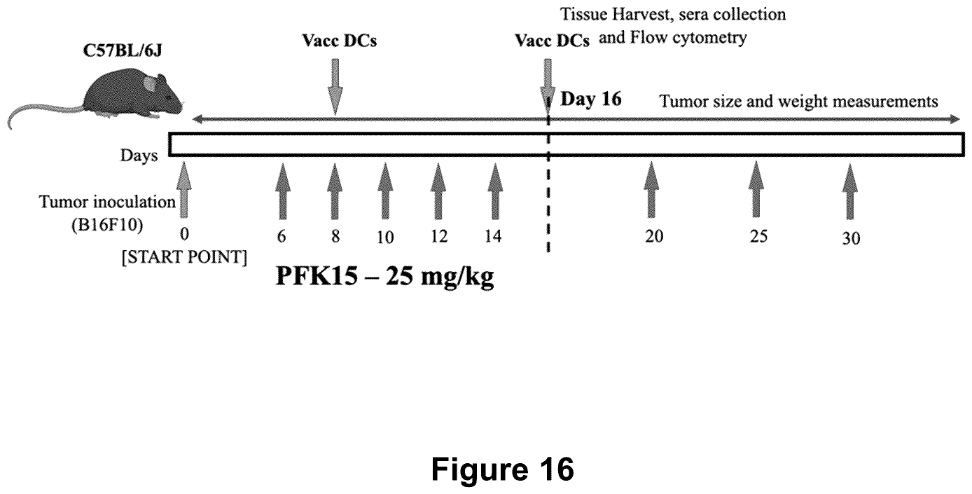

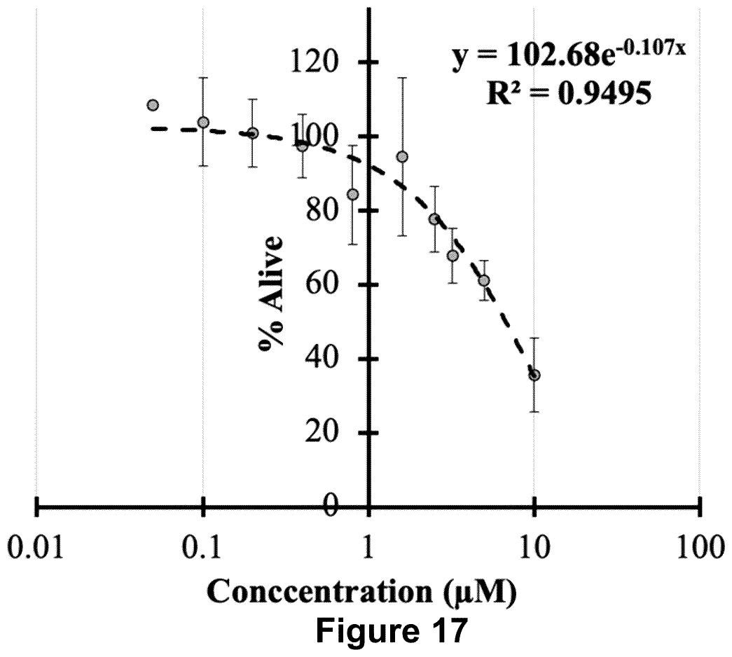

To test if the metabolic rescue in DCs can be applied to adoptive cell therapy, adoptive transfer of DC-vaccines was tested in mice (FIG. 16). First, in vitro it was determined that PFK15 was effective in preventing proliferation of B16F10 cancer cells (FIG. 17). Specifically, 0.75×10⁶ B16F10 cells were injected in C57BL/6j immunocompetent mice, and PFK15 was injected every alternate day for the duration of the study (FIG. 16). Moreover, ex vivo DCs loaded with F16BP(mRNA+poly(IC)) (Vacc DCs) or DCs loaded with F16BP MPs were adoptively transferred using retro-orbital injections (RO) on days 6 and 19 (FIG. 15). It was observed that the survival of mice receiving Vacc DCs+PFK15 increased dramatically as compared to all the controls, and the tumor grew slower in mice receiving Vacc DCs as compared to the controls (FIG. 5A and FIG. 17). This data was further corroborated by tumor weights and images that were obtained at half-point of the study at day 16 (FIG. 5B and FIG. 5C). These data indicated that after just two injections of Vacc DCs there was a robust response against the tumor, which then reduced the tumor growth in mice.

To test if the Vacc DCs could modulate the immune responses after their administration, on day 16, spleen, lymph nodes, and tumors were isolated from different conditions and stained for the activation profile of DCs, and T cells. It was observed that the total number of DCs and activated DCs in tumor for Vacc DCs was significantly higher as compared to all the controls (FIG. 5D and FIG. 5E). Moreover, Vacc DCs also upregulated number of DCs that were MHCI+CD86+CD11c+ in the tumor as compared to other treatment groups, suggesting the Vacc DCs support the activation of DCs in vivo (FIG. 5F). Also, Vacc DCs also upregulated number of DCs that were MHCII+CD86+CD11c+ in the tumor as compared to adoptively transferred F16BP DCs, suggesting the Vacc DCs skew toward MHCII associated responses in vivo (FIG. 5G). These data indicated that glycolysis are needed for potential infiltration of the DCs in the spleen and the tumor, and F16BP MIPs provided the ability to DCs for glycolysis to occur.

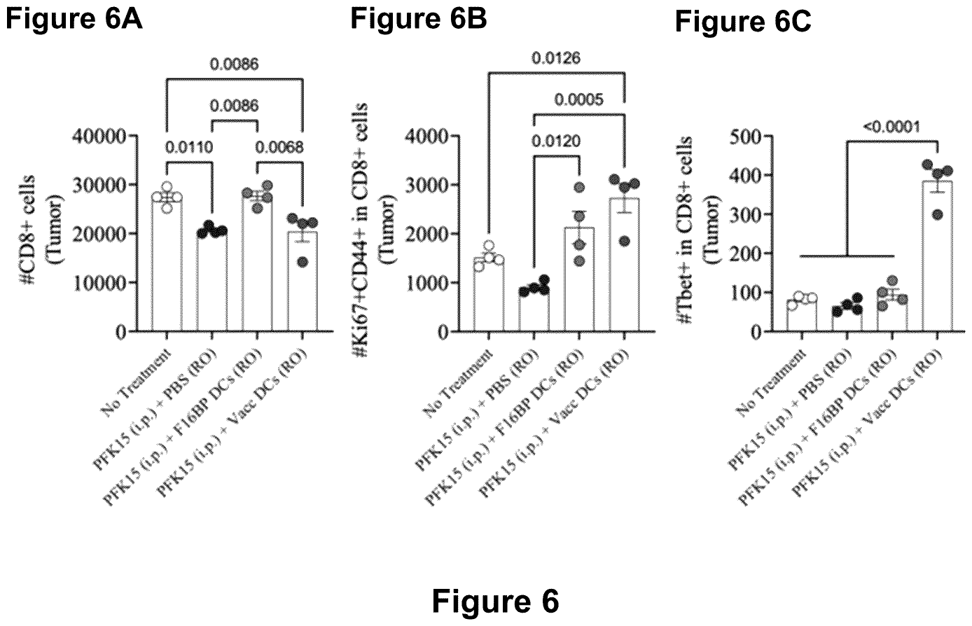

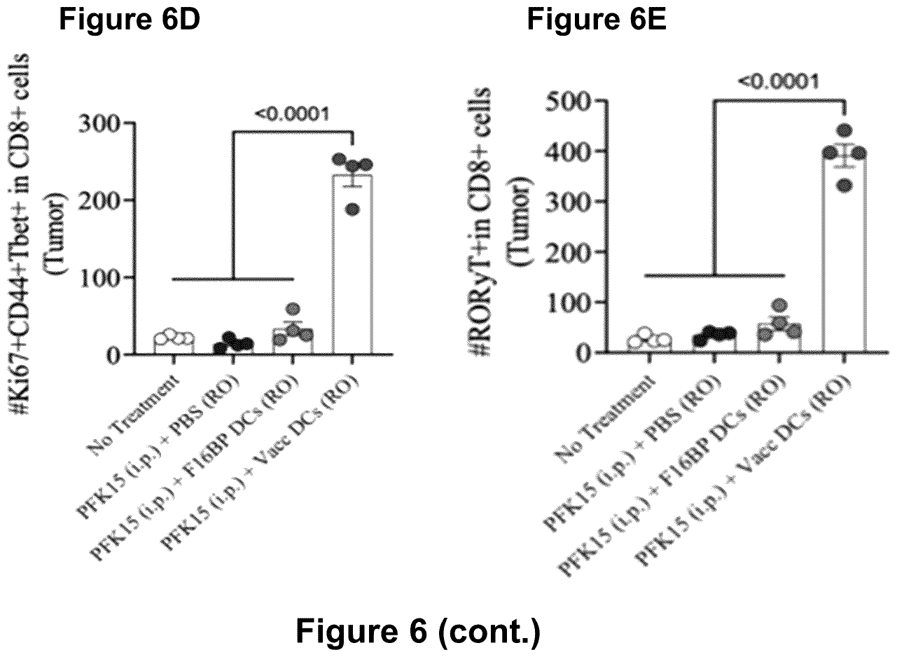

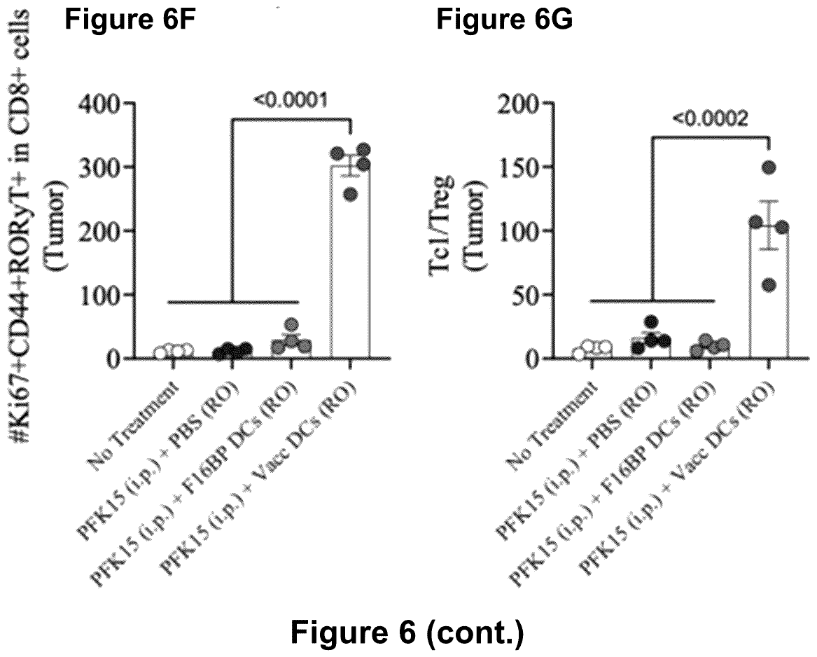

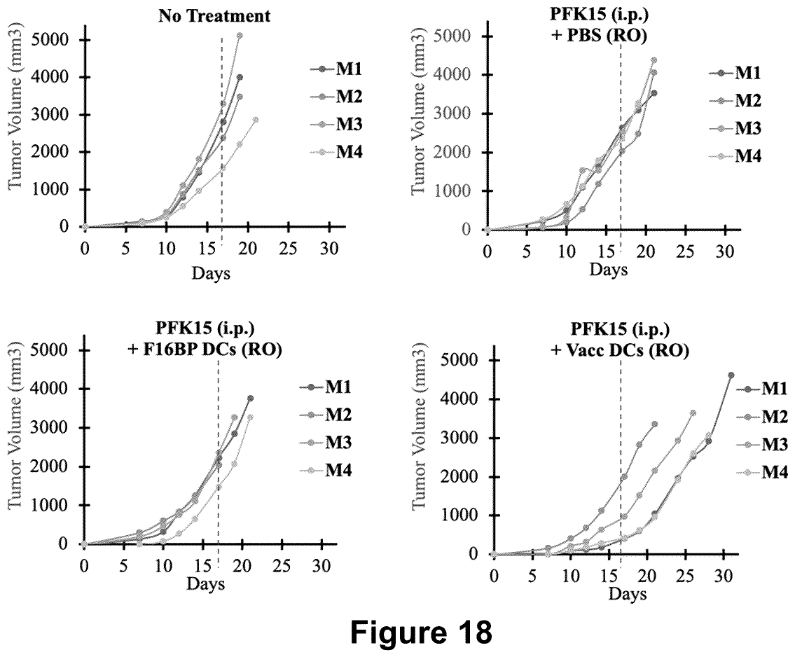

The adaptive T cell responses in the tumor were skewed toward pro-inflammatory responses (FIG. 6A through FIG. 6G and FIG. 18). It was observed that PFK15 treatment alone significantly decreased the total number of CD8+ T cells in the tumor, and that DCs loaded with F16BP, were able to bring this number equal to the no treatment control (FIG. 6A). Within this CD8+ T cell numbers, it was observed that, there was 2-3-fold increase in activated and proliferating (Ki67+CD44+ in CD8+) T cells in the tumor in Vacc DC group as compared to the control groups (FIG. 6B). Furthermore, there was 4-fold significant increase in cytotoxic Tbet+ in CD8+ T cells (Tc1) in the Vacc DCs group as compared to all the other groups (FIG. 6C). Moreover, the number of activated and proliferating Tc1 cells was >10-fold higher in Vacc DCs group as compared to all the other treatment conditions (FIG. 6D). Similarly, the number of Tc17 cells (RORγt+ in CD8+), and activated and proliferating Tc17 (RORγt+Ki67+CD44+ in CD8+), which are emerging as an important pro-inflammatory cell types that induce cancer cell death, were also >10-fold significantly higher than the controls in the tumors (FIG. 6E and FIG. 6F). In addition to the Tc populations, Vacc DCs also led to decrease in the numbers of T helper cells (Th), activated and proliferating Th cells, Th1 (Tbet+ in CD4+), Th2 (GATA3+ in CD4+), activated and proliferating Th1 (Tbet+CD44+Ki67+ in CD4+), and activated and proliferating Th2 cells (GATA3+CD44+Ki67+ in CD4+) numbers in the Vacc DC group (FIG. 18). There was a significant decrease in the number of Treg (CD25+Foxp3+ in CD4+) within the tumor in mice treated with Vacc DCs as compared to untreated mice (FIG. 18). Moreover, there was a significant increase in the Th17 (RORγt+ in CD4+) and activated and proliferating Th17 cells (RORγt+Ki67+CD44+ in CD4+) within the tumor in the Vacc DC treated mice as compared to all the other groups (FIG. 18). Furthermore, the ratio of Tc1 to Treg cells was 5-10 fold higher in the tumor of the Vacc DC treated mice as compared to the controls (FIG. 6G). These data indicated that the Vacc DCs were able to induce a robust adaptive immune response against the tumors, which were primarily driven by increases in Tc1 and Tc17 cell populations.

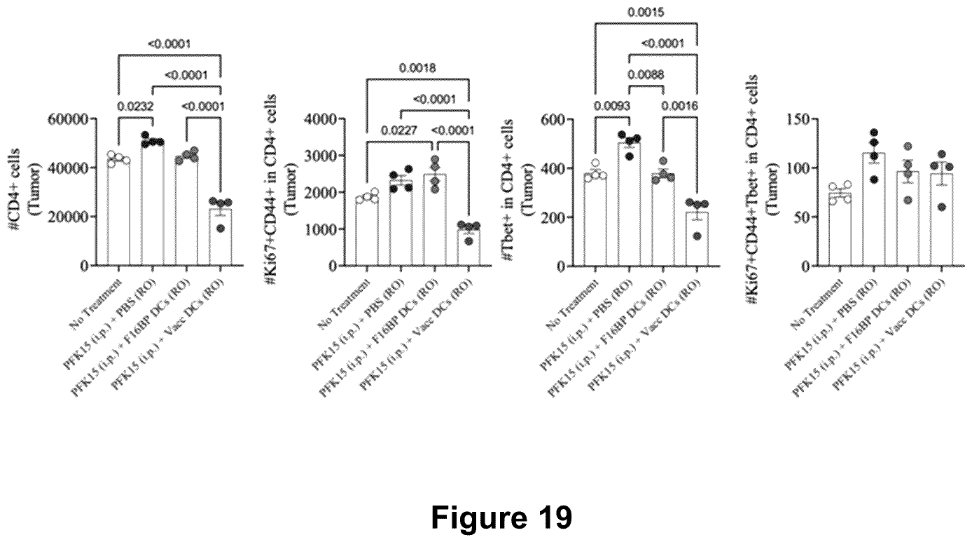

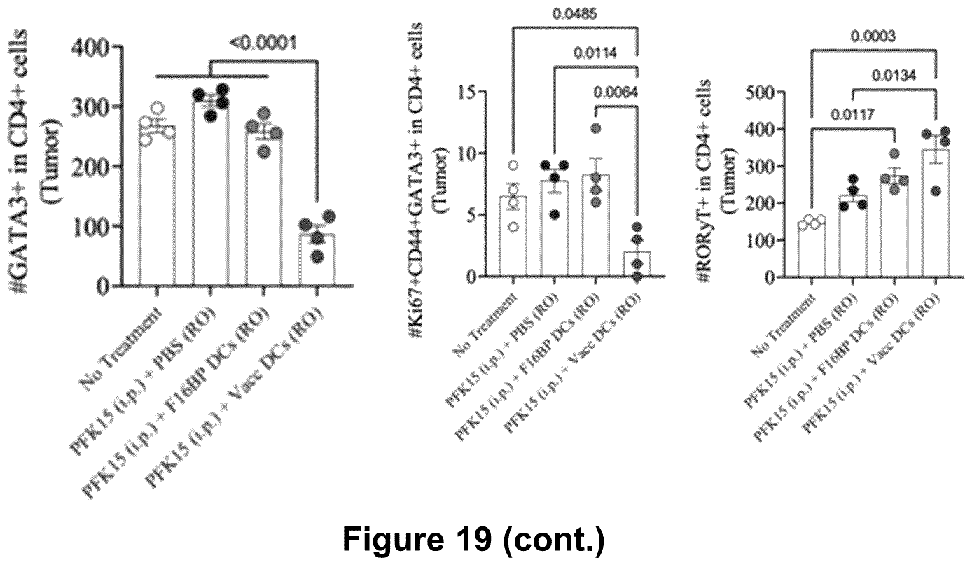

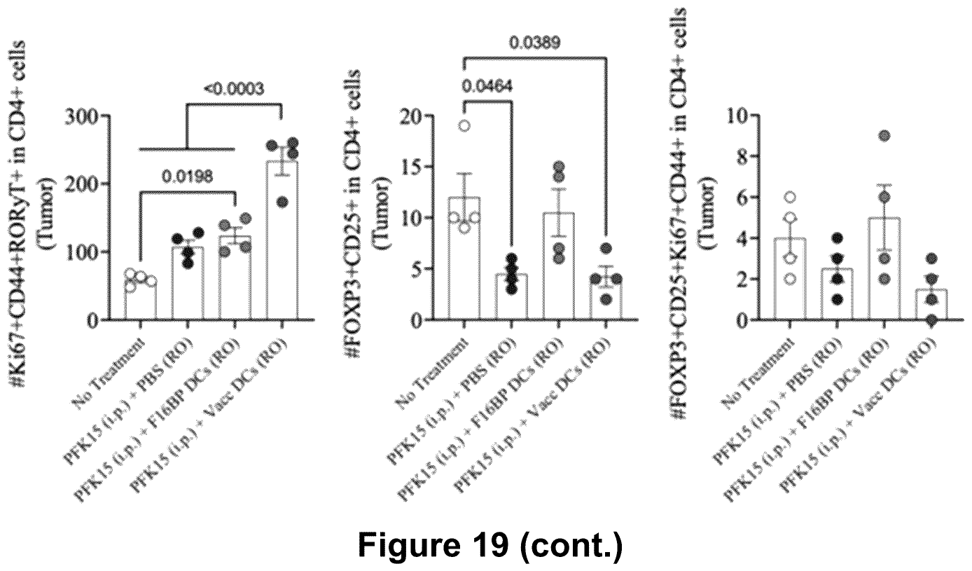

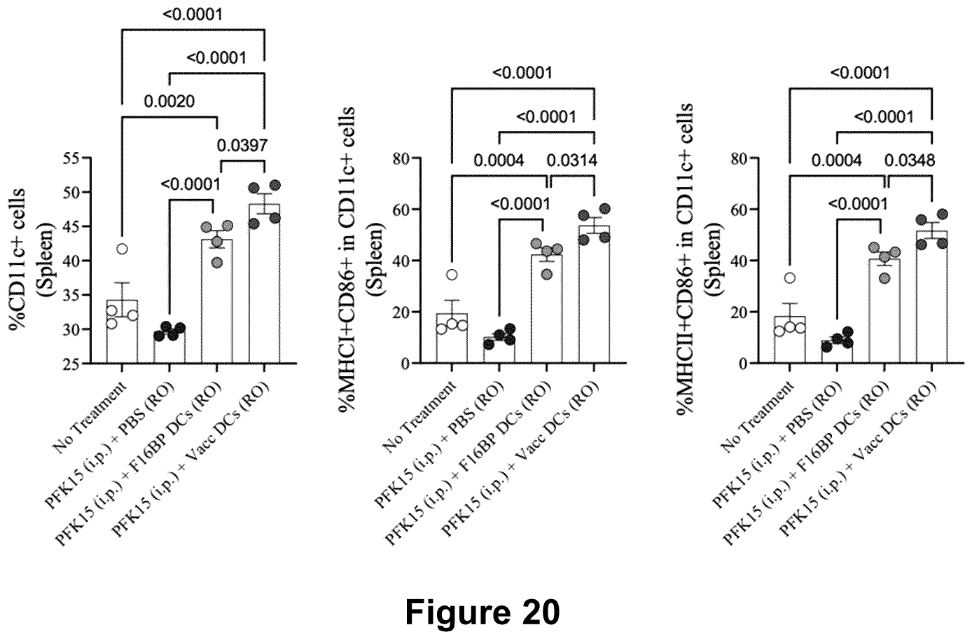

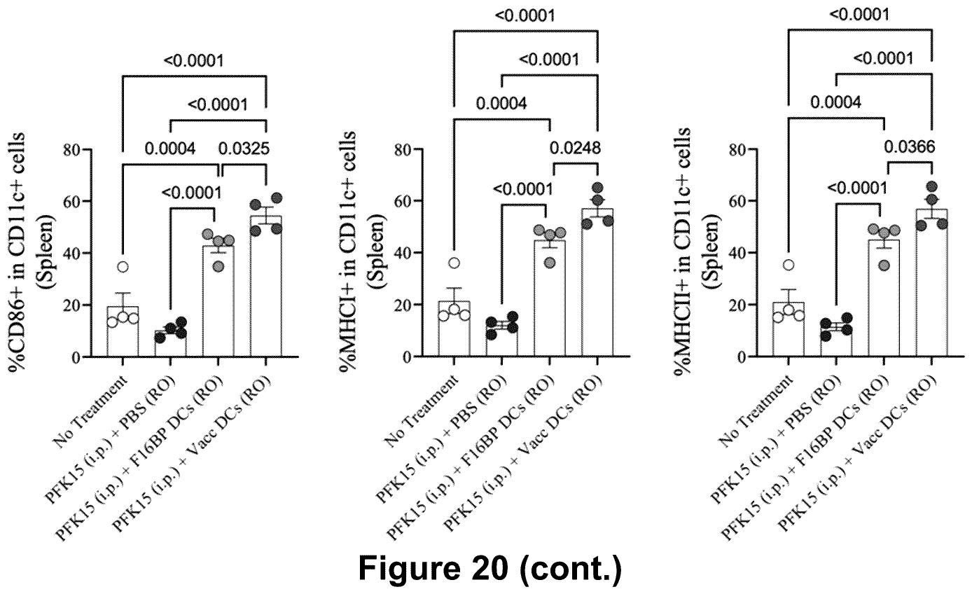

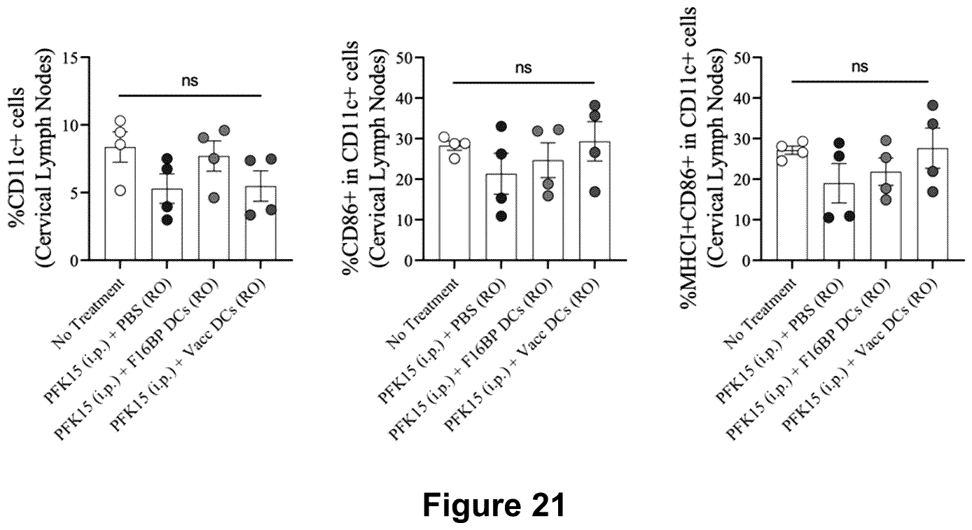

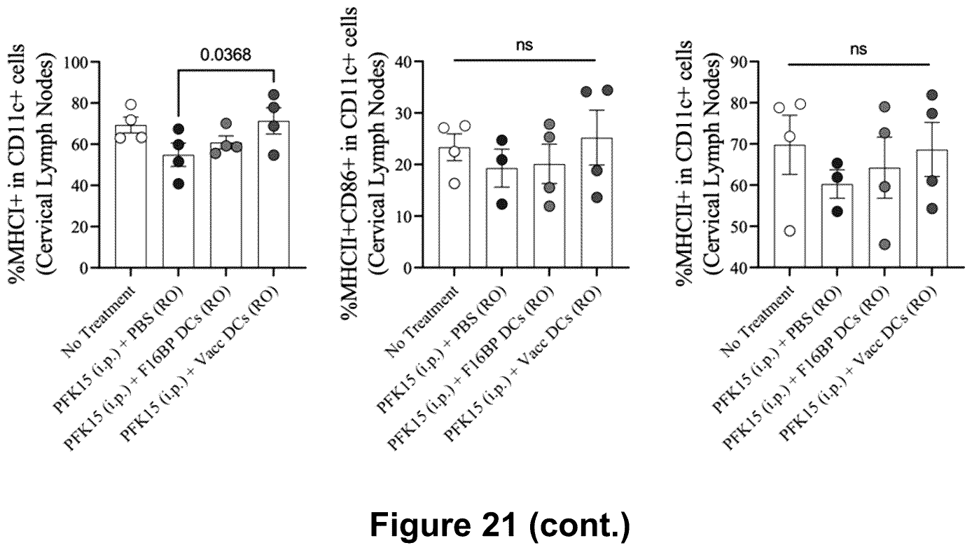

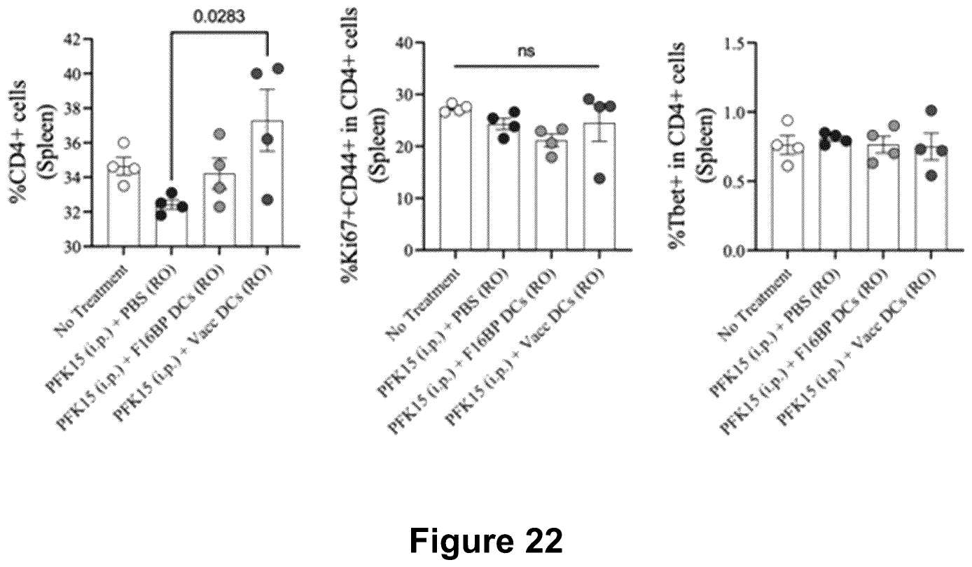

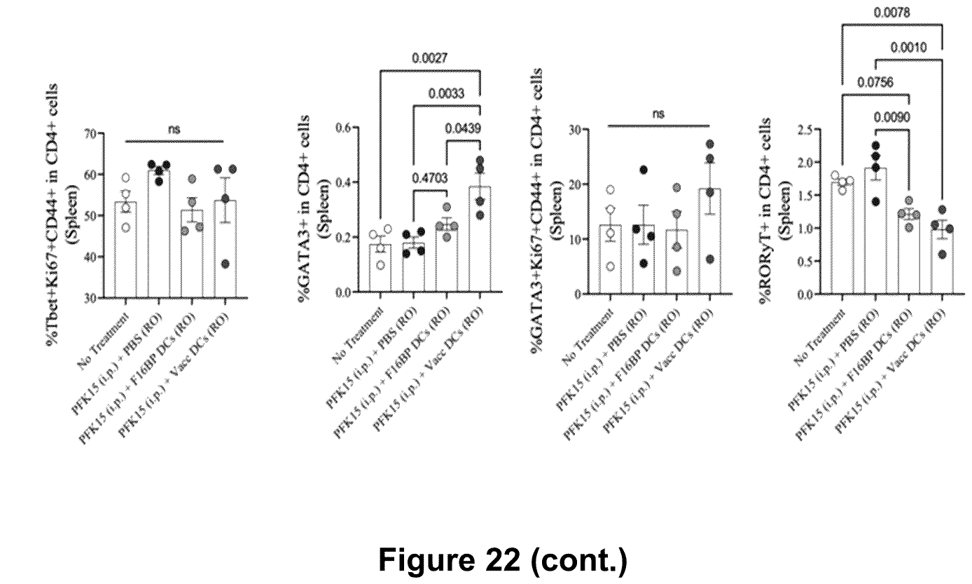

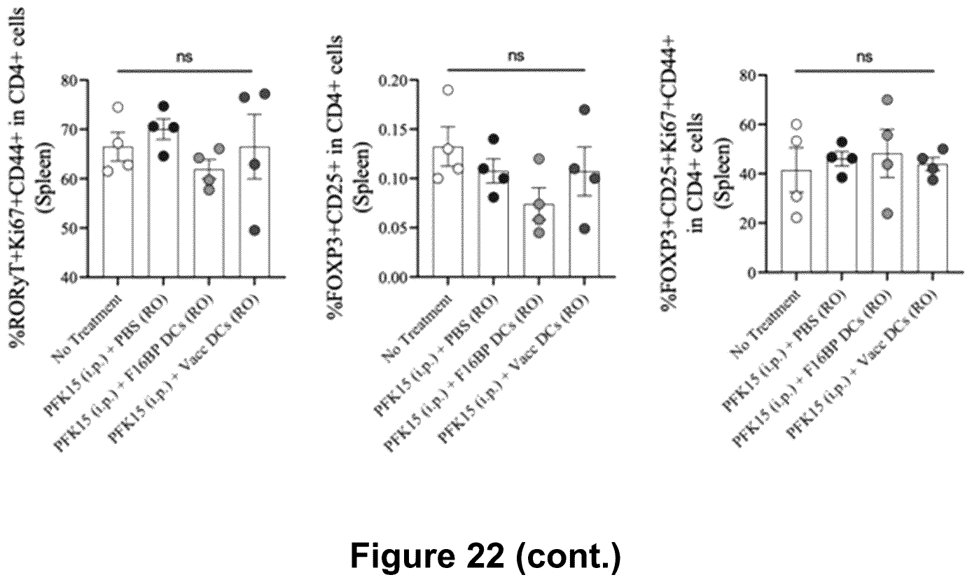

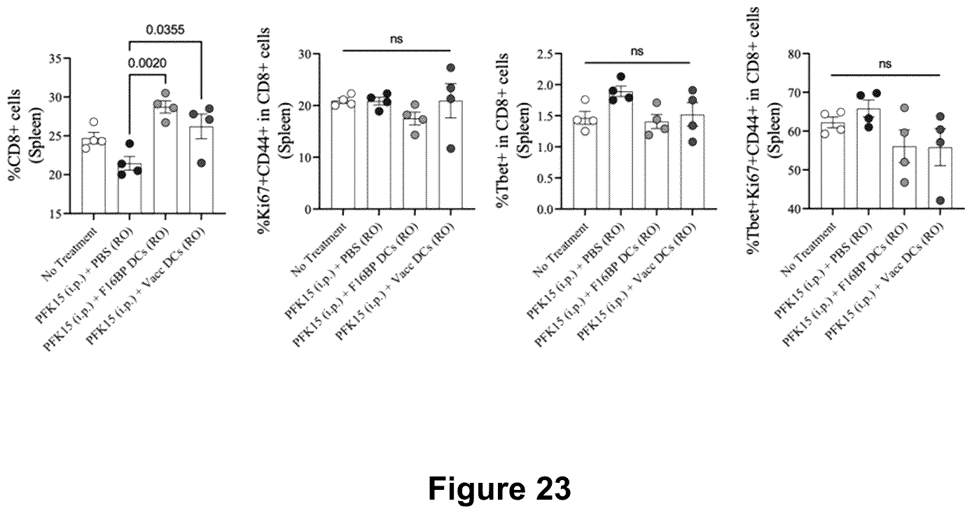

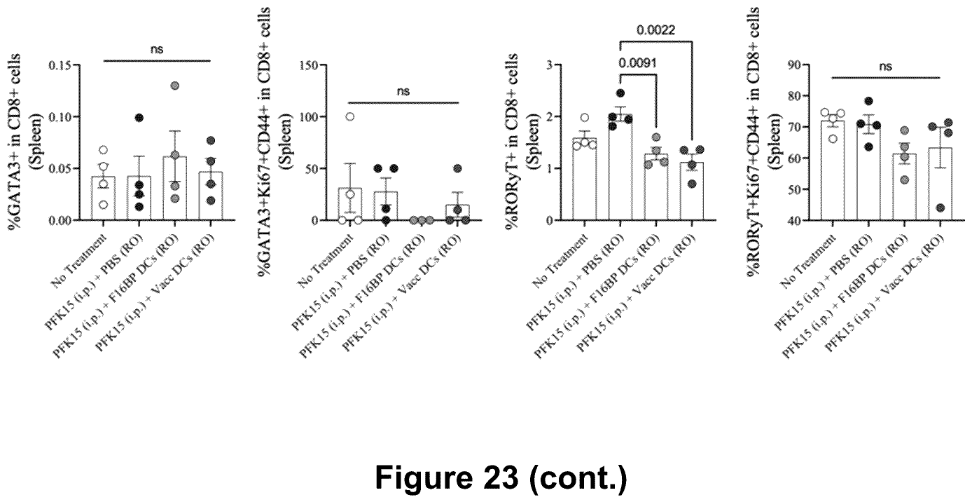

Since Vacc DCs are administered systemically, spleen and cervical lymph nodes were also analysed for the changes in innate and adaptive immune responses (FIG. 19 through FIG. 22). Notably, the overall frequency of DCs (CD11c+) and activated DCs (MHCII+CD86+ in CD11c+, MHCI+CD86+ in CD11c+, CD86+ in CD11c+, MHCI+ in CD11c+, MHCII+ in CD11c+) were significantly higher in spleen as compared to the controls; however, this difference was not observed in the cervical lymph nodes (FIG. 20 and FIG. 21). These findings suggest that the adoptive transfer of DCs primarily generate immune responses by modulating the innate immune responses in the spleen. Contrary to the tumor, it was observed that in the spleen, there was significant decreases in the Th17 frequency in Vacc DC groups as compared to all the controls (FIG. 22), significant increases in the Th2 frequency as compared to all the controls (FIG. 22), and significant decrease in Tc17 frequency as compared to PFK15 alone, and PFK15+F16BP DCs controls (FIG. 23). These findings indicated that although the Vacc DCs were administered systemically, the pro-inflammatory adaptive immune responses were primarily found in the tumor and not in the spleen.

This study demonstrated novel formulations that can be utilized to rescue glycolysis of DCs even in the presence of the glycolytic inhibitor. Importantly, this strategy generated a vaccine-based immunotherapy in the presence of glycolytic inhibitor chemotherapy. Importantly, it was observed that glycolytic rescue in DCs in two different melanoma model shows that cytotoxic T cells were increased in these treatment groups, potentially due to the rescue of DCs.

This work is important because it is versatile and can be utilized to different nodes in the metabolic pathways of cancer cells such as TCA cycle, glutaminolysis and mitochondrial respiration. It has been shown that the melanoma cells rely on glycolysis, which was targeted in this study. Moreover, it has been shown that PFKFB3 is an important target in the glycolytic pathway for retarding cancer cell growth. Therefore, clinical trials targeting different types of cancers have been carried out with varying success. This limited success can be because different activated immune cells, such as DCs, macrophages, and T cells, heavily rely on glycolysis for their energy needs, and for long-term cancer remission, it is important to have a robust immune response. This study demonstrated that DCs and other immune cells were rescued from glycolysis inhibition by either targeted delivery of metabolite-based formulation or via adoptive cell therapy.

In addition to the metabolite-based formulations, it has been extensively demonstrated that formulations can be developed that can be delivered subcutaneously for developing robust immunotherapy. However, these formulations are difficult to combine with chemotherapy, which are first line of defence in the clinic. Interestingly, in clinic in addition to glycolytic inhibitors other chemotherapy such as BRAF inhibitors are used, which also lead to decrease in glycolysis in DCs, and other immune cells. Therefore, a strategy that can improve glycolysis in immune cells will be highly beneficial.

In conclusion, this study demonstrated for the first time that the glycolysis of DCs can be rescued both in vitro and in vivo using a biomaterial strategy of delivering metabolites downstream of the inhibitory node. This strategy was tested using two different murine melanoma models in immunocompetent mice using two different treatment regimens to generate robust anti-melanoma innate and adaptive immune responses. Using these strategies tumor growth was significantly delayed, which was driven by Tc1 and Tc17 based responses within the tumor. These data demonstrated that metabolism-targeted chemotherapy can be combined with immunotherapies, by actively modulating DC metabolism. This strategy can be generalized to target other metabolic pathways that both cancer cells and immune cells utilize for their function.

Overall, inhibition of glycolysis in immune cells and cancer cells diminishes their activity, and thus combining immunotherapies with glycolytic inhibitors is challenging. Herein, a strategy was presented where glycolysis was inhibited in cancer cells using PFK15 (inhibitor of PFKFB3, rate-limiting step in glycolysis), while simultaneously glycolysis and function was rescued in DCs by delivery of fructose 1,6 biphosphate (F16BP, one-step downstream of PFKFB3). To demonstrate the feasibility of this strategy, vaccine formulations were generated using calcium phosphate chemistry, that incorporated F16BP, poly(IC) as adjuvant, and phosphorylated-TRP2 peptide antigen and tested in challenging and established YUMM1.1 tumors in immunocompetent mice. Furthermore, to test the versatility of this strategy, adoptive DC therapy was developed with formulations that incorporated F16BP, poly(IC) as adjuvant and mRNA derived from B16F10 cells as antigens in established B16F10 tumors in immunocompetent mice. F16BP vaccine formulations rescued DCs in vitro and in vivo, significantly improved the survival of mice, and generated cytotoxic T cell (Tc) responses by elevating Tc1 and Tc17 cells within the tumour. Overall, these results demonstrated that rescuing glycolysis of DCs using metabolite-based formulations generated immunotherapy even in the presence of glycolytic inhibitor.

Thus, described herein are systems and methods that demonstrate that glycolysis of DCs can be rescued both in vitro and in vivo using a biomaterial strategy of delivering metabolites downstream of the inhibitory node. For example, this strategy may comprise two different murine melanoma models in immunocompetent mice using two different treatment regimens to generate robust anti-melanoma innate and adaptive immune responses. This study demonstrates that metabolism-targeted chemotherapy can be combined with immunotherapies, by actively modulating DC metabolism and this strategy can be generalized to target other metabolic pathways that both cancer cells and immune cells utilize for their function.

1. A composition comprising a particle, wherein the particle comprises:
a) a nucleic acid molecule or a fragment or variant thereof comprising a nucleotide sequence that encodes at least one antigen or a fragment or variant thereof, and
b) a compound comprising at least one selected from the group consisting of:
i) a metabolite or a derivative or a polymer thereof; and
ii) the structure of Formula (I) 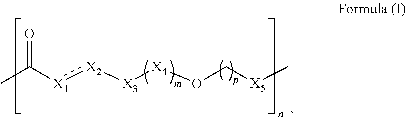
or a derivative or a pharmaceutical acceptable salt thereof,
wherein each occurrence of X₁ and X₂ is independently C═R₁, CR₂, or CR₃R₄;
each occurrence of X₃ and X₄ is independently C═R₁ or CR₃R₄;
each occurrence of Xₛ is independently O, S, C═R₁, CR₃R₄, NR₂, PR₂, or P(═R₁)(R₂);
the bond between X₁ and X₂ is a single bond or a double bond;
wherein when the bond between X₁ and X₂ is a single bond, X₁ and X₂ are each independently C═R₁ or CR₃R₄, and when the bond between X₁ and X₂ is a double bond X₁ and X₂ are each CR₂;
each occurrence of R₁ is independently selected from the group consisting of O, NH and S;
each occurrence of R₂, R₃, and R₄ is independently selected from the group consisting of hydrogen, hydroxyl, carboxyl, alkyl, cycloalkyl, heterocycloalkyl, aryl, and heteroaryl;
each occurrence of m is independently an integer represented by 0, 1, 2, or 5;
each occurrence of p is independently an integer from 1 to 50; and
each occurrence of n is independently an integer from 1 to 1000.

2. The composition of claim 1, wherein
a) the compound comprising the structure of Formula (I) is a compound comprising the structure of Formula (II) 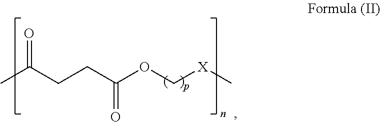
or a derivative or a pharmaceutical acceptable salt thereof,
wherein each occurrence of X is independently O, S, C═R₁, CR₃R₄, NR₂, PR₂, or P(═R₁)(R₂);
each occurrence of R₁ is independently selected from the group consisting of O, NH and S;
each occurrence of R₂, R₃, and R₄ is independently selected from the group consisting of hydrogen, hydroxyl, carboxyl, alkyl, cycloalkyl, heterocycloalkyl, aryl, and heteroaryl;
each occurrence of p is independently an integer from 1 to 15; and
each occurrence of n is independently an integer from 1 to 1000,
b) the metabolite comprises at least one selected from the group consisting of phosphoenolpyruvate, phosphoenolpyruvic acid, glucono-lactone-6-phosphate, gluconate-6-phosphase, sedoheptulose-7-phosphate, ribulose, ribulose-5-phosphate (R5P), xylulose, xylulose-5-phosphate, fructose-1,6-biphosphate (F16BP), fructose 6 phosphate (F6P), fructose-2,6-biphosphate, glycerate-2-phosphate, glucerate-3-phosphate, malate, fumarate, succinate, isocitrate, citrate, cis-aconitate, malonyl-CoA, acetyl CoA, 3-methylbutyryl CoA, 2-methylbutyryl CoA, 3-ketoacyl CoA, 3-hydroxyacyl CoA, enoyl CoA, 3-ketoacyl functionalized metabolite, 3-hydroxyacyl functionalized metabolite, enoyl functionalized metabolite, fatty acids, caprylic acid, capric acid, lauric acid, myristic acid, palmitic acid, stearic acid, arachidic acid, behenic acid, lignoceric acid, cerotic acid, myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid, docosahexaenoic acid, and any combination thereof;
c) the nucleic acid molecule comprises a nucleic acid molecule derived from a cancer cell, a synthetic nucleic acid molecule, an isolated mRNA or a fragment or variant thereof derived from a B16F10 cell, an isolated mRNA or a fragment or variant thereof derived from a YUMM cell, an isolated mRNA or a fragment or variant thereof derived from a 4T1 cell, or any combination thereof;
d) the antigen comprises a tumor antigen or a fragment or variant thereof; or
e) any combination of a), b), c), and d).

3. The composition of claim 1, further comprising an adjuvant.

4. The composition of claim 3, wherein the adjuvant
a) is operably linked to the metabolite or the derivative or the polymer thereof, the nucleic acid molecule, or both,
b) comprises at least one selected from the group consisting of polyinosinic:polycytidylic acid (poly(I:C)) or analog thereof, muramyl dipeptide derivatives (MDP) or analog thereof, Alum and Emulsions, complete Freund's adjuvant (CFA), incomplete Freund's adjuvant (IFA), pattern recognition receptor (PRR) ligands, cyclic guanosine monophosphate-adenosine monophosphate (2′3′-cGAMP), bis-(3′-5′)-cyclic dimeric adenosine monophosphate (c-di-AMP), Rp,Rp-isomer of the 2′3′-bisphosphorothioate analog of 3′3′-cyclic adenosine monophosphate (2′3′-c-di-AM(PS)2 (Rp,Rp)), cyclic diguanylate monophosphate-stimulator of interferon genes (c-di-GMP STING)-based vaccine adjuvant, CL401, CL413, CL429, Flagellin, Imiquimod, lipopolysaccharide (LPS) from the gram-negative bacteria E. coli 0111:B4 (LPS-EB), monophosphoryl lipid A from Salmonella minnesota R595 lipopolysaccharide (MPLA-SM), synthetic monophosphoryl lipid A (MPLA), oligodeoxynucleotides (ODN) 1585, ODN 1826, ODN 2006, ODN 2395, Pam3CSK4, Resiquimod (R848), trehalose-6,6-dibehenate (TDB), and any combination thereof; or
c) both a) and b).

5-7. (canceled)

8. The composition of claim 4, wherein the particle comprises a compound comprising the structure of 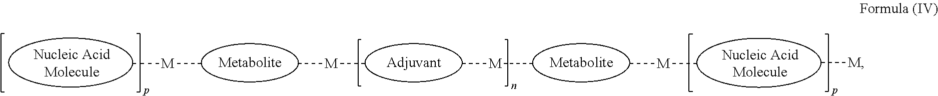
or a derivative or a pharmaceutical acceptable salt thereof, 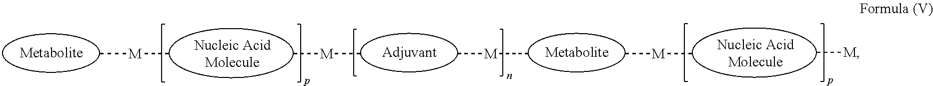
or a derivative or a pharmaceutical acceptable salt thereof, 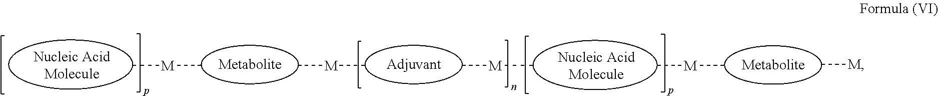
or a derivative or a pharmaceutical acceptable salt thereof, or 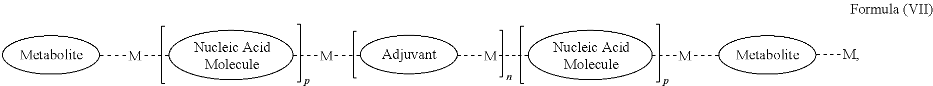
or a derivative or a pharmaceutical acceptable salt thereof,
wherein each occurrence of M is independently selected from the group consisting of: Ca, Mg, Na, K, Sr, Zn, Fe, Co, and Cu;
each occurrence of metabolite is independently a metabolite or derivative thereof,
each occurrence of n is independently an integer from 1 to 1000; and
each occurrence of p is independently an integer represented by 0 or 1.

9-15. (canceled)

16. The composition of claim 1, wherein the composition comprises calcium and phosphorous in a ratio of between about 2:about 1 to about 3:about 1.

17. The composition of claim 1, wherein the particle
a) induces glycolysis in at least one cell of interest,
b) has an average diameter of between about 1 μm to about 100 μm; or
any combination thereof.

18-20. (canceled)

21. The composition of claim 1, further comprising a therapeutic agent.

22. The composition of claim 21, wherein the therapeutic agent comprises at least one metabolic inhibitor, metabolic inhibitor comprising PFK15, glycolytic inhibitor, pentose phosphate pathway inhibitor, oxidative phosphorylation (OXPHOS) inhibitor, electron transport chain (ETC) inhibitor, polyamine pathway inhibitor, metabolic pathway accelerator, peptide therapeutic, protein therapeutic, enzyme to accelerate metabolism, or any combination thereof.

23-26. (canceled)

27. A method of inducing an immune response in a subject, wherein the method comprises administering to the subject a therapeutically effective amount of the composition of claim 1.

28. A method of inhibiting glycolysis in at least one cancer cell of interest and simultaneously inducing or rescuing glycolysis in at least one non-cancer cell of interest,
wherein the method comprises administering to the subject a therapeutically effective amount of the composition of claim 22.

29. A method of preventing or treating a disease or disorder, a metabolic inhibition of at least one cell, or a combination thereof in a subject in need thereof, wherein the method comprises administering a therapeutically effective amount of the composition comprising at least one particle,
wherein the particle comprises:
a) a compound comprising at least one selected from the group consisting of:
i) a metabolite or a derivative or a polymer thereof; and
ii) the structure of Formula (I) 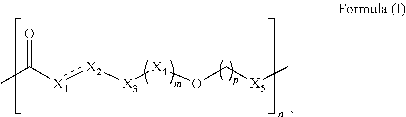
or a derivative or a pharmaceutical acceptable salt thereof,
wherein each occurrence of X₁ and X₂ is independently C═R₁, CR₂, or CR₃R₄;
each occurrence of X₃ and X₄ is independently C═R₁ or CR₃R₄;
each occurrence of X₅ is independently O, S, C═R₁, CR₃R₄, NR₂, PR₂, or P(═R₁)(R₂);
the bond between X₁ and X₂ is a single bond or a double bond;
wherein when the bond between X₁ and X₂ is a single bond, X₁ and X₂ are each independently C═R₁ or CR₃R₄, and when the bond between X₁ and X₂ is a double bond X₁ and X₂ are each CR₂;
each occurrence of R₁ is independently selected from the group consisting of O, NH and S;
each occurrence of R₂, R₃, and R₄ is independently selected from the group consisting of hydrogen, hydroxyl, carboxyl, alkyl, cycloalkyl, heterocycloalkyl, aryl, and heteroaryl;
each occurrence of m is independently an integer represented by 0, 1, 2, or 5;
each occurrence of p is independently an integer from 1 to 50; and
each occurrence of n is independently an integer from 1 to 1000; and
b) at least one selected from the group consisting of
i) a nucleic acid molecule or a fragment or variant thereof comprising a nucleotide sequence that encodes at least one antigen or a fragment or variant thereof, and
ii) an antigen or a fragment or variant thereof.

30. (canceled)

31. The method of claim 29, wherein the metabolite comprises at least one selected from the group consisting of phosphoenolpyruvate, phosphoenolpyruvic acid, glucono-lactone-6-phosphate, gluconate-6-phosphase, sedoheptulose-7-phosphate, ribulose, ribulose-5-phosphate (R5P), xylulose, xylulose-5-phosphate, fructose-1,6-biphosphate (F16BP), fructose 6 phosphate (F6P), fructose-2,6-biphosphate, glycerate-2-phosphate, glucerate-3-phosphate, malate, fumarate, succinate, isocitrate, citrate, cis-aconitate, malonyl-CoA, acetyl CoA, 3-methylbutyryl CoA, 2-methylbutyryl CoA, 3-ketoacyl CoA, 3-hydroxyacyl CoA, enoyl CoA, 3-ketoacyl functionalized metabolite, 3-hydroxyacyl functionalized metabolite, enoyl functionalized metabolite, fatty acids, caprylic acid, capric acid, lauric acid, myristic acid, palmitic acid, stearic acid, arachidic acid, behenic acid, lignoceric acid, cerotic acid, myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid, docosahexaenoic acid, and any combination thereof.

32. The method of claim 29, wherein the composition:
a) induces or rescues at least one selected from the group consisting of: a glycolysis, tricarboxylic acid (TCA) cycle, polyamine pathway, pentose phosphate pathway (PPP), activation of the at least one cell, extracellular acidification rate (ECAR), oxygen consumption rate (OCR), mitochondrial respiration, release of a metabolite, pro-inflammatory response, BRAF inhibitor, tyrosine kinase inhibitor, extracellular signal-regulated kinase (ERK inhibitor), MEK inhibitor, and cancer cell suppression,
b) induces or rescues glycolysis in at least one cell of interest and inhibits glycolysis in a cancer cell;
c) reduces or inhibits a tumor growth: reduces a cancer cell proliferation: or a combination thereof;
d) decreases the level of at least one immune suppressive cell:
e) increases the level of at least one selected from the group consisting of: a T cell, type 1 CD8+ T cell (Tc1), type 2 CD8+ T cell (Tc2), IL-17-producing CD8+ T cell (Tc17), T helper cell (Th), Th1, Th17, and effector T cell (Teff); or
f) any combination of a), b), c), d), and e).

33-36. (canceled)

37. The method of claim 29, wherein the disease or disorder is cancer.

38. The method of claim 29, wherein the method is an adoptive T cell therapy.

39. A method comprising administering the composition of claim 1 to a cell, a subject in need thereof, or both, wherein
a) he cell phagocytes the particle,
b) the subject has cancer; or
c) both a) and b).

40. The method of claim 39, wherein the cell is an immune cell.

41-42. (canceled)

43. The method of claim 39, wherein the method comprises the steps of:
a) administering the composition directly to the subject
b) administering the composition to the cell; and
administering the cell comprising the phagocyted particle to the subject; or
c) both a) and b).

44. (canceled)

45. The method of claim 39, wherein the method further comprises administering a glycolytic inhibitor to the subject.
